SEQUENCE LISTING (107 sequences)

SEQ ID NO: 1 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 2 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLDPEDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 3 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 4 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 5 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 6 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 7 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 8 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 9 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPGMLGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 10 - protein, 476 aa, Canis sp.
  source 1..476: note = Canis familiaris
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 11 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 12 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLDPEDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 13 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 14 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 15 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 16 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 17 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 18 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 19 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPGMLGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 20 - protein, 335 aa, Canis sp.
  source 1..335: note = Canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPENPEVQISWFVDGKQMQTAKTQPREEQFNGTYRVVSVLPIGHQDWLKGKQFTCKVNNKALPSSIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 21 - protein, 475 aa, Canis sp.
  source 1..475: note = Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 22 - protein, 471 aa, Canis sp.
  source 1..471: note = Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 23 - protein, 473 aa, Canis sp.
  source 1..473: note = Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSQSGSTVALACLVSGYIPEPVTVSWNSGSLTSGVHTFPSILQSSGLYSLSSMVTVPSSRWPSETFTCNVAHPATNTKVDKPVVKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 24 - protein, 471 aa, Canis sp.
  source 1..471: note = Canis familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASSTAPSVFPLAPSCGSTSGSTVALACLVSGYFPEPVTVSWNSGSLTSGVHTFPSVLKSSGLYSLSSMVTVPSSRLPSETFTCNVVHPATNTKVDKPVPKESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 25 - DNA, 894 bp, Homo sp.
  source 1..894: mol_type = genomic DNA; note = Homo sapiens
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 26 - DNA, 894 bp, Canis sp.
  source 1..894: mol_type = genomic DNA; note = Canis familiaris
atgacaacacccagaaattcaatgagtggaactctcccggtagatcctatgaaaagccctactgccatgtatcctgttcaaaaaataattcccaaaaggatgccttcagtggtgggccctacacaaaacttcttcatgagggaatctaagacactgggggctgtccagattatgaatgggctcttccacattgccctaggcagcctcctgatgattcacacggatgtctatgcgcccatctgtataactatgtggtaccctctctggggaggcattatgttcatcatttctggatcactcctggcagcagcggacaaaaaccccaggaagagtttggtcaaaggaaaaatgataatgaactcattgagcctctttgctgctatttctggaataatttttttgatcatggacatatttaatattaccatttcccatttttttaaaatggagaatttgaatcttattaaagctcccatgccatatgttgacatacacaactgtgacccagctaacccctctgagaaaaactctttatctatacaatattgtggcagcatacgatctgttttcttgggcgtttttgctgtgatggtgatctttacctttttccagaaacttgtgacagctggcattgttgagaatgaatggaaaaaactgtgctctaaacctaaatctgatgtagttgttctgttagctgctgaagaaaaaaaagaacagccgattgaaacaacagaagaaatggttgagctgactgaaatagcttcccaaccaaagaaagaagaagacattgaaattattccagtccaagaagaagaagaggaactggaaataaactttgcagaacctccccaggagcaggaatcttcaccaatagaaaacgacagcatcccttaa

SEQ ID NO: 27 - protein, 297 aa, Canis sp.
  source 1..297: note = Canis familiaris
MTTPRNSMSGTLPVDPMKSPTAMYPVQKIIPKRMPSVVGPTQNFFMRESKTLGAVQIMNGLFHIALGSLLMIHTDVYAPICITMWYPLWGGIMFIISGSLLAAADKNPRKSLVKGKMIMNSLSLFAAISGIIFLIMDIFNITISHFFKMENLNLIKAPMPYVDIHNCDPANPSEKNSLSIQYCGSIRSVFLGVFAVMVIFTFFQKLVTAGIVENEWKKLCSKPKSDVVVLLAAEEKKEQPIETTEEMVELTEIASQPKKEEDIEIIPVQEEEEELEINFAEPPQEQESSPIENDSIP

SEQ ID NO: 28 - DNA, 1895 bp, Felis sp.
  source 1..1895: mol_type = genomic DNA; note = Felis catus
tgcagccgcagaaacacaaggccctggggcggcagagagctatgccgggcggctggtctgcagacttcctggagggccaccccaagcccgggcacctgataccctgcccggaccctcccaggagcgaggccctggtggtcacacagcctcctctcttgcagcctccaccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccggccgtcctgcaggcctcggggctgtactctctcagcagcatggtgacagtgccctccagcaggtggctcagtgacaccttcacctgcaacgtggcccacccgcccagcaacaccaaggtggacaagaccggtaagagggtgtccactgggagacaggccaggtcagccggtccacctggaccccggggcacaggagaggcacctctgtcccccaagctcggggaggagggcctctgggcctggctgcctggtccagtgtgagcacaggctgcacagccccaccatacttcatctccactggcaggcaccagtctgggacgcccccctgacctgccctgacctagcccaccgcaagagctgtccccacccctggcccccactaacctccagtctggtctctctgcagtgcgcaaaacagaccacccaccgggacccaaaccctgcgactgtcccaaatgcccacgtgagtcaccagggcaccaccttgcacaccaaggcgatggcccgcgtccagggactgcctggtggggggaatgttcaagaggagcaccccccaaatgctgacccctgcattgtgtcttccacaccagcccctgagatgcttggaggaccgtccatcttcatcttccccccaaaacccaaggacaccctctcgatttcccggacgcccgaggtcacatgcttggtggtggacttgggcccagatgactccgatgtccagatcacatggtttgtggataacacccaggtgtacacagccaagacgagtccgcgtgaggagcagttcaacagcacctaccgtgtggtcagtgtcctccccatcctacaccaggactggctcaaggggaaggagttcaagtgcaaggtcaacagcaaatccctcccctcccccatcgagaggaccatctccaaggccaaaggtgggcaacaggacagatggggcacagggaggtcgagtggggcctggcggacccaggccagccctccactctgggagtgaccatctgtgctgacctctgaccccacaggacagccccacgagccccaggtgtacgtcctgcctccagcccaggaggagctcagcaggaacaaagtcagtgtgacctgcctgatcaaaagcttccacccgcctgacattgccgtcgagtgggagatcaccggacagccggagccagagaacaactaccggacgaccccgccccagctggacagcgacgggacctacttcgtgtacagcaagctctcggtggacaggtcccactggcagaggggaaacacctacacctgctcggtgtcacacgaagctctgcacagccaccacacacagaaatccctcacccagtctccgggtaaatgagcagcgcgcccagccccccaggaggcccccgcgggctctgagcgcccacccctgtgtacatcccccaccccgggcaggtaccctgcgtgaaataaagcacccagcactgccctgggacctaggacactgtcgtgcgtctttccaaggcagagctcaggacacccgggcctctgggaggtgcgggcaggccaggctccgaggctcaggggtcaccat

SEQ ID NO: 29 - DNA, 1895 bp, Felis sp.
  source 1..1895: mol_type = genomic DNA; note = Felis catus
tgcagccgcagaaacacgaggccctggggcggcagagagctacgccgggcggctggtctgcagacttcctggagggccaccccaagcccgggcacctgataacctgcccggaccctcccaggagcgaggccctggtggtcacacagcctcctctcttgcagcctcccccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccatccgtcctgcaggcctcggggctctactctctcagcagcatggtgacagtgccctccagcaggtggctcagtgacaccttcacctgcaacgtggcccaccggcccagcagcaccaaagtggacaagaccggtaagagggtgtccactgggagacaggccaggtcagccggtccacctggaccccggggcacaggagaggcacctctgtcccccaagctcggggaggagggcctctgggcctggctgcctggtccagtgtgagcacaggctgcacagccccaccatacttcatctccactggcaggcaccagtctgggacgcccccctgacctgccctgacctagcccaccccaagagctgtccccacccctggcccccactaacctccagtctggtctctctgcagtacccaaaacagcgtctacaatagagtccaaaaccggggaaggtcccaaatgcccaggtgagtcaccagggcaccaccttgcacaccaaggcaatggcccgcatccagggactgcctggtggggggaatgttcaagaggatcccccccaaatgctgacccctgcattgtgtctcccacaccagttcctgagattcctggagcaccgtccgtcttcatcttccccccaaaacccaaggacaccctctcgatttcccggacgcccgaggtcacgtgcttggtggtggacttgggcccagatgactccaatgtccagatcacatggtttgtggataacaccgagatgcacacagccaagacgaggccgcgtgaggagcagttcaacagcacctaccgtgtggtcagtgtcctccccatcctacaccaggactggctcaaggggaaggagttcaagtgcaaggtcaacagcaaatccctcccctctgccatggagaggaccatctccaaggccaaaggtgggcaacaggacagatggggcacagggaggtcgagtggggcctggtggacccaggccagccctccactctgggagtgaccatctgtgctgacctctgaccccacaggacagccccacgagccccaggtgtacgtcctgcctccaacccaggaggagctcagcgagaacaaagtcagtgtgacctgcctgatcaaaggcttccacccgcctgacattgccgtcgagtgggagatcaccggacagccggagccagagaacaactaccagacgaccccgccccagctggacagcgacgggacctacttcctgtacagcaggctctcggtggacaggtcccactggcagaggggaaacacctacacctgctcggtgtcacacgaagctctgcacagccaccacacacagaaatccctcacccaatctccgggtaaatgagcagcgcgcccagccccccaggaggcccccgcgggctctgagcgcccacccctgtgtacatcccccccccccgggcaggcaccctgcatgaaataaagcacccagcactgccctgggacctgtgacactgtcgtgcgtctttccaaggcagagctcaggacacccgggcctctgggaggtgcgggcaggccaggctctgaggctcaggggtcaccat

SEQ ID NO: 30 - DNA, 1869 bp, Felis sp.
  source 1..1869: mol_type = genomic DNA; note = Felis catus
cagccgcagaaacatgaggtcctggggcggcagagagctacgccgggcggctggtctgcagacttcctggagggccaccccaagcccgggcacctgataccctgcccggaccctcccaggagcgaggccctggtggtcacacagcctcctctcttgcagcctcccccaaggccccatcggtgttcccacgggcccccagctgtgggaccatatctggcgccaccgtggccctgatctgcctggtgttagactacttccctgagccggtgaccgtgtcctggaagcccggcgccctgaccaacggtgtgcacaccttcccggccgtcctgcagccctcggggctctactctctcagcagcatggtgacagtgcccaccagcagctggctcagagacaccttcacctgcaacgtggcccacccgcccagcaacaccaaggtggaaaagatcggtaagagggtgtccactgggagacaggccaggtcagccggtccacctggaccccggggcacaggagaggcacctctgtcccccaagctcggggaggagggcctctgggcctggctgcctggtccagtgtgagcacaggctgcacagccccaccatacttcatctccactggcaggcaccagtctgggacgcccccctgacctgccctgacctagcccaccccaagagctgtccccacccctggcccccactaacctccagtctggtctctctgcagtgctcccaccctgcaagtgtcccaaatgcccaggtgagtcagcagggcaccgccctgcacaccaaggccatggtccgtatccagggactgcctggtggggggaatgttcaagaggagcaccccccaaatgctgacccccgcattgtgtctcccacaccagtacctgagattcccgggggaccgtccgtcttcatcttccccccaaaacccaaggacaccctctcgatttcccggacgcccgaggtcacgtgcttggtggtggacttgggcccagatgactccaatgtccagatcacatggtttgtggataacaccgagatgcacacagccaagacgagaccgcgtgaggagcagttcaacagcacctaccgtgtggtcagtgtcctccccatcgtgcaccaggactggctcacggggaaggagttcaagtgcaaggtcaacagcaaagccctcccctctgccatcgagaggaccatctccaaggccaaaggtgggcaacaggacagatggggcacagggaggtcgagtggggcctggcggacccaggccagccctccactctgggagtgaccatctgtgctgacctctgaccccacaggacagccccacgagccccaggtgtacgtcctgcctccagcccaggaggagctcagcgagaacaaagtctgtgtgacctgcctgatcaaaggcttctacccgcctgacattgccgtcgagtgggagatcaccggacagccggagccagagaacaactaccggacgaccccgccccagctggacagcgacgggacctacttcgtgtacagcaggctctcgatggacaggtcccgctggcagagtggaaacacctacacctgctcggtgtcacacgaagctctgcacagccaccacacacagaaatccctcacccagtctccgggtaaatgagcagcgcgcccagccccccagcaggcccccgcgggctctgagcgcccacccctgtgtacatcccccaccccgggcaggtaccctgcgtgaaataaagcacccagcactgccctgggacctgtgacactgtcgtgtgtctttccaaggcagagctcaggacacccgggcctctgggaggtgcgggcaggccaggctccaaggctcaggggtcaccat

SEQ ID NO: 31 - protein, 334 aa, Felis sp.
  source 1..334: note = Felis catus
STTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 32 - protein, 334 aa, Felis sp.
  source 1..334: note = Felis catus
SPTAPSVFPLAPSCGTTSGATVALACLVLGYFPEPVTVSWNSGALTSGVHTFPSVLQASGLYSLSSMVTVPSSRWLSDTFTCNVAHRPSSTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 33 - protein, 326 aa, Felis sp.
  source 1..326: note = Felis catus
SPKAPSVFPRAPSCGTISGATVALICLVLDYFPEPVTVSWKPGALTNGVHTFPAVLQPSGLYSLSSMVTVPTSSWLRDTFTCNVAHPPSNTKVEKIVLPPCKCPKCPVPEIPGGPSVFIFPPKPKDTLSISRTPEVTCLVVDLGPDDSNVQITWFVDNTEMHTAKTRPREEQFNSTYRVVSVLPIVHQDWLTGKEFKCKVNSKALPSAIERTISKAKGQPHEPQVYVLPPAQEELSENKVCVTCLIKGFYPPDIAVEWEITGQPEPENNYRTTPPQLDSDGTYFVYSRLSMDRSRWQSGNTYTCSVSHEALHSHHTQKSLTQSPGK

SEQ ID NO: 34 - protein, 476 aa, Felis sp.
  source 1..476: note = Felis catus
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSSIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 35 - protein, 476 aa, Felis sp.
  source 1..476: note = Felis catus
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLGSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 36 - protein, 476 aa, Felis sp.
  source 1..476: note = Felis catus
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEIPGAPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 37 - protein, 476 aa, Felis sp.
  source 1..476: note = Felis catus
MELGLSWIFLLAILKGVQCEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEIPGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSAIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 38 - protein, 335 aa, Felis sp.
  source 1..335: note = Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSSIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 39 - protein, 335 aa, Felis sp.
  source 1..335: note = Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEAAGGPSIFIFPPKPKDTLSISRTPEVTCLVVDLGPDDSDVQITWFVDNTQVYTAKTSPREEQFNSTYRVVSVLPILHQDWLKGKEFKCKVNSKSLGSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 40 - protein, 335 aa, Felis sp.
  source 1..335: note = Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEIPGAPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 41 - protein, 335 aa, Felis sp.
  source 1..335: note = Felis catus
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEIPGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSAIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 42 - DNA, 20 bp, synthetic construct
  source 1..20: mol_type = other DNA
tgcagccgcagaaacacaag

SEQ ID NO: 43 - DNA, 20 bp, synthetic construct
  source 1..20: mol_type = other DNA
atggtgacccctgagcctcg

SEQ ID NO: 44 - DNA, 20 bp, synthetic construct
  source 1..20: mol_type = other DNA
tgcagccgcagaaacacgag

SEQ ID NO: 45 - DNA, 20 bp, synthetic construct
  source 1..20: mol_type = other DNA
atggtgacccctgagcctca

SEQ ID NO: 46 - DNA, 21 bp, synthetic construct
  source 1..21: mol_type = other DNA
cagccgcagaaacatgaggtc

SEQ ID NO: 47 - DNA, 20 bp, synthetic construct
  source 1..20: mol_type = other DNA
atggtgacccctgagccttg

SEQ ID NO: 48 - protein, 7 aa, Canis sp.
  source 1..7: note = Canis familiaris
EPLGGPS

SEQ ID NO: 49 - protein, 7 aa, Canis sp.
  source 1..7: note = Canis familiaris
EMLGGPS

SEQ ID NO: 50 - protein, 7 aa, Canis sp.
  source 1..7: note = Canis familiaris
GLLGGPS

SEQ ID NO: 51 - protein, 7 aa, Canis sp.
  source 1..7: note = Canis familiaris
ESLGGPS

SEQ ID NO: 52 - protein, 7 aa, Canis sp.
  source 1..7: note = Canis familiaris
HIDLPSP

SEQ ID NO: 53 - protein, 7 aa, Canis sp.
  source 1..7: note = Canis familiaris
NKALPSP

SEQ ID NO: 54 - protein, 7 aa, Canis sp.
  source 1..7: note = Canis familiaris
NKALPSP

SEQ ID NO: 55 - protein, 7 aa, Canis sp.
  source 1..7: note = Canis familiaris
HIGLPSP

SEQ ID NO: 56 - protein, 6 aa, Canis sp.
  source 1..6: note = Canis familiaris
DLGRED

SEQ ID NO: 57 - protein, 6 aa, Canis sp.
  source 1..6: note = Canis familiaris
DLDPED

SEQ ID NO: 58 - protein, 6 aa, Canis sp.
  source 1..6: note = Canis familiaris
DLDPEN

SEQ ID NO: 59 - protein, 6 aa, Canis sp.
  source 1..6: note = Canis familiaris
DLGRED

SEQ ID NO: 60 - protein, 7 aa, Canis sp.
  source 1..7: note = Canis familiaris
ESLGGPS

SEQ ID NO: 61 - protein, 7 aa, Canis sp.
  source 1..7: note = Canis familiaris
ESLGGPS

SEQ ID NO: 62 - protein, 7 aa, Canis sp.
  source 1..7: note = Canis familiaris
ESLGGPS

SEQ ID NO: 63 - protein, 7 aa, Canis sp.
  source 1..7: note = Canis familiaris
ESLGGPS

SEQ ID NO: 64 - protein, 7 aa, Canis sp.
  source 1..7: note = Canis familiaris
EAAGGPS

SEQ ID NO: 65 - protein, 7 aa, Canis sp.
  source 1..7: note = Canis familiaris
EAAGGPS

SEQ ID NO: 66 - protein, 7 aa, Canis sp.
  source 1..7: note = Canis familiaris
EAAGGPS

SEQ ID NO: 67 - protein, 7 aa, Canis sp.
  source 1..7: note = Canis familiaris
GMLGGPS

SEQ ID NO: 68 - protein, 7 aa, Canis sp.
  source 1..7: note = Canis familiaris
EAAGGPS

SEQ ID NO: 69 - protein, 7 aa, Canis sp.
  source 1..7: note = Canis familiaris
HIGLPSP

SEQ ID NO: 70 - protein, 7 aa, Canis sp.
  source 1..7: note = Canis familiaris
HIGLPSP

SEQ ID NO: 71 - protein, 7 aa, Canis sp.
  source 1..7: note = Canis familiaris
HIGLPSS

SEQ ID NO: 72 - protein, 7 aa, Canis sp.
  source 1..7: note = Canis familiaris
HIGLGSP

SEQ ID NO: 73 - protein, 7 aa, Canis sp.
  source 1..7: note = Canis familiaris
NKALPSP

SEQ ID NO: 74 - protein, 7 aa, Canis sp.
  source 1..7: note = Canis familiaris
NKALPSS

SEQ ID NO: 75 - protein, 7 aa, Canis sp.
  source 1..7: note = Canis familiaris
NKALGSP

SEQ ID NO: 76 - protein, 7 aa, Canis sp.
  source 1..7: note = Canis familiaris
NKALPSP

SEQ ID NO: 77 - protein, 7 aa, Canis sp.
  source 1..7: note = Canis familiaris
NKALPSS

SEQ ID NO: 78 - protein, 6 aa, Canis sp.
  source 1..6: note = Canis familiaris
DLDPED

SEQ ID NO: 79 - protein, 6 aa, Canis sp.
  source 1..6: note = Canis familiaris
DLGRED

SEQ ID NO: 80 - protein, 6 aa, Canis sp.
  source 1..6: note = Canis familiaris
DLGRED

SEQ ID NO: 81 - protein, 6 aa, Felis sp.
  source 1..6: note = Felis catus
DLGRED

SEQ ID NO: 82 - protein, 6 aa, Felis sp.
  source 1..6: note = Felis catus
DLDPED

SEQ ID NO: 83 - protein, 6 aa, Felis sp.
  source 1..6: note = Felis catus
DLDPED

SEQ ID NO: 84 - protein, 6 aa, Felis sp.
  source 1..6: note = Felis catus
DLDPED

SEQ ID NO: 85 - protein, 6 aa, Felis sp.
  source 1..6: note = Felis catus
DLDPEN

SEQ ID NO: 86 - protein, 6 aa, Felis sp.
  source 1..6: note = Felis catus
DLDPEN

SEQ ID NO: 87 - protein, 7 aa, Felis sp.
  source 1..7: note = Felis catus
EMLGGPS

SEQ ID NO: 88 - protein, 7 aa, Felis sp.
  source 1..7: note = Felis catus
EIPGAPS

SEQ ID NO: 89 - protein, 7 aa, Felis sp.
  source 1..7: note = Felis catus
EIPGGPS

SEQ ID NO: 90 - protein, 7 aa, Felis sp.
  source 1..7: note = Felis catus
SKSLPSP

SEQ ID NO: 91 - protein, 7 aa, Felis sp.
  source 1..7: note = Felis catus
SKSLPSA

SEQ ID NO: 92 - protein, 7 aa, Felis sp.
  source 1..7: note = Felis catus
SKALPSA

SEQ ID NO: 93 - protein, 6 aa, Felis sp.
  source 1..6: note = Felis catus
DLGPDD

SEQ ID NO: 94 - protein, 6 aa, Felis sp.
  source 1..6: note = Felis catus
DLGPDD

SEQ ID NO: 95 - protein, 6 aa, Felis sp.
  source 1..6: note = Felis catus
DLGPDD

SEQ ID NO: 96 - protein, 7 aa, Felis sp.
  source 1..7: note = Felis catus
EAAGGPS

SEQ ID NO: 97 - protein, 7 aa, Felis sp.
  source 1..7: note = Felis catus
EAAGGPS

SEQ ID NO: 98 - protein, 7 aa, Felis sp.
  source 1..7: note = Felis catus
EIPGAPS

SEQ ID NO: 99 - protein, 7 aa, Felis sp.
  source 1..7: note = Felis catus
EIPGGPS

SEQ ID NO: 100 - protein, 7 aa, Felis sp.
  source 1..7: note = Felis catus
SKSLPSS

SEQ ID NO: 101 - protein, 7 aa, Felis sp.
  source 1..7: note = Felis catus
SKSLGSP

SEQ ID NO: 102 - protein, 7 aa, Felis sp.
  source 1..7: note = Felis catus
SKSLPSP

SEQ ID NO: 103 - protein, 7 aa, Felis sp.
  source 1..7: note = Felis catus
SKSLPSA

SEQ ID NO: 104 - protein, 6 aa, Felis sp.
  source 1..6: note = Felis catus
DLGPDD

SEQ ID NO: 105 - protein, 6 aa, Felis sp.
  source 1..6: note = Felis catus
DLGPDD

SEQ ID NO: 106 - protein, 6 aa, Felis sp.
  source 1..6: note = Felis catus
DLGPDD

SEQ ID NO: 107 - protein, 6 aa, Felis sp.
  source 1..6: note = Felis catus
DLGPDD

(19) United States
(12) Patent Application Publication
(10) Pub. No.: US 2025/0340663 A1
(43) Pub. Date: Nov. 6, 2025

(54) ANTIBODY FC VARIANTS

(71) Applicant: PETMEDIX LTD., Cambridge (GB)

(72) Inventors: Meng Amy LI, Cambridge (GB); Roberto BANDIERA, Cambridge (GB); Marco BARDELLI, Cambridge (GB); Yasmin Zoe PATERSON, Cambridge (GB); Andreas MARQUARDT, Cambridge (GB)

(21) Appl. No.: 18/681,593

(22) PCT Filed: Aug. 5, 2022
(86) PCT No.: PCT/GB2022/052061

§ 371 Date: Feb. 6, 2024

(30) Foreign Application Priority Data
Aug. 6, 2021 (GB) 2111363.4

(51) Int. Cl. C07K 16/28; A61K 39/00

(52) U.S. Cl. CPC C07K 16/2887; A61K 2039/505; C07K 2317/24; C07K 2317/52; C07K 2317/732; C07K 2317/734; C07K 2317/92; C07K 2317/94

(57) ABSTRACT

The invention relates to variant Fc polypeptides of companion animals having reduced effector functions, including reduced antibody-dependent cellular cytotoxicity (ADCC) and complement mediated cytotoxicity (CDC). Effector function null IgG is important for a number of antibody mechanisms of action in a wide range of disease areas and in Fc-fusion proteins and alternative antibody formats.

### INTRODUCTION

Antibody-based therapeutics have emerged as important components of therapies for an increasing number of human malignancies in such fields as oncology, inflammatory and infectious diseases. Indeed, antibodies are one of the best-selling classes of drugs today; five of the top ten best selling drugs are antibodies. Increasingly, antibody therapy is also used in veterinary medicine for the treatment of domestic animals, such as dogs.

There is a huge need for these therapies in veterinary medicine, as in the USA alone there are 6M cases of cancer diagnosed each year in dogs, and a similar number in cats (Cekanova and Rathore Animal models and therapeutic molecular targets of cancer: utility and limitations. Drug Des Devel Ther, 8:1911-2, 2014) “Animal models and therapeutic molecular targets of cancer: utility and limitations” Drug design, development and therapy 8:1911-1922). Moreover, one in four American dogs is diagnosed with some form of arthritis (Bland “Canine osteoarthritis and treatments: a review” Veterinary Science Development 5(2)), 2015)). Thus, there is potential for application of antibody therapeutics to many chronic veterinary diseases. Monoclonal antibodies could also be beneficial for the detection, prevention and control of parasitic, bacterial and viral diseases.

The first monoclonal antibody for therapeutic use in humans received marketing approval 25 years ago, 80 have since been approved and more than 50 are in late-stage clinical development. In contrast, the use of antibodies in veterinary medicine is in its early stages with just a few antibodies under development. The limited progress reflects the fact that developing species-specific therapeutic antibodies is technically challenging and only a relatively recent endeavour. There is therefore a need to develop improved antibodies for veterinary medicine, as well as methods for making antibodies for veterinary medicine.

Antibody structure has been exploited to engineer a variety of different human antibody formats to target disease in humans. An example is optimization of the human Fc domain. Underlying the optimization of the Fc is modulating its ability to bind to Fc receptors, C1q, and FcRn. The Fc domain can be modified to obtain beneficial gain-of-function modifications, but in some cases, it can be beneficial to abolish antibody Fc function. These situations include antibodies that are used as receptor agonists to crosslink receptors and induce signaling, (receptor antagonists to block receptor:ligand interactions to prevent signaling, or drug delivery vehicles to deliver drugs or a drug to antigen-expressing target cells. In these instances, Fc engagement of receptors on effector cells or engagement of C1q is not wanted, because it can lead to undesired killing of biologically-important cells expressing the receptor or recruitment of drug-conjugated antibodies to off-target cells. Effector function null IgG is thus important for a number of antibody mechanisms of action in a wide range of disease areas. In addition, this is also important in Fc-fusion proteins and alternative antibody formats such as bi- or multi-specific antibodies. Several strategies to manipulate FcγR binding of human antibodies and complement protein C1q binding, including changes to Fc sequence and glycosylation, are described in Saunders Front. Immunol., Article 1296, Volume 10, 7 Jun. 2019.

However, changes in Fc-mediated functions by editing amino acid sequences in the Fc region may result in changes in biophysical properties, which may lead to undesired outcomes such as reduced thermostability, increased aggregation propensity and compromised in vivo pharmacokinetics, for further development of Fc-based therapeutics. In addition, modifications can also cause conformational changes which may impact Fc interactions with other molecules such as protein A and neonatal receptor (FcRn) binding. These changes are unpredictable based on sequence alone. Therefore, when modifying Fc fragments to alter effector functions, it is important to consider and carefully evaluate the impact of any mutations on physicochemical properties, including stability and aggregation and also binding to protein A and FcRn. This is critical in developing clinically useful molecules.

There is a need to develop improved antibodies for veterinary medicine that have reduced or abolished Fc binding, as well as methods for making such antibodies for veterinary medicine. The invention is aimed at addressing this need, in particular by providing modified immunoglobulin Fc regions.

### SUMMARY OF THE INVENTION

The ability to mediate cytotoxic and phagocytic effector functions are potent mechanisms by which antibodies destroy targeted cells. The Fc region links the recognition domain of antibodies to these effector functions through an interaction with Fc receptors and ligands. Manipulation of these effector functions by alteration of the Fc region has important implications in the treatment of numerous medical conditions, for example cancer, autoimmune diseases and infectious diseases.

The invention provides modified canine and modified feline Fc regions (compared to wt) with advantageous properties.

It is known that canine IgG-A and IgG-D are effector function deficient, while IgG-B and IgG-C are effector function proficient. However, IgG-B is the predominant IgG currently being used in therapeutic antibodies due to its other characteristics such as protein A binding capacity and relatively long half life compared to other canine IgGs. Therefore, generating effector function null canine IgG-B is attractive for certain therapeutic areas.

The inventors have modified canine IgG-B to reduce or abolish canine IgG-B effector function when compared to the same polypeptide comprising a wild-type IgG-B Fc domain. Based on structural analysis, the regions targeted in the amino acid sequence of the Fc domain for modification included the lower hinge, proline region and SHED region, based on the amino acid sequence motifs, where potential interactions with FcgammaR and C1q occur. The effect of the Fc variants in fully canine cell-based functional assays was demonstrated.

Although less is known about feline IgGs, previous work on characterisation of natural feline IgG isoforms has determined two isoforms, termed IgG1 and IgG2. IgG1 has been shown to be effector function proficient, while IgG2 is effector function deficient. However, IgG2 exerts hinge disulphide bond instability, akin to human IgG4. This hinders the application of wild type IgG2 to be directly used in therapeutic applications. Therefore, modification of feline IgG1 to abolish its effector function is an attractive option to obtain stable effector function null feline IgG variants for certain therapeutic areas.

Using the same approaches to canine IgG-B modifications, the inventors have modified feline IgG1 to reduce or abolish feline IgG1 effector function when compared to the same polypeptide comprising a wild-type feline IgG1 Fc domain. Based on structural analysis, the regions targeted in the amino acid sequence of the Fc domain for modification included the lower hinge, proline region and SHED region, based on the amino acid sequence motifs, where potential interactions with FcγR and C1q occur. The effect of the Fc variants was demonstrated in cell-based functional assays.

The present invention thus provides isolated companion animal, for example canine, feline and equine, Fc variants, including speciated variants, that provide reduced complement- and FcγR-mediated effector functions. The Fc variants are part of isolated polypeptides, e.g. antibodies or antibody fragments.

The Fc variants of the invention comprise one or more amino acid substitutions relative to wild type Fc, wherein said substitution(s) alter binding to complement protein C1q or reduce CDC, ADCC and/or ADCP activity. Thus, the invention relates to Fc variants, for example canine, equine or feline variants, with one or more modifications in the Fc domain compared to the native wild type sequence wherein the modification is an amino acid substitution in the lower hinge, proline region and SHED region.

With reference to wild type canine IgG-B constant region sequence as shown in SEQ ID NO. 11 and feline IgG1 constant region sequence as shown in SEQ ID NO. 31, the lower hinge comprises residues 119-125, the proline region comprises residues 211-217 and the SHED region comprises residues 151-156. FIG. 2A and FIG. 15A illustrates the domains and the residue numbering for canine and feline constant regions respectively. Canine variant sequences according to the invention in the lower hinge, proline region and SHED region compared to wild type are shown in FIG. 2A and the feline variant sequences according to the invention in the lower hinge, proline region and SHED region compared to wild type shown in FIG. 15A. The numbering of residues is with reference to SEQ ID NO: 11 (FIG. 2B) for canine Fc and with reference to SEQ ID NO:31 (FIG. 15B) for feline Fc. These reference sequences show the canine IgGB and feline IgG1 wild type constant region amino acid sequence respectively. FIG. 1 shows full canine IgG isoform wild type sequences and FIG. 13 shows previously identified feline IgG1 and IgG2 isoforms. It also shows a novel isoform, IgG3, which was identified, isolated and characterised by the inventors.

Thus, the invention relates to a canine, chimeric or caninised IgG-B polypeptide or Fc region thereof as further described below. The invention relates to a feline, chimeric or felinised IgG1 polypeptide or Fc region thereof as further described below. The invention relates to an isolated feline IgG3 polypeptide or portion thereof and recombinant antibody molecules comprising an IgG3 polypeptide or portion thereof.

In one aspect, the invention relates to a canine, chimeric or caninised IgG-B polypeptide or Fc region thereof comprising a modified Fc region wherein said Fc region comprises
a) an amino acid substitution selected from a modification at position 119 of SEQ ID NO: 11 to G, an amino acid substitution at position 120 of SEQ ID NO: 11 to S or A and/or an amino acid substitution at position 121 of SEQ ID NO: 11 to A; and/or
b) an amino acid substitution at position 156 of SEQ ID NO: 11 to N and/or
c) an amino acid substitution is selected from one of the following modifications
c1) a modification at position 211 of SEQ ID NO: 11 to H in combination with an amino acid substitution at position 212 of SEQ ID NO: 11 to I and an amino acid substitution at position 213 of SEQ ID NO: 11 to G;
c2) an amino acid substitution at position 215 of SEQ ID NO: 11 to G;
c3) an amino acid substitution at position 217 of SEQ ID NO: 11 to S or
c4) a combination of c1 to c3.

In one aspect, the invention relates to a canine, chimeric or caninised IgG-B polypeptide or Fc region thereof wherein said Fc region comprises an amino acid substitution as set out in a) below and optionally an amino acid substitution as set out in b) below and/or c) below wherein
a) the amino acid substitution is selected from a modification at position 119 of SEQ ID NO: 11 to G, an amino acid substitution at position 120 of SEQ ID NO: 11 to S or A and/or an amino acid substitution at position 121 of SEQ ID NO: 11 to A;
b) the amino acid substitution is selected from a modification at position 153 of SEQ ID NO: 11 to G, an amino acid substitution at position 154 of SEQ ID NO: 11 to R and/or an amino acid substitution at position 156 of SEQ ID NO: 11 to N and/or
c) the amino acid substitution is selected from a modification at position 211 of SEQ ID NO: 11 to H, an amino acid substitution at position 212 of SEQ ID NO: 11 to I, an amino acid substitution at position 213 of SEQ ID NO: 11 to G, an amino acid substitution at position 215 of SEQ ID NO: 11 to G and/or an amino acid substitution at position 217 of SEQ ID NO: 11 to S.

In one aspect, the invention relates to a canine, chimeric or caninised IgG-B polypeptide or Fc region thereof wherein said Fc region comprises
1) an Fc region comprising
a) an amino acid substitution at position 120 of SEQ ID NO: 11 to S and
b) an amino acid substitution at position 211 of SEQ ID NO: 11 to H, an amino acid substitution at position 212 of SEQ ID NO: 11 to I and an amino acid substitution at position 213 of SEQ ID NO: 11 to G or
2) an Fc region comprising
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G or
3) an Fc region comprising
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 217 of SEQ ID NO: 11 to S or
4) an Fc region comprising
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 215 of SEQ ID NO: 11 to G or
5) an Fc region comprising amino acid substitution at position 120 of SEQ ID NO: 11 to A and at position 121 to A or
6) an Fc region comprising
a) an amino acid substitution at position 120 of SEQ ID NO: 11 to A and at position 121 to A and
b) an amino acid substitution at position 217 of SEQ ID NO: 11 to S or
7) an Fc region comprising
a) an amino acid substitution at position 120 of SEQ ID NO: 11 to A and at position 121 to A and
b) an amino acid substitution at position 215 of SEQ ID NO: 11 to G or
8) an Fc region comprising
a) an amino acid substitution at position 119 of SEQ ID NO: 11 to G and
b) an amino acid substitution at position 156 of SEQ ID NO: 11 to N or
9) an Fc region comprising
a) an amino acid substitution at position 120 of SEQ ID NO: 11 to A and at position 121 to A;
b) an amino acid substitution at position 156 of SEQ ID NO: 11 to N and
c) an amino acid substitution at position 217 of SEQ ID NO: 11 to S.

In one embodiment, the Fc region includes the modifications as set out above but does not have any further modifications.

For example, in one embodiment of the above aspects, the invention relates to a canine, chimeric or caninised IgG-B polypeptide or Fc region thereof wherein said Fc region comprises
a) an amino acid substitution at position 120 of SEQ ID NO: 11 to S and
b) an amino acid substitution at position 211 of SEQ ID NO: 11 to H, an amino acid substitution at position 212 of SEQ ID NO: 11 to I and an amino acid substitution at position 213 of SEQ ID NO: 11 to G.

For example, in one embodiment of the above aspects, the invention relates to a canine, chimeric or caninised IgG-B polypeptide or Fc region thereof wherein said Fc region comprises
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G.

For example, in one embodiment of the above aspects, the invention relates to a canine, chimeric or caninised IgG-B polypeptide or Fc region thereof wherein said Fc region comprises
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 217 of SEQ ID NO: 11 to S.

For example, in one embodiment of the above aspects, the invention relates to a canine, chimeric or caninised IgG-B polypeptide or Fc region thereof wherein said Fc region comprises
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 215 of SEQ ID NO: 11 to G.

For example, in one embodiment of the above aspects, the invention relates to a canine, chimeric or caninised IgG-B polypeptide or Fc region thereof wherein said Fc region comprises an amino acid substitution at position 120 of SEQ ID NO: 11 to A and at position 121 to A.

For example, in one embodiment of the above aspects, the invention relates to a canine, chimeric or caninised IgG-B polypeptide or Fc region thereof wherein said Fc region comprises
a) an amino acid substitution at position 120 of SEQ ID NO: 11 to A and at position 121 to A and
b) an amino acid substitution at position 217 of SEQ ID NO: 11 to S.

For example, in one embodiment of the above aspects, the invention relates to a canine, chimeric or caninised IgG-B polypeptide or Fc region thereof wherein said Fc region comprises
a) an amino acid substitution at position 120 of SEQ ID NO: 11 to A and at position 121 to A and
b) an amino acid substitution at position 215 of SEQ ID NO: 11 to G.

For example, in one embodiment of the above aspects, the invention relates to a canine, chimeric or caninised IgG-B polypeptide or Fc region thereof wherein said Fc region comprises
a) an amino acid substitution at position 119 of SEQ ID NO: 11 to G and
b) an amino acid substitution at position 156 of SEQ ID NO: 11 to N.

For example, in one embodiment of the above aspects, the invention relates to a canine or caninised IgG-B polypeptide or Fc region thereof wherein said Fc region comprises
a) an amino acid substitution at position 120 of SEQ ID NO: 11 to A and at position 121 to A;
b) an amino acid substitution at position 156 of SEQ ID NO: 11 to N and
c) an amino acid substitution at position 217 of SEQ ID NO: 11 to S.

For example, the invention relates to a canine, chimeric or caninised IgG-B polypeptide or Fc region thereof wherein said polypeptide comprises SEQ ID No. 12, 13, 14, 15, 16, 17, 18, 19 or 20 or a sequence with at least 80%, 85%, 90% or 95% sequence identity thereto wherein the sequence is not a wild type sequence.

The invention also relates to a pharmaceutical composition comprising a canine, chimeric or caninised IgG-B polypeptide or Fc region thereof as described herein.

The invention also relates to a nucleic acid encoding a canine, chimeric or caninised IgG-B polypeptide or Fc region thereof as described herein.

The invention further relates to a vector comprising a canine, chimeric or caninised IgG-B nucleic acid or Fc region thereof as described above.

The invention further relates to a host cell comprising a canine, chimeric or caninised IgG-B nucleic acid as described above.

The invention further relates to a canine, chimeric or caninised IgG-B polypeptide or Fc region as described herein or a pharmaceutical composition comprising the canine, chimeric or caninised IgG-B polypeptide or Fc region as described herein for use in the treatment of disease, for example an inflammatory or autoimmune disease.

The invention further relates to a method of treating a disease in a subject comprising an effective amount of the polypeptide or pharmaceutical composition comprising the canine, chimeric or caninised IgG-B polypeptide or Fc region thereof as described above to said subject. The disease may be an inflammatory or autoimmune disease.

The invention further relates to a kit comprising the comprising the canine, chimeric or caninised IgG-B polypeptide or Fc region thereof as described herein or a pharmaceutical composition as described herein.

The invention further relates to an in vitro or in vivo method for repressing effector function comprising contacting a cell or tissue with a polypeptide or canine, chimeric or caninised IgG-B polypeptide or Fc region thereof as described herein.

With reference to wild type feline IgG1 constant region sequence as shown in SEQ ID NO. 31, the lower hinge comprises residues 119-125, the proline region comprises residues 211-217 and the SHED region comprises residues 151-156. FIG. 15A illustrates the domains and the residue numbering. Feline variant sequences according to the invention in the lower hinge, proline region and SHED region compared to wild type of SEQ ID NO. 31 (IgG1) are shown. The numbering of residues is with reference to SEQ ID NO:31 (FIG. 15B) which shows the feline IgG1 wild type constant region amino acid sequence. FIG. 13A shows full feline IgG isoform wild type sequences.

Thus, in another aspect, the invention relates to a feline or felinised IgG1 polypeptide or Fc region thereof comprising a modified Fc region wherein said Fc region comprises
a) an amino acid substitution at position 120 of SEQ ID NO: 31 to A and an amino acid substitution at position 121 of SEQ ID NO: 31 to A or an amino acid substitution at position 120 of SEQ ID NO: 31 to I and an amino acid substitution at position 121 of SEQ ID NO: 31 to P and/or
b) one or more of the following modifications: an amino acid substitution at position 217 of SEQ ID NO: 31 to S or A, an amino acid substitution at position 215 of SEQ ID NO: 31 to G, and/or an amino acid substitution at position 123 of SEQ ID NO: 31 to A.

In one embodiment, the feline or felinised IgG1 polypeptide or Fc region thereof comprises a modified Fc region wherein said Fc region comprises
a) an amino acid substitution at position 120 of SEQ ID NO: 31 to A, an amino acid substitution at position 121 of SEQ ID NO: 31 to A and further comprising either an amino acid substitution at position 217 of SEQ ID NO: 31 to S or an amino acid substitution at position 215 of SEQ ID NO: 31 to G or
b) an amino acid substitution at position 120 of SEQ ID NO: 31 to I, an amino acid substitution at position 121 of SEQ ID NO: 31 to P and further comprising either an amino acid substitution at position 217 of SEQ ID NO: 31 to A or an amino acid substitution at position 123 of SEQ ID NO: 31 to A.
   In one embodiment, the feline or felinised IgG1 polypeptide or Fc region thereof comprises a modified Fc region wherein said Fc region comprises an amino acid substitution at position 120 of SEQ ID NO: 31 to A, an amino acid substitution at position 121 of SEQ ID NO: 31 to A and an amino acid substitution at position 217 of SEQ ID NO: 31 to S.

In one embodiment, the feline or felinised IgG1 polypeptide or Fc region thereof comprises an amino acid substitution at position 120 of SEQ ID NO: 31 to A, an amino acid substitution at position 121 of SEQ ID NO: 31 to A and an amino acid substitution at position 215 of SEQ ID NO: 31 to G.

In one embodiment, the feline or felinised IgG1 polypeptide or Fc region thereof comprises an amino acid substitution at position 120 of SEQ ID NO: 31 to I, an amino acid substitution at position 121 of SEQ ID NO: 31 to P and an amino acid substitution at position 123 of SEQ ID NO: 31 to A and optionally an amino acid substitution at position 217 of SEQ ID NO: 31 to A.

In one embodiment, the feline or felinised IgG1 polypeptide or Fc region thereof comprises an amino acid substitution at position 120 of SEQ ID NO: 31 to I, an amino acid substitution at position 121 of SEQ ID NO: 31 to P and an amino acid substitution at position 217 of SEQ ID NO: 31 to A.

In one embodiment, the polypeptide has reduced Fc-mediated effector function when compared to the same polypeptide comprising a wild-type IgG1 Fc domain.

In one embodiment, the effector function is antibody-dependent cell-mediated cytotoxicity (ADCC).

In one embodiment, the effector function is complement-dependent cytotoxicity (CDC) or antibody-dependent cell-mediated phagocytosis (ADCP).

In one embodiment, the polypeptide has lower affinity for an Fc gamma receptor (FcγR) when compared to the same polypeptide comprising a wild-type IgG1 Fc domain.

In one embodiment, the polypeptide or Fc region retains FcRn binding.

The invention also relates to a pharmaceutical composition comprising a feline or felinised polypeptide or Fc region thereof as described above.

The invention also relates to an in vitro, ex vivo or in vivo method for repressing effector function comprising contacting a cell or tissue with a feline or felinised polypeptide or Fc region thereof as described above.

The invention further relates to a c feline or felinised polypeptide or Fc region thereof as described herein or a pharmaceutical composition comprising the feline or felinised polypeptide or Fc region thereof as described herein for use in the treatment of disease, for example an inflammatory or autoimmune disease.

The invention further relates to a method of treating a disease in a subject comprising an effective amount of the polypeptide or pharmaceutical composition comprising the feline or felinised polypeptide or Fc region thereof as described above to said subject. The disease may be an inflammatory or autoimmune disease.

In one embodiment, the effector function is antibody-dependent cell-mediated cytotoxicity (ADCC).

In one embodiment, the effector function is complement-dependent cytotoxicity (CDC).

The invention also relates to nucleic acid encoding a feline or felinised polypeptide or Fc region thereof as described above.

The invention also relates to vector comprising a feline or felinised nucleic acid as described above

The invention also relates to host cell comprising a feline or felinised nucleic acid or a vector as described above.

The invention also relates to kit comprising a feline or felinised polypeptide or Fc region thereof or a pharmaceutical composition as described above.

The invention also relates to isolated IgG3 feline polypeptide comprising SEQ ID. 33 or an Fc region thereof.

The invention also relates to isolated IgG3 feline polynucleotide encoding the polypeptide or an Fc region as above.

The invention also relates to isolated recombinant antibody comprising the polypeptide or an Fc region as above.

The invention is further illustrated in the following non-limiting figures.

### DESCRIPTION OF FIGURES

FIG. 1 A, shows amino acid sequences of wild type canine IgG (cIg-G) IgG-A (SEQ ID NO. 22) (a), IgG-B (SEQ ID NO. 21) (b), IgG-C(SEQ ID NO. 23) (c) and IgG-D (d) (SEQ ID NO. 24). The constant region begins at residue 140. Highlighting identifies the lower hinge, proline sandwich and SHED areas. B, shows wild-type canine chimeric (cIg-G) IgG-A (SEQ ID NO. 22) (a), IgG-B (SEQ ID NO. 21) (b), IgG-C(SEQ ID NO. 23) (c) and IgG-D (d) (SEQ ID NO. 24) with Ofatumumab variable regions as visualised via non-reducing SDS-PAGE. Note that IgG-A and IgG-D exhibits hinge instability and HL half antibody species is visible.

FIG. 2 A, shows amino acid sequences of wild type canine IgG-A, -B, -C and -D and Def IgG-B “def 1-9” as described herein. Mutations are in the lower hinge (amino acid residues 119-125), proline sandwich (211-217) and ‘SHED’ areas (151-156). In bold are amino acids that are mutated in Def mutants compared to wild type canine IgG-B (cIgG-B). The numbering of residues is with reference to SEQ ID NO:11 which shows the canine IgG-B wild type constant region amino acid sequence. This sequence is shown in FIG. 2B. With reference to FIG. 2A, the lower hinge of IgG-A, -B, -C, -D is SEQ ID NO: 48, 49, 50, 51 respectively. The proline sandwich of IgG-A, -B, -C, -D is SEQ ID NO: 52, 53, 54, 55 respectively. The SHED area of IgG-A, —B, —C, -D is SEQ ID NO: 56, 57, 58, 59 respectively. The lower hinge of B-def1, B-def2, B-def3, B-def4, B-def5, B-def6, B-def7, B-def8, B-def9 is SEQ ID NO: 60, 61, 62, 63, 64, 65, 66, 67, 68 respectively. The proline sandwich of B-def1, B-def2, B-def3, B-def4, B-def5, B-def6, B-def7, B-def8, B-def9 is SEQ ID NO: 69, 70, 71, 72, 73, 74, 75, 76, 77 respectively. The SHED area of B-def1, B-def2, B-def3, B-def4, B-def5, B-def6, B-def7, B-def8, B-def9 is SEQ ID NO: 78, 79, 80, 81, 82, 83, 84, 85, 86 respectively

FIG. 3 shows CDC activity of IgG-B mutants: All Def mutants repress IgG-B CDC activity. Complement-dependent cytotoxicity assay showing the reduced complement dependent killing of canine T cells expressing human CD20 (CLBL1 hCD20) by effector function deficient IgG-B mutants Def 1, 2, 3, 5, 6, 7, 8 and 9, when compared to wild type (WT) canine IgG-B. All antibodies used in this assay have Ofatumumab variable regions. Data are plotted as percentage of killing where 100% means all cells are killed and 0% means signal was identical to what obtained in control cells (no antibody added).

FIG. 4 shows a comparison of ADCC activity for chimeric Ofatumumab IgG-B and Rituximab IgG-B, demonstrating the variable region sequence does not also contribute to potency of ADCC activity

FIG. 5 shows ADCC activity of IgG-B mutants on wild type or hCD20 expressing MDCK II cells. Antibody-dependent cell-mediated cytotoxicity assay showing the reduced ability of Def mutants 1, 2, 3, 5, 6, 7, 8 and 9 canine IgG-B with Ofatumumab variable regions to induce cellular dependent killing of canine cells expressing human CD20 when compared to wild type (WT) canine IgG-B. Data are plotted as percentage of killing where 100% means all cells are killed and 0% means signal was identical to what obtained in control cells (no antibody added). FIG. 5 A shows graphs for individual Def mutants. FIG. 5 B is a combination of the data shown in FIG. 5 A. The top panel shows the results obtained for the mutants using human CD20 MDCK cells and the bottom panel shows the results using wild type MDCK cells.

FIG. 6 shows ADCC activity of Def 2, 3 and 7 mutants: the effector deficient mutants, Def2, Def3 and Def7, completely abolish ADCC function in a wide dose range compared to IgG-B wild-type (effector enabled) counterparts. Direct comparison of canine IgG-B WT with Def2, Def3 and Def7 mutant with Ofatumumab variable region ability to induce antibody-dependent cell-mediated cytotoxicity of canine cells expressing human CD20 at different antibody concentrations. Data are plotted as percentage of killing where 100% means all cells are killed and 0% means signal was identical to what obtained in control cells (no antibody added).

FIG. 7 shows results of the accelerated stability test (A) and protein A binding (B). A, Both WT and all canine Def mutants showed no significant reduction in % as well as area of monomeric peak at 4° C. after 14 days of storage (not shown). Moreover, all def mutants showed higher % and similar area of monomeric peak to WT after 14 days incubated at 40° C., meaning that overall protein stability is not affected by deficient mutants. B, Both kinetics and steady state affinity analyses showed no statistical difference in binding affinity between IgG-Bwt and def mutants to Protein A and FcRn.

FIG. 8 shows FcγR1 binding capacity of Ofatumumab IgG-B WT vs Def mutants. SPR measurements of Ofatumumab IgG-B WT and Def mutants 1 to 8 showed statistically significant differences in binding affinity to FcγR1. It is worth noting that there is a good correlation between SPR and ADCC data of these molecules (WT>Def5-8>Def1,2,3,6,7), indicating that higher affinity to FcγR1 correspond to higher killing % in in vitro assays.

FIG. 9 shows in vivo PK obtained by injecting a single dose of 1 mg/kg of Ofa-IgGB WT, Def2, Def3 or Def7 in Rag1 KO mice. Background levels of detection (obtained by pre injection sera) are indicated by the dotted lines. Def2,3,7 mutants have similar pharmacokinetics profile compared to WT IgG-B WT.

FIG. 10 shows DLS measurements of canine Def mutants. All IgG-B mutants showed radius similar to wild type IgG-B. This indicates that mutations introduced didn't altered the stability of IgG-B Fc domain.

FIG. 11 shows Tonset measurements of canine Def mutants compared to WT. All IgG-B mutants showed similar Tonset to wild type IgG-B. This indicates that aggregation propensity caused by temperature stress is similar in all deficient mutants in comparison to IgG-B Fc WT domain.

FIG. 12 shows the deduced gene structure of feline IGHG3 as confirmed by Sanger sequencing of a PCR product (1869 bp) generated using FACB-160A15 commercial BAC library available at Texas A&M University as a template.

FIG. 13 A, shows amino acid sequences of wild type feline IgG (fIgG) 1 (SEQ ID NO. 31) (a), 2 (SEQ ID NO. 32) (b) and 3 (SEQ ID NO. 33) (c). The constant region begins at residue 141. Highlighting identifies the lower hinge, proline sandwich and SHED areas. (B) shows wild-type feline chimeric fIgG1 (SEQ ID NO. 31) (a), 2 (SEQ ID NO. 32) (b) and 3 (SEQ ID NO. 33) with Ofatumumab variable regions as visualised via non-reducing SDS-PAGE. Noted that IgG2 has hinge instability resulting in HL half antibody band migrating at lower molecular weight around 75 kDa.

FIG. 14 shows ADCC activity of feline WT IgG1, 2 and 3. Antibody-dependent cell-mediated cytotoxicity assay showing the reduced ability of WT IgG2 and 3 with Ofatumumab variable regions to induce cellular dependent killing of canine cells expressing human CD20 when compared to wild type (WT) feline IgG 1. WT IgG1 targeting a different antigen other than CD20 is shown as a negative control. Data are plotted as percentage of killing where 100% means all cells are killed and 0% means signal was identical to what obtained in control cells (no antibody added).

FIG. 15 shows amino acid sequences of wild type feline IgG-1, 2 and 3 and Def IgG1 “Def 1-4” as described herein. Mutations are in the lower hinge (amino acid residues 119-125), proline sandwich (211-217) and ‘SHED’ areas (151-156). In bold are highlighted amino acids that are mutated in Def mutants compared to wild type feline IgG-1 (fIgG-1). The numbering of residues is with reference to SEQ ID NO:31 which shows the feline IgG-1 wild type constant region amino acid sequence. This sequence is shown in FIG. 15B. With reference to FIG. 15A, the lower hinge of IgG-1, -2, -3 is SEQ ID NO: 87, 88, 89 respectively. The proline sandwich of IgG-1, -2, -3 is SEQ ID NO: 90, 91, 92 respectively. The SHED area of IgG-1, -2, -3 is SEQ ID NO: 93, 94, 95 respectively. The lower hinge of 1-def1, 1-def2, 1-def3, 1-def4 is SEQ ID NO: 96, 97, 98, 99 respectively. The proline sandwich of 1-def1, 1-def2, 1-def3, 1-def4 is SEQ ID NO: 100, 101, 102, 103 respectively. The SHED area of 1-def1, 1-def2, 1-def3, 1-def4 is SEQ ID NO: 104, 105, 106, 107 respectively.

FIG. 16 shows ADCC activity of feline Def 1, 2, 3 and 4 mutants compared to WT. Effector deficient mutant Def3 completely abolishes ADCC function compared to wild-type (effector enabled) counterparts. Direct comparison of canine IgG1 WT with Def 1, 2, 3 and 4 mutants with Ofatumumab variable region ability to induce antibody-dependent cell-mediated cytotoxicity of canine cells expressing human CD20 at different antibody concentrations. The results show the complete abolishment of ADCC activity of feline IgG1 by mutations present in the Def3 variant. Data are plotted as percentage of killing where 100% means all cells are killed and 0% means signal was identical to what obtained in control cells (no antibody added).

FIG. 17 shows native SDS-Page images of all feline Def mutants, which show no clear difference from that of WT (A). All def mutants showed no significant reduction in % as well as area of monomeric peak as determined by HPLC-SEC (B), meaning that the overall protein stability is not affected introduced mutations in Def mutants.

FIG. 18 SPR measurements of both kinetics and steady state affinity analyses of Ofatumumab IgG1 WT and Def mutants 1 to 4 showed no statistical difference in binding affinity to Protein A (A) and FcRn (B). SPR measurements of Ofatumumab IgG1 WT and Def mutants 1 to 4 showed significant differences in binding affinity to FcγR1, with def mutants 1 to 3 showing similar binding as to that of wild type fIgG2 and 3 (C).

FIG. 19 A) shows DLS measurements of feline Def mutants. All IgG1 mutants showed diameter similar to wild type IgG1. This indicates that mutations introduced do not altered the stability and aggregation propernsity of IgG1 Fc domain. B) shows Tonset measurements of Def mutants. All IgG1 mutants showed similar Tonset to wild type IgG1. This indicates that aggregation propensity caused by temperature stress is similar in all deficient mutants in comparison to IgG1 Fc WT domain.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols. 1 and 2, Ontermann and Duebel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The invention provides biological therapeutics for veterinary use, in particular antibody-based therapeutics for use in the treatment of dogs.

The term “antibody” as used herein refers to any immunoglobulin (Ig) molecule, or antigen binding portion or fragment thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. As used herein, the term “antibody” encompasses not only intact polyclonal or monoclonal antibodies.

In a full-length antibody, each heavy chain is comprised of a heavy chain variable region or domain (abbreviated herein as HCVR) and a heavy chain constant region. The heavy chain constant region is comprised of three constant heavy chain domains, CH1, CH2 and CH3. Each light chain is comprised of a light chain variable region or domain (abbreviated herein as LCVR) and a light chain constant region. The light chain constant region is comprised of one domain, CL.

Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies. The constant domains are not involved directly in the binding of antibody to an antigen, but exhibit various effector functions, such as participation of the antibody in antibody-dependent cellular toxicity.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can generally be of any isotype, class or subclass. The CH3 domain according to the various aspects of the invention is a CH3 domain of the canine IgG subtype, for example IgG-A, IgG-B, IgG-C, and IgG-D. According to other aspects of the invention, the CH3 domain is the CH3 domain of the feline IgG subtype, for example IgG1 (SEQ ID NO: 31), IgG2 (SEQ ID NO: 32) and IgG3 (SEQ ID NO: 33).

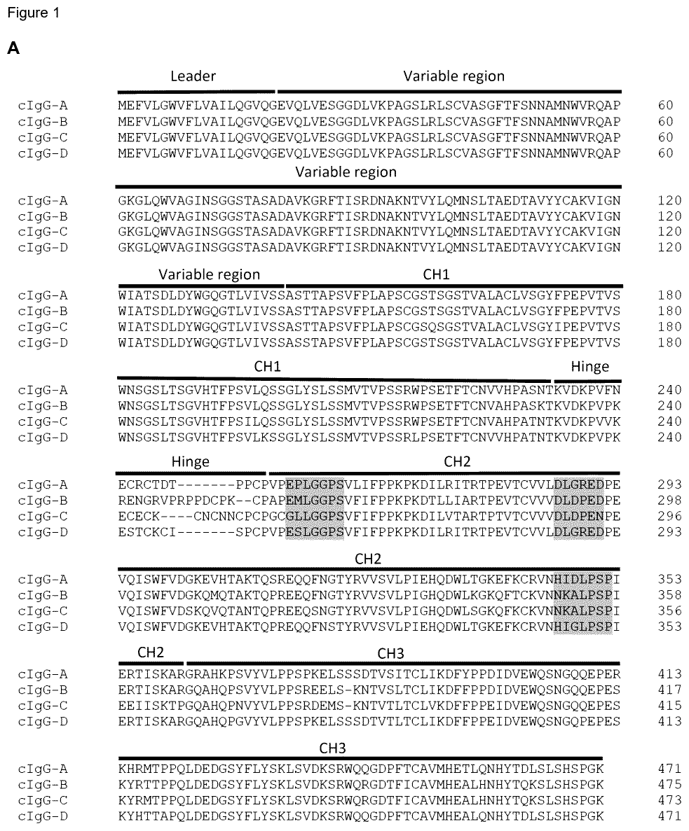

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). Exemplary amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Amino acid sequences for canine IgG-A, IgG-B, IgG-C and IgG-D as used by the inventors and according to the aspects and embodiments of the invention are shown in FIG. 1 and Table 1 (SEQ ID Nos. 22, 21, 23 and 24).

Canine antibodies also contain two types of light chains, kappa and lambda (GenBank accession number kappa light chain amino acid sequence ABY 57289.1, GenBank accession number ABY 55569.1).

In canine, there are four IgG heavy chain isoforms which are referred to as IgG-A, IgG-B, IgG-C, and IgG-D. The DNA and amino acid sequences of these four heavy chains were first identified by Tang et al. (Vet. Immunol. Immunopathol. 80: 259-270 (2001)). Exemplary amino acid and DNA sequences for these heavy chains are also available from the GenBank data bases (IgG-A: accession number AAL35301.1, IgG-B: accession number AAL35302.1, IgG-C: accession number AAL35303.1, IgG-D: accession number AAL35304.1). Amino acid sequences for canine IgG-A, IgG-B, IgG-C and IgG-D as used by the inventors and according to the aspects and embodiments of the invention are shown in FIG. 1 and Table 1 (SEQ ID Nos. 22, 21, 23 and 24).

Canine antibodies also contain two types of light chains, kappa and lambda (GenBank accession number kappa light chain amino acid sequence ABY57289.1, GenBank accession number ABY55569.1).

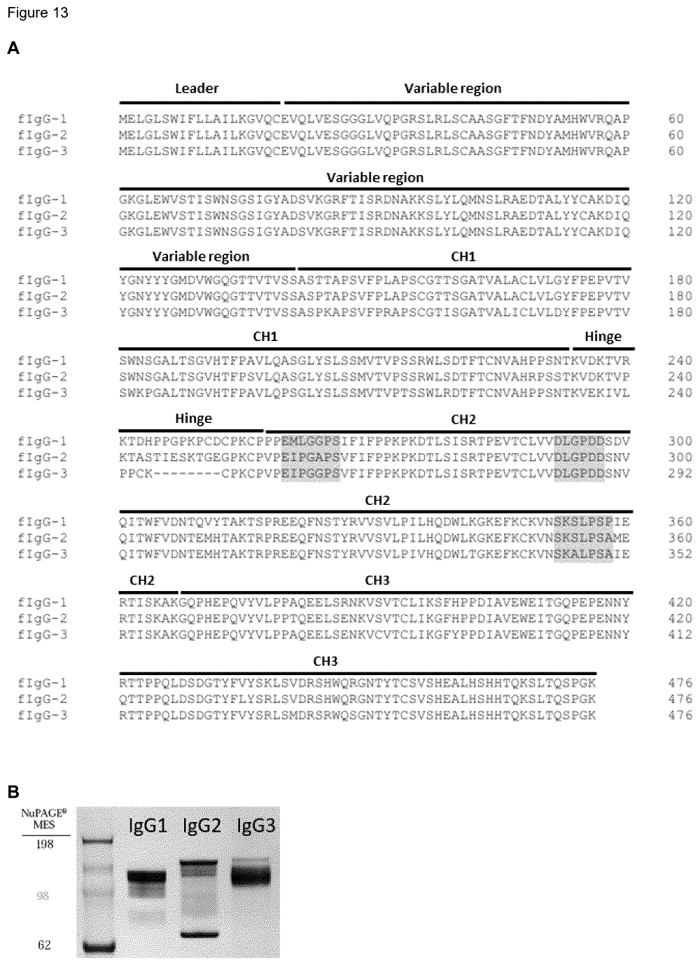

In Feline, two variants of IgG1 have been first identified by Kanai et al (Vet. Immunol. Immunopathol. 73: 53-62 (2001)) and referred to as IgG1a and IgG1b. The second IgG has been identified by Strietzel et al (Vet. Immunol. Immunopathol. 158: 214-223 (2014)), and referred to as IgG2, GenBank accession number KF811175.1. These isoforms were identified from transcribed RNA sequences by RACE. The dominant expressed isoform is IgG1, which accounts for over 90% expressed IgG in feline PBMC (Lu et al Scientific Reports 7:12713 (2017). The inventors have identified a third feline IgG, referred to as IgG3 by annotating the genomic locus of feline IgGH region. Amino acid sequences for feline IgG1, IgG2, IgG3 as used by the inventors and according to the aspects and embodiments of the invention are shown in FIG. 13 and Table 1 (SEQ ID Nos. 31, 32, and 33).

Thus, aspects of the invention also relate to an isolated polypeptide comprising SEQ ID NO. 33 or a portion thereof, an isolated recombinant antibody comprising SEQ ID NO. 33 or a portion thereof, methods for producing a recombinant antibody using SEQ ID NO. 33 and the use of SEQ ID NO. 33 in methods for producing a recombinant antibody.

Feline antibodies also contain two types of light chain, kappa and lambda. Kappa constant chain sequence was first identified by Weber at al in 1999 with GenBank accession number AF 198257.1. Lambda constant region of feline is poorly defined, and so far two variants have been described with GenBank accession number XM_003994910.1 and E07339.1. High variability in detected cDNA sequences outside the CDR regions from feline PBMC RNA samples to these two published lambda sequences suggest that more allotypes exist in cat and are undefined (Strietzel et al 2014 and Lu et al 2017).

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are one of the most commonly used numbering systems for human antibodies (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion.

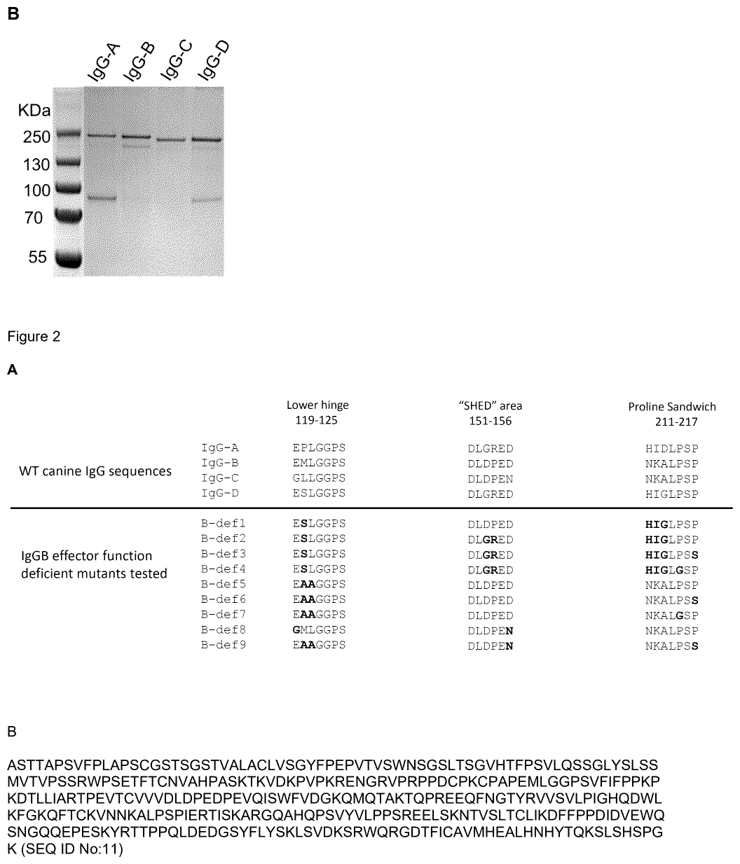

Unless otherwise stated, the numbering used herein when referring to canine residues at certain positions is with reference to the IgG-B constant region as shown SEQ ID NO. 11 (see also FIG. 2). By “position” herein is thus meant a location in the sequence of a protein, e.g. with reference to FIG. 1. Corresponding positions are determined as outlined, generally through alignment with other wild-type sequences. By “residue”, e.g. amino acid residue, as used herein is meant a position in a protein and its associated amino acid identity. For example, glutamic acid is the residue at position 119 of the constant region of canine IgG-B with reference to SEQ ID NO:11.

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

Of particular interest in the present invention are the Fc regions. By “Fc” or “Fc region” or “Fc domain” as used herein is meant the polypeptide comprising the constant region of an antibody excluding the first constant region immunoglobulin domain and, in some cases, part of the hinge.

In human, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat. Canine isoforms are shown in FIG. 1, these include the Fc region. Canine constant region of IgG-B, including Fc, is shown in SEQ ID NO. 11 and FIG. 2B.

Fc as used herein may refer to this region in isolation, or this region in the context of an Fc fusion (“fusion composition” or “fusion construct”), as described herein. Fc domains include all or part of an Fc region; that is, N- or C-terminal sequences may be removed from wild-type or variant Fc domains recited herein, as long as this does not affect function.

Briefly, IgG functions are generally achieved via interaction between the Fc region of the Ig and an Fcγ receptor (FcγR) or another binding molecule, sometimes on an effector cell. This can trigger the effector cells to kill target cells to which the antibodies are bound through their variable (V) regions. Also, antibodies directed against soluble antigens might form immune complexes which are targeted to FcγRs which result in the uptake (opsonisation) of the immune complexes or in the triggering of the effector cells and the release of cytokines.

By “Fc gamma receptor”, “FcγR” or “FcgammaR” as used herein is meant any member of the family of proteins that bind the IgG antibody Fc region and is encoded by an FcγR gene.

In humans, three classes of FcγR have been characterised, although the situation is further complicated by the occurrence of multiple receptor forms. The three classes are:
(i) FcγRI (CD64) including isoforms FcγRIa, FcγRIb, and FcγRIc binds monomeric IgG with high affinity and is expressed on macrophages, monocytes, and sometimes neutrophils and eosinophils;
(ii) FcγRII (CD32) binds complexed IgG with medium to low affinity and is widely expressed. These receptors can be divided into two important types, FcγRIIa and FcγRIIb. The ‘a’ form of the receptor is found on many cells involved in killing (e. g. macrophages, monocytes, neutrophils) and seems able to activate the killing process and occurs as two alternative alleles. The ‘b’ form seems to play a role in inhibitory processes and is found on B-cells, macrophages and on mast cells and eosinophils. On B-cells it seems to function to suppress further immunoglobulin production and isotype switching to for example, the IgE class. On macrophages, the b form acts to inhibit phagocytosis as mediated through FcγRIIa. On eosinophils and mast cells the b form may help to suppress activation of these cells through IgE binding to its separate receptor and
(iii) FcγRIII (CD16) binds IgG with medium to low affinity and exists as two types. FcγRIIIa is found on NK cells, macrophages, eosinophils and some monocytes and T cells and mediates ADCC. FcγRIIIb is highly expressed on neutrophils. Both types have different allotypic forms.

Canine Fc receptors are described in Bergeron et al L. M. Bergeron et al.; Veterinary Immunology and Immunopathology 157 (2014) 31-41. Canine has RI, RIIb, RIII, but not Riia.

Canine Fc receptors are described in Bergeron et al L. M. Bergeron et al.; Veterinary Immunology and Immunopathology 157 (2014) 31-41. Canine has RI, RIIb, RIII, but not Riia.

Feline Fc receptors are described in Kanai et al; Veterinary Immunology and Immunopathology 73 (2000) 53-62, Strietzel et al; Veterinary Immunology and Immunopathology 158 (2014) 214-223, and Lu et al; scientific Reports 7 (2017) 12713.

As well as binding to FcγRs, IgG antibodies can activate complement and this can also result in cell lysis, opsonisation or cytokine release and inflammation. The Fc region also mediates such properties as the transportation of IgGs to the neonate (via the so-called “FcRn”), increased half-life (also believed to be effected via an FcRn-type receptor) and self-aggregation. The Fc-region is also responsible for the interaction with protein A and protein G (which interaction appears to be analogous to the binding of FcRn).

By “effector function” as used herein is meant a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP) and complement-dependent cytotoxicity (CDC).

Antigen binding or other fragments are also contemplated according to the aspects and embodiments of the invention. Antigen binding fragments include, for example, Fab, Fab′, F(ab′)2, Fd, Fv, single domain antibodies (sdAbs), e.g. VH single domain antibodies, fragments including complementarity determining regions (CDRs), single chain variable fragment antibodies (scFv), maxibodies, minibodies, intrabodies, diabodies, triabodies, tetrabodies, and bis-scFv, and polypeptides that contain at least a portion of an immunoglobulin that is sufficient to confer specific antigen binding to the polypeptide. Fragments according to the invention may include the Fc domain or a part thereof or the constant region. Thus, a constant region comprising a modified Fc polypeptide as described herein is also within the scope of the invention.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site. “Single-chain Fv” also abbreviated as “sFv” or “scFv” are antibody fragments that comprise the VH and VL antibody domains connected into a single polypeptide chain.

The term “antigen binding site” refers to the part of the antibody or antibody fragment that comprises the area that specifically binds to an antigen. An antigen binding site may be provided by one or more antibody variable domains. Preferably, an antigen binding site is comprised within the associated VH and VL of an antibody or antibody fragment.

A “chimeric antibody” is a recombinant protein that contains the variable domains including the complementarity determining regions (CDRs) of an antibody derived from one species, while the constant domains of the antibody molecule are derived from those of another species, e.g. a canine antibody. An exemplary chimeric antibody is a chimeric human-canine antibody.

A “humanized antibody” is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. In certain embodiments, a limited number of framework region amino acid residues from the parent (rodent) antibody may be substituted into the human antibody framework region sequences.

As used herein, the term “caninized antibody” or a “felinized antibody” refers to forms of recombinant antibodies that contain sequences from both canine and non-canine (e.g., murine) antibodies or from both feline and non-feline (e.g. murine) antibodies, respectively. In general, the caninized antibody will comprise substantially all of at least one or more typically, two variable domains in which all or substantially all of the hypervariable loops correspond to those of a non-canine (or non-feline) immunoglobulin, and all or substantially all of the framework (FR) regions (and typically all or substantially all of the remaining frame) are those of a canine (or feline) immunoglobulin sequence. A caninized or felinized antibody may comprise both the three heavy chain CDRs and the three light chain CDRS from a murine or human antibody together with a canine or feline frame or a modified canine or feline frame. A modified canine or feline frame comprises one or more amino acids changes that can further optimize the effectiveness of the caninized or felinized antibody, e.g., to increase its binding to its target.

A “speciated” antibody (e.g. humanized, caninized, felinized, chimeric) is one which has been engineered to render it similar to antibodies of the target species. In one embodiment, a “speciated” antibody is greater than about 80%, 85% or 90% similar to antibodies of the target species. Such speciated antibodies are within the scope of the invention.

In a preferred embodiment, the antibody or antibody fragment is fully canine.

In contrast to speciated antibodies, fully canine or feline antibodies of the present invention have canine or feline variable regions and do not include full or partial CDRs or FRs from another species. Advantageously, fully canine or feline antibodies as described herein have been obtained from transgenic mice comprising canine or feline immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully canine or feline antibodies produced in this way generate antibodies with optimal properties for developability, minimizing lengthy lead optimization prior to production at scale. Advantageously, such fully canine or feline antibodies present the lowest possible risk of immunogenicity when introduced into a patient animal which, in turn, facilitates a repeated dosing regimen. Adverse in vivo immunogenicity can be assessed, for example, by assays to identify the production of anti-drug antibodies (ADA), or a loss of efficacy over time in vivo. Given that ex vivo mAb engineering runs the risk of introducing development liabilities, immunogenicity, and reduced affinity (as outlined above), fully canine or feline antibodies of the present invention are, therefore, most likely to be efficacious therapies in a clinical context.

The term “monoclonal antibody” as used herein refers to an antibody derived from a single B or plasma cell. All antibody molecules in a monoclonal antibody preparation are identical except for possible naturally occurring post-translation modifications (e.g., isomerizations, amidations, carbohydrate addition) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

The term “epitope” or “antigenic determinant” refers to a site on the surface of an antigen (to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping) are well known in the art and include, for example, immunoblotting and immunoprecipitation assays, wherein overlapping or contiguous peptides from are tested for reactivity with a given antibody or antibody fragment. An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody.

The term “isolated” protein or polypeptide refers to a protein or polypeptide that is substantially free of other proteins or polypeptides, having different antigenic specificities. Moreover, protein or polypeptide may be substantially free of other cellular material and/or chemicals. Thus, the protein, nucleic acids and polypeptides described herein are preferably isolated. Thus, as used herein, an “isolated” protein, or polypeptide means protein or polypeptide that has been identified and separated and/or recovered from a component of its natural cell culture environment. Contaminant components of its natural environment are materials that would interfere with diagnostic or therapeutic uses for the protein or polypeptide, and may include enzymes, hormones, and other proteinaceous or non-proteinaceous solutes.

By “amino acid” herein is meant one of the 20 naturally occurring amino acids or any non-natural analogues that may be present at a specific, defined position. Amino acid encompasses both naturally occurring and synthetic amino acids. Although in most cases, when the protein is to be produced recombinantly, only naturally occurring amino acids are used.

The present invention provides proteins comprising variant Fc regions. By “protein” herein is meant at least two covalently attached amino acids, which includes proteins, polypeptides, oligopeptides and peptides. The protein may be made up of naturally occurring amino acids and peptide bonds, or synthetic peptidomimetic structures, for example analogs such as peptoids. Again, when the protein is to be produced recombinantly, only naturally occurring amino acids are used.

By “variant” or “mutant” herein is meant a polypeptide sequence that differs from that of a wild-type sequence by virtue of at least one amino acid modification. As used herein, a “substitution of an amino acid residue” with another amino acid residue in an amino acid sequence of a protein or polypeptide as described herein, is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence.

Amino acid modifications in general refer include substitutions, insertions and deletions, with the former being preferred in many cases. The variants of the invention include amino acid substitutions in the Fc domain, and they can include any number of further modifications, as long as the function of the protein is still present, as described herein. However, in general, from 1, 2, 3, 4, 5, 6, 7, 8, 9 or 10 modifications in addition to the Fc modifications are generally utilized as often the goal is to alter function with a minimal number of modifications. In some cases, there are from 1 to 5 modifications, with from 1-2, 1-3 and 1-4 also finding use in many embodiments. It should be noted that the number of amino acid modifications may be within functional domains: for example, it may be desirable to have from 1-6 modifications in the Fc region of wild-type or engineered proteins, as well as from 1 to 5 modifications in the Fv region, for example. A variant polypeptide sequence will preferably possess at least about 80%, 85%, 90%, 95% or up to 98% or 99% identity to the wild-type sequences or the parent sequences. It should be noted that depending on the size of the sequence, the percent identity will depend on the number of amino acids.

By “protein variant” or “variant protein” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent. The variant polypeptide sequence herein will preferably possess at least about 80% identity with a parent polypeptide sequence, and most preferably at least about 90% identity, more preferably at least about 95% identity.

By “parent polypeptide”, “parent protein”, “precursor polypeptide”, or “precursor protein” as used herein is meant an unmodified polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

Accordingly, by “parent Fc polypeptide” as used herein is meant an Fc polypeptide that is modified to generate a variant, and by “parent antibody” as used herein is meant an antibody that is modified to generate a variant antibody. Unless otherwise specified, the parent polypeptide includes a wild type IgG-B Fc domain e.g. as shown in the constant region amino acid sequence SEQ ID NO:11.

By “wild type” or “WT”, “wt” or “native” herein is meant an amino acid sequence or a nucleotide sequence that is found in nature, including allelic variations. A WT protein, polypeptide, antibody, immunoglobulin, IgG, etc. has an amino acid sequence or a nucleotide sequence that has not been intentionally modified. Canine wild type IgGs are shown in FIG. 1. Canine wild type constant region of IgG-B is shown in FIG. 2B. Feline wild type IgGs are shown in FIG. 13A. Feline wild type constant region of IgG1 is shown in FIG. 15B.

By “variant or mutant Fc region” herein is meant an Fc sequence that differs from that of a wild-type Fc sequence by virtue of at least one amino acid modification. Suitable substitutions are described herein. An Fc variant may only encompass an Fc region, or may exist in the context of an antibody, antibody fragment, Fc fusion, isolated Fc, Fc fragment, or other polypeptide that is substantially encoded by Fc. Fc variant may refer to the Fc polypeptide itself, compositions comprising the Fc variant polypeptide, or the amino acid sequence. Canine variant sequences according to the invention with modifications in the lower hinge, proline region and SHED region compared to wild type are shown in FIG. 2A. The numbering of residues is with reference to SEQ ID NO:11 which shows the canine IgG-B wild type constant region amino acid sequence. For feline Fc, the numbering is with reference to IgG1 (SEQ ID NO. 33).

An Fc variant comprises one or more amino acid modifications relative to a wild-type Fc polypeptide, wherein the amino acid modification(s) provide one or more optimized properties. An amino acid modification can be an amino acid substitution, insertion, or deletion in a polypeptide sequence, with multiple modifications being independently selected from these. By “amino acid substitution” or “substitution” herein is meant the replacement of an amino acid at a particular position in a parent polypeptide sequence with another amino acid. For example, the substitution E119G refers to a variant polypeptide, in which the glutamic acid at position 119 in IgG-B is replaced with glycine. The numbering is with reference to SEQ ID NO: 11. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

The Fc variants disclosed herein may have more than one amino acid modification as compared to the parent, for example from about one to fifty amino acid modifications, e.g., from about 1, 2, 3, 4, 5, 6, 7, 8, 9, or 10 amino acid modifications, from about one to about five amino acid modifications, etc. compared to the parent. The variant Fc variant sequences herein will possess about 80% identity with the parent Fc variant sequence, e.g., at least about 90% identity, at least about 95% identity, at least about 98% identity, at least about 99% identity, etc. Modifications disclosed herein also include glycoform modifications and other post-translational modifications as described below. Modifications may be made genetically using molecular biology or may be made enzymatically or chemically.

Among the many platforms for creating bsAbs, controlled Fab-arm exchange (cFAE) has proven useful based on minimal changes to native Ab structure and the simplicity with which bsAbs can be formed from two parental Abs (Labrijn et al, (2013) Efficient generation of stable bispecific IgG1 by controlled Fab-arm exchange. Proc. Natl. Acad. Sci. U.S.A. 110, 5145-5150). Controlled Fab-arm exchange uses a minimal set of mutations and avoids the light chain pairing issue, as exchange of half-Abs can be performed without perturbing the correct heavy chain-light chain interaction. Thus, cFAE can also be used in the present invention in addition to mutations set out herein.

Thus, in one aspect the invention relates to isolated Fc variants, for example canine, equine or feline variants, with one or more modifications in the Fc domain compared to the native wild type sequence wherein the modification is an amino acid substitution in the lower hinge, proline region and/or SHED region wherein the lower hinge comprises residues 119-125, the proline region comprises residues 211-217 and the SHED region comprises residues 151-156 with reference to SEQ ID NO:11.

Mutated regions and their sequence identifiers are set out in FIGS. 2 and 15.

The below provides aspects of the invention with reference to wild type canine IgG-B Fc as shown in SEQ ID No: 11 which shows the canine IgG-B wild type constant region amino acid sequence.

In one aspect, the invention relates to an isolated canine or caninised IgG-B polypeptide or Fc region thereof comprising a modified Fc region wherein said Fc region comprises or consists of
a) an amino acid substitution selected from a modification at position 119 of SEQ ID NO: 11 to G, an amino acid substitution at position 120 of SEQ ID NO: 11 to S or A and/or an amino acid substitution at position 121 of SEQ ID NO: 11 to A; and/or
b) an amino acid substitution selected from a modification at position 153 of SEQ ID NO: 11 to G, an amino acid substitution at position 154 of SEQ ID NO: 11 to R and/or an amino acid substitution at position 156 of SEQ ID NO: 11 to N and/or
c) an amino acid substitution selected from a modification at position 211 of SEQ ID NO: 11 to H, an amino acid substitution at position 212 of SEQ ID NO: 11 to I, an amino acid substitution at position 213 of SEQ ID NO: 11 to G; an amino acid substitution at position 215 of SEQ ID NO: 11 to G and/or an amino acid substitution at position 217 of SEQ ID NO: 11 to S.

For example, for a), the amino acid substitution is at position 120 of SEQ ID NO: 11 to S or A and at position 121 of SEQ ID NO: 11 to A.

For example, for c), the amino acid substitution is an amino acid substitution at position 211 of SEQ ID NO: 11 to H, an amino acid substitution at position 212 of SEQ ID NO: 11 to I and an amino acid substitution at position 213 of SEQ ID NO: 11 to G so that 3 substitutions are present. In one embodiment, additionally, an amino acid substitution at position 215 of SEQ ID NO: 11 to G and/or an amino acid substitution at position 217 of SEQ ID NO: 11 to S is present. In one embodiment of c), the amino acid substitution is at position 213 of SEQ ID NO: 11 to G.

In one embodiment, the Fc region is not an Fc region with only two substitutions namely at position 120 of SEQ ID NO: 11 to A and an amino acid substitution at position 121 of SEQ ID NO: 11 to A and wherein said Fc region is the Fc region of a canine antibody that binds canine CD20. In one embodiment, the Fc region is not an Fc region that binds CD20.

In one embodiment, the Fc region is not an Fc region with only two substitutions namely at position 120 of SEQ ID NO: 11 to A and an amino acid substitution at position 121 of SEQ ID NO: 11 to A.

The term CD20 refers to the B-lymphocyte antigen CD20. The antibodies and antigen binding portions thereof bind specifically to wild type canine CD20 as defined in SEQ ID No. 26 (nucleotide sequence) and SEQ ID No. 27 (amino acid sequence). Unless otherwise stated, the term CD20 as used herein refers to canine CD20. B-lymphocyte antigen CD20 or CD20 is expressed on the surface of all B-cells beginning at the pro-B phase (CD45R+, CD117+) and progressively increasing in concentration until maturity. In humans and canines, CD20 is encoded by the MS4A1 gene.

Thus, with reference to the wild type residue in canine IgG-B constant region (SEQ ID NO: 11), the following amino acid substitutions at the following positions are within the scope of the invention:
E119G;
M120S or A;
L121A;
D153G;
P154R;
D156N;
N211H;
K212I;
A213G;
P215G and/or
P217S.

Thus, E at position 119 in the wild type IgG-B constant region sequence (with reference to SEQ ID NO. 11) is substituted with G and so forth. Various combinations of these substitutions listed above are within the scope of the invention. Thus, the mutants may have 1-11 substitutions as listed above, e.g. 1, 2, 3, 4, 5 or 6 substitutions.

Thus, in one aspect, the invention relates to an isolated canine or caninised IgG-B polypeptide or Fc region thereof comprising a modified Fc region wherein said Fc region comprises
a) an amino acid substitution selected from a modification at position 119 of SEQ ID NO: 11 to G, an amino acid substitution at position 120 of SEQ ID NO: 11 to S or A and/or an amino acid substitution at position 121 of SEQ ID NO: 11 to A; and/or
b) an amino acid substitution at position 156 of SEQ ID NO: 11 to N and/or
c) an amino acid substitution is selected from one of the following modifications
c1) a modification at position 211 of SEQ ID NO: 11 to H in combination with an amino acid substitution at position 212 of SEQ ID NO: 11 to I and an amino acid substitution at position 213 of SEQ ID NO: 11 to G;
c2) an amino acid substitution at position 215 of SEQ ID NO: 11 to G;
c3) an amino acid substitution at position 217 of SEQ ID NO: 11 to S or
c4) a combination of c1) to c3).

In one embodiment, the canine or caninised IgG-B polypeptide or Fc region thereof comprises a modified Fc region wherein said Fc region comprises an amino acid substitution as set out in a) below and optionally an amino acid substitution as set out in b) below and/or c) below wherein
a) the amino acid substitution is selected from a modification at position 119 of SEQ ID NO: 11 to G, an amino acid substitution at position 120 of SEQ ID NO: 11 to S or A and/or an amino acid substitution at position 121 of SEQ ID NO: 11 to A;
b) the amino acid substitution is selected from a modification at position 153 of SEQ ID NO: 11 to G, an amino acid substitution at position 154 of SEQ ID NO: 11 to R and/or an amino acid substitution at position 156 of SEQ ID NO: 11 to N and/or
c) the amino acid substitution is selected from a modification at position 211 of SEQ ID NO: 11 to H, an amino acid substitution at position 212 of SEQ ID NO: 11 to I, an amino acid substitution at position 213 of SEQ ID NO: 11 to G, an amino acid substitution at position 215 of SEQ ID NO: 11 to G and/or an amino acid substitution at position 217 of SEQ ID NO: 11 to S.

In one embodiment, the canine or caninised IgG-B polypeptide or Fc region thereof comprises
a) an amino acid substitution at position 120 of SEQ ID NO: 11 to S and
b) an amino acid substitution at position 211 of SEQ ID NO: 11 to H, an amino acid substitution at position 212 of SEQ ID NO: 11 to I and an amino acid substitution at position 213 of SEQ ID NO: 11 to G.

In one embodiment, the canine or caninised IgG-B polypeptide or Fc region thereof said comprises
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G.

In one embodiment, the canine or caninised IgG-B polypeptide or Fc region comprises
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 217 of SEQ ID NO: 11 to S.

In one embodiment, the canine or caninised IgG-B polypeptide or Fc region thereof comprises
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 215 of SEQ ID NO: 11 to G.

In one embodiment, the canine or caninised IgG-B polypeptide or Fc region thereof comprises an amino acid substitution at position 120 of SEQ ID NO: 11 to A and at position 121 to A, the Fc region is not an Fc region with only two substitutions namely at position 120 of SEQ ID NO: 11 to A and an amino acid substitution at position 121 of SEQ ID NO: 11 to A and wherein said Fc region is the Fc region of a canine antibody that binds canine CD20.

In one embodiment, the canine or caninised IgG-B polypeptide or Fc region thereof comprises
a) an amino acid substitution at position 120 of SEQ ID NO: 11 to A and at position 121 to A and
b) an amino acid substitution at position 217 of SEQ ID NO: 11 to S.

In one embodiment, the canine or caninised IgG-B polypeptide or Fc region thereof comprises
a) an amino acid substitution at position 120 of SEQ ID NO: 11 to A and at position 121 to A and
b) an amino acid substitution at position 215 of SEQ ID NO: 11 to G.

In one embodiment, the canine or caninised IgG-B polypeptide or Fc region thereof comprises
a) an amino acid substitution at position 119 of SEQ ID NO: 11 to G and
b) an amino acid substitution at position 156 of SEQ ID NO: 11 to N.

In one embodiment, the canine or caninised IgG-B polypeptide or Fc region comprises
a) an amino acid substitution at position 120 of SEQ ID NO: 11 to A and at position 121 to A;
b) an amino acid substitution at position 156 of SEQ ID NO: 11 to N and
c) an amino acid substitution at position 217 of SEQ ID NO: 11 to S

In one embodiment, the Fc region includes the modifications as set out above but does not have any further modifications, e.g any other amino acid substitutions or substitutions at other positions in the lower hinge, shed area or proline sandwich.

In one embodiment, the polypeptide or Fc domain comprises or consists of SEQ ID Nos: 12, 13, 14, 15, 16, 17, 18, 19 or 20 or a sequence with at least 90% sequence identity thereto wherein the sequence is not a wild type sequence.

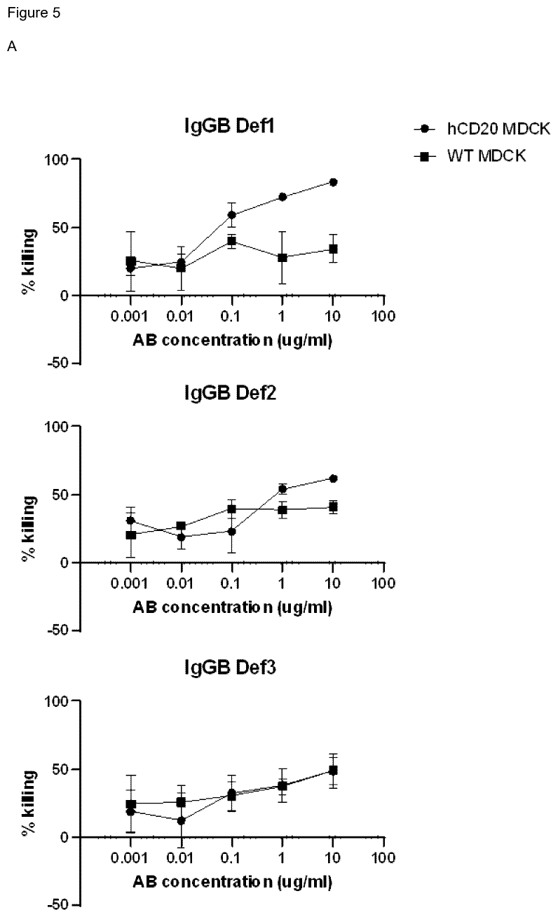

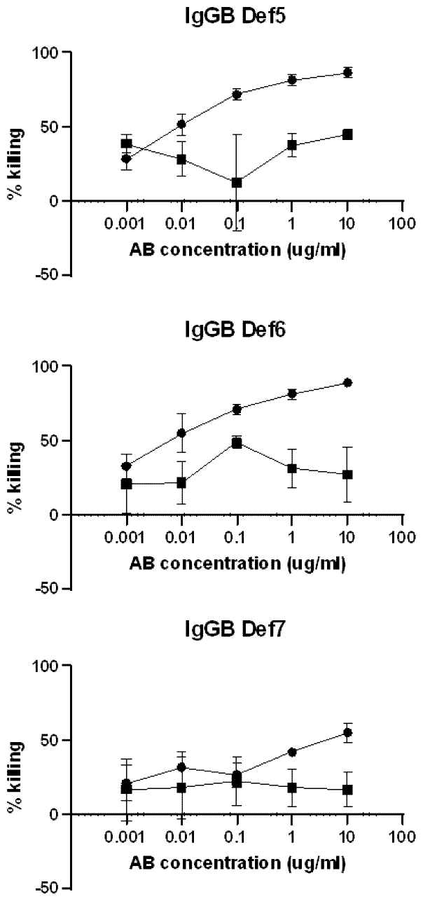

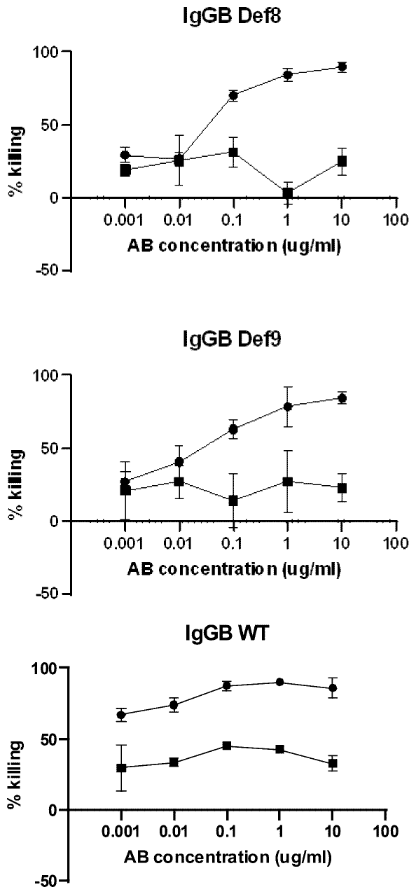

Thus, the invention also relates to an Fc variant defined herein as a canine IgG-B-def mutant 1, 2, 3, 4, 5, 6, 7, 8 or 9 having the modifications as shown in FIG. 2A and as shown in the sequences provided herein, e.g. SEQ ID NOs: 12, 13, 14, 15, 16, 17, 18, 19 and 20. In one embodiment, the polypeptide comprises or consists of SEQ ID Nos: 12, 13, 14, 15, 16, 17, 18, 19 and 20 or a sequence with at least 90% sequence identity thereto wherein the sequence is not a wild type sequence as shown in SEQ ID NO:11. In one embodiment, the canine Fc variant defined is canine IgG-B-def mutant 2, 3 or 7. IgG-B-def mutants 2, 3 or 7 completely inactivate effector function, especially for ADCC activity, shown in FIG. 5.

In one embodiment, the polypeptide comprises or consists of SEQ ID NOs: 2, 3, 4, 5, 6, 7, 8, 9 or 10 or a sequence with at least 90% sequence identity thereto wherein the sequence is not a wild type sequence as shown in SEQ ID No.1 or 11.

The embodiments above relate to canine Fc domains that are modified. Within the scope of the invention are also feline and equine modified Fc domains. A skilled person would be able to determine the equivalent positions for modification in feline and equine Fc domains based on alignment of canine, feline and equine Fc domains.

Thus, the invention also relates to modified feline Fc regions. These have modifications in the lower hinge and/or proline sandwich regions. The below provides aspects of the invention with reference to wild type feline IgG1 Fc as shown in SEQ ID No: 31 which shows the feline IgG1 wild type constant region amino acid sequence. The lower hinge region is at residues 119-125, the proline sandwich at residues 211-217 and the SHED region at residues 151-156.

Thus, with reference to the wild type residue in feline IgG1 constant region (SEQ ID NO: 31), the following amino acid substitutions at the following positions are within the scope of the invention:
M120A;
M120I;
I121A;
I121P;
G123A;
P215G;
P217S and/or
P217A.

Advantageously, the substitution of P217A can be combined with other substitutions in the lower hinge as shown herein.

Thus, in another aspect, the invention relates to a feline or felinised IgG1 polypeptide or Fc region thereof comprising a modified Fc region wherein said Fc region comprises
a) an amino acid substitution at position 120 of SEQ ID NO: 31 to A and an amino acid substitution at position 121 of SEQ ID NO: 31 to A or an amino acid substitution at position 120 of SEQ ID NO: 31 to I and an amino acid substitution at position 121 of SEQ ID NO: 31 to P and/or
b) one or more of the following modifications: an amino acid substitution at position 217 of SEQ ID NO: 31 to S or A, an amino acid substitution at position 215 of SEQ ID NO: 31 to G, and/or an amino acid substitution at position 123 of SEQ ID NO: 31 to A.

In one embodiment, the feline or felinised IgG1 polypeptide or Fc region thereof comprises a modified Fc region wherein said Fc region comprises
a) an amino acid substitution at position 120 of SEQ ID NO: 31 to A, an amino acid substitution at position 121 of SEQ ID NO: 31 to A and further comprising either an amino acid substitution at position 217 of SEQ ID NO: 31 to S or an amino acid substitution at position 215 of SEQ ID NO: 31 to G or
b) an amino acid substitution at position 120 of SEQ ID NO: 31 to I, an amino acid substitution at position 121 of SEQ ID NO: 31 to P and further comprising either an amino acid substitution at position 217 of SEQ ID NO: 31 to A or an amino acid substitution at position 123 of SEQ ID NO: 31 to A.

In one embodiment, the feline or felinised IgG1 polypeptide or Fc region thereof comprises a modified Fc region wherein said Fc region comprises an amino acid substitution at position 120 of SEQ ID NO: 31 to A, an amino acid substitution at position 121 of SEQ ID NO: 31 to A and an amino acid substitution at position 217 of SEQ ID NO: 31 to S.

In one embodiment, the feline or felinised IgG1 polypeptide or Fc region thereof comprises an amino acid substitution at position 120 of SEQ ID NO: 31 to A, an amino acid substitution at position 121 of SEQ ID NO: 31 to A and an amino acid substitution at position 215 of SEQ ID NO: 31 to G.

In one embodiment, the feline or felinised IgG1 polypeptide or Fc region thereof comprises an amino acid substitution at position 120 of SEQ ID NO: 31 to I, an amino acid substitution at position 121 of SEQ ID NO: 31 to P and an amino acid substitution at position 123 of SEQ ID NO: 31 to A and optionally an amino acid substitution at position 217 of SEQ ID NO: 31 to A.

In one embodiment, the feline or felinised IgG1 polypeptide or Fc region thereof comprises an amino acid substitution at position 120 of SEQ ID NO: 31 to I, an amino acid substitution at position 121 of SEQ ID NO: 31 to P and an amino acid substitution at position 217 of SEQ ID NO: 31 to A.

In one embodiment, the polypeptide has reduced Fc-mediated effector function when compared to the same polypeptide comprising a wild-type IgG1 Fc domain.

In one embodiment, the polypeptide comprises or consists of SEQ ID NOs: 38, 39, 40 or 41 or a sequence with at least 90% sequence identity thereto wherein the sequence is not a wild type IgG sequence, e.g. as shown in SEQ ID No.31.

In one embodiment, the feline Fc variant defined is feline IgG1-def mutant 2, or 3. IgG-1def mutants 2 and 3 completely inactivate effector function.

In one embodiment, the Fc region includes the modifications as set out above but does not have any further modifications, e.g any other amino acid substitutions or substitutions at other positions in the lower hinge, shed area or proline sandwich.

The embodiments relate to isolated polypeptides, e.g. a canine IgG-B polypeptide or feline IgG1 polypeptide respectively, that comprise a modified Fc domain and to isolated Fc domains that comprise one or more modification as set out above. A skilled person would therefore understand that the modified Fc domain as described herein can be used in any antibody, e.g. canine or feline antibody respectively. As such, the Fc domain may be linked to a variable domain that provides specific antigen binding. Suitable examples are given in the example section.

In one embodiment, the polypeptide or Fc domain may have one or more additional modification. In one embodiment, the polypeptide or Fc domain described herein does not have any additional modifications, e.g. amino acid substitutions, in the Fc domain other than those identified herein.

In one embodiment, the canine or caninised IgG-B polypeptide or Fc region thereof has reduced Fc-mediated effector function when compared to the same polypeptide comprising a wild-type IgG-B Fc domain. In one embodiment, the feline or felinised IgG1 polypeptide or Fc region thereof has reduced Fc-mediated effector function when compared to the same polypeptide comprising a wild-type IgG-1 Fc domain.

Reduction as used herein can be at least 10%, 20%, 30%, 40%, 50% or greater.

In one embodiment, the effector function is antibody-dependent cell-mediated cytotoxicity (ADCC) or antibody dependent cell-mediated phagocytosis (ADCP).

By “ADCC” or “antibody dependent cell-mediated cytotoxicity” as used herein is meant the cell-mediated reaction wherein nonspecific cytotoxic cells that express FcvRs recognize bound antibody on a target cell and subsequently cause lysis of the target cell. ADCC is correlated with binding to FcYRIIIa; increased binding to FcYRII Ia leads to an increase in ADCC activity.

By “ADCP” or antibody dependent cell-mediated phagocytosis as used herein is meant the cell-mediated reaction wherein nonspecific cytotoxic cells that express FcvRs recognize bound antibody on a target cell and subsequently cause phagocytosis of the target cell.

In one embodiment, there is reduction of ADCC activity. In one embodiment, there is abolishment of ADCC activity.

In one embodiment, the Fc mutant is fully CDC and ADCC deficient.

Advantageously, the Fc variants described herein retain good biophysical properties, as shown in FIGS. 7, 8, 18 and 19.

Various assays to measure the activities of the Fc variants are shown in the examples.

In one embodiment, the effector function is complement-dependent cytotoxicity (CDC). The term “CDC” as used herein refers to complement dependent cytotoxicity, i.e., a biochemical event of targeted cell destruction mediated by the complement system.

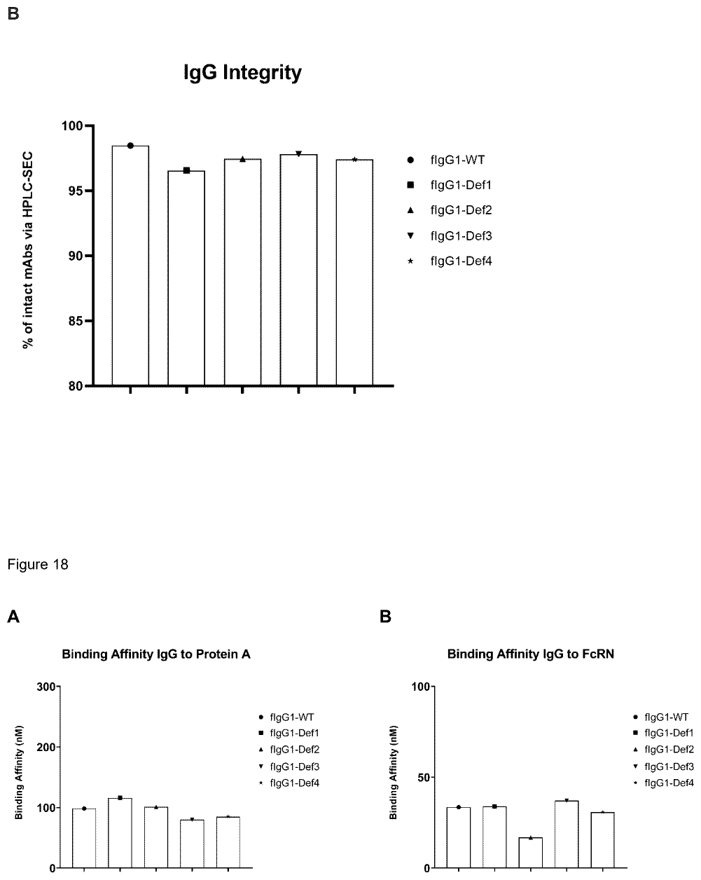

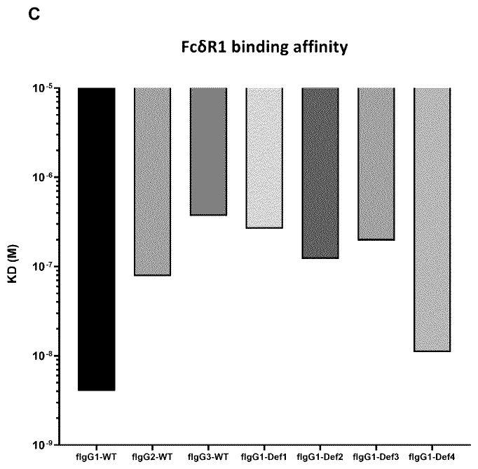

FIGS. 7 and 18 show protein A binding. The modifications in the canine IgG-B do not alter protein A and FcRn binding. The modifications in the feline IgG1 do not alter protein A and FcRn binding.

In one embodiment, the canine or caninised IgG-B or feline or felinised IgG1 polypeptide or Fc region thereof has lower affinity for an Fc gamma receptor (FcγR) when compared to the same polypeptide comprising a wild-type IgG-B Fc domain. Affinity may be reduced by at least 10%, 20%, 30%, 40%, 50% or more.

In one embodiment, the FcγR is selected from the group consisting of FcγRI, FcγRII, and FcγRIII.

In one embodiment, the canine or caninised IgG-B polypeptide or Fc region thereof retains FcRn binding, but reduces or abolishes effector functions. In one embodiment, the feline or felinised IgG1 polypeptide or Fc region thereof retains FcRn binding, but reduces or abolishes effector functions.

The polypeptide may be part of a full-length antibody or antibody fragment. In one embodiment, the antibody is a multispecific antibody or fragment thereof. A multispecific protein, e.g. a multispecific antibody binds to at least two different targets, i.e. is at least bispecific. Thus, in one embodiment, the antibody is a bispecific antibody or fragment thereof. In other embodiment, the mutispecific antibody or fragment thereof binds to three, four or more targets.

Bispecific antibodies of the invention based on the IgG format, comprising of two heavy and two light chains can be produced by a variety of methods known in the art. For instance, bispecific antibodies may be produced by fusing two antibody-secreting cell lines to create a new cell line or by expressing two antibodies in a single cell using recombinant DNA technology. These approaches yield multiple antibody species as the respective heavy chains from each antibody may form monospecific dimers (also called homodimers), which contain two identical paired heavy chains with the same specificity, and bispecific dimers (also called heterodimers) which contain two different paired heavy chains with different specificity. In addition, light chains and heavy chains from each antibody may randomly pair to form inappropriate, non-functional combinations. This problem, known as heavy and light chain miss-pairings, can be solved by choosing antibodies that share a common light chain for expression as bispecifics. Methods to address the light chain-heavy chain mispairing problem include the generation of bispecific antibodies using a single light chain. This requires heavy-light chain engineering or novel antibody libraries that utilize a single light chain that limits the diversity. In addition, antibodies with a common light chain have been identified from transgenic mice with a single light chain. Another approach is to swap the CH1 domain of one heavy chain with CL domain of its cognate light chain (Crossmab technology). Also covered are scFv formats.

In one embodiment, in particular for the treatment of cancer, the protein may target CD3 and is provided in the format of a bispecific T-cell engager (BiTE).

In one embodiment, the protein is multiparatopic, i.e. binds to more than one epitope on the same target.

A bispecific antibody has specificity for no more than two epitopes. A bispecific antibody is characterized by a first immunoglobulin variable domain sequence which has binding specificity for a first epitope and a second immunoglobulin variable domain sequence that has binding specificity for a second epitope. In one embodiment, the first and second epitopes are on the same antigen, e.g., the same protein (or subunit of a multimeric protein). In one embodiment, the first and second epitopes overlap. In an embodiment, the first and second epitopes do not overlap. In one embodiment, the first and second epitopes are on different antigens, e.g., the different proteins (or different subunits of a multimeric protein). In another embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In a further embodiment, a bispecific antibody molecule comprises an antibody having binding specificity for a first epitope and an antibody having binding specificity for a second epitope. In one embodiment, a bispecific antibody molecule comprises an antibody, or fragment thereof, having binding specificity for a first epitope and a half antibody, or fragment thereof, having binding specificity for a second epitope. In one embodiment, a bispecific antibody or fragment thereof comprises a Fab having binding specificity for a first epitope and a Fab having binding specificity for a second epitope.

Also within the scope of the invention are scFv formats.

In another embodiment, the polypeptide, Fc polypeptide or antibody or fragment thereof comprises a further moiety.

For example, the protein is an Fc receptor fusion protein. By “Fc fusion protein” or “immunoadhesin” herein is meant a protein comprising an Fc region, generally linked (optionally through a linker moiety, as described herein) to a different protein, such as a binding moiety to a target protein, as described herein.

For example, the Fc polypeptide as described herein may be combined with a binding domain capable of specifically binding to a target molecule. Thus, in aspect, the invention relates to fusion proteins comprising a Fc region as described herein. In the fusion protein, one or more polypeptide is operably linked to an Fc region of the invention. For example, the Fc domain may be linked to a Fab binding domain. The binding domain may comprise more than one polypeptide chain in association e.g. covalent or otherwise (e.g. hydrophobic interaction, ionic interaction, or linked via sulphide bridges).

Modifications described herein can also be combined with any other Fc modification e.g. with modified effector function.

Generally, the one or more polypeptide operably linked to an Fc region of the invention may be any protein or small molecule, for example a binding domain derived from any molecule with specificity for another molecule and capable of binding said molecule. The binding domain will have an ability to interact with a target molecule which will preferably be another polypeptide, but may be any target (e.g. carbohydrate, lipid (such as phospholipid) or nucleic acid). Preferably, the interaction will be specific. Typically, the target will be an antigen present on a cell, or a receptor with a soluble ligand. This may be selected as being a therapeutic target, whereby it is desired to bind it with a molecule having the properties discussed above. The target may be present on or in a target cell, for example a target cell which it is desired to lyse, or in which it is desired to induce apoptosis. Protein fusion partners may thus include, but are not limited to, the variable region of any antibody, the target-binding region of a receptor, an adhesion molecule, a ligand, an enzyme, a cytokine, an antigen, a chemokine, or any other protein or protein domain. Small molecule fusion partners may include any therapeutic agent that directs the Fc fusion to a therapeutic target. Such targets may be any molecule, preferably an extracellular receptor, which is implicated in disease.

In another embodiment, the antibody of the invention comprises a further moiety. For example, the moiety is a half life extending moiety, e.g. a canine or caninized serum albumin or a variant thereof or a feline or felinised serum albumin or a variant thereof. The antibody may also be modified to increase half-life, for example by a chemical modification, especially by PEGylation, or by incorporation in a liposome.

Half-life may be increased by at least 1.5 times, preferably at least 2 times, such as at least 5 times, for example at least 10 times or more than 20 times, greater than the half-life of the corresponding antibody without the half life extension. For example, increased half-life may be more than 1 hours, preferably more than 2 hours, more preferably more than 6 hours, such as more than 12 hours, or even more than 24, 48 or 72 hours, compared to the corresponding antibody without the half life extending moiety.

In another embodiment, the moiety is a therapeutic moiety, such as a drug, an enzyme or a toxin. In one embodiment, the therapeutic moiety is a toxin, for example a cytotoxic radionuclide, chemical toxin or protein toxin. Thus, the invention also encompasses an immunoconjugate, e.g. an antibody conjugated (joined) to a second molecule, usually a toxin, radioisotope or label. These conjugates are used in immunotherapy and to develop monoclonal antibody therapy as a targeted form of chemotherapy when they are often known as antibody-drug conjugates (ADCs).

The toxic payload may be selected from small molecules (e.g. maytansanoid, auristatin), a protein toxin (e.g. Pseudomonas exotoxin, diphtheria toxin), a cytolytic immunomodulatory protein (e.g. Fas ligand) to kill targeted cells, a biologically active peptide (e.g. GLP-1) to extend the pharmacological half-life of the natural peptide, an enzymes (e.g. urease) to modify the biochemistry of the targeted microenvironment or radionuclides (e.g. 90Y, 111In) for either killing or imaging of tumor cells.

In another embodiment, the antibody is labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

The moiety can be linked to the polypeptide, e.g. antibody, using linkers known in the art, e.g. via a chemical or peptide linker. The linkage can be covalent or non-covalent. An exemplary covalent linkage is via a peptide bond. In some embodiments, the linker is a polypeptide linker (L). Suitable linkers include for example a linker with GS residues such as (Gly4Ser)n where n can be 1 to 20. Other linkage/conjugation techniques include cysteine conjugation, e.g. for cysteine-based site-specific antibody conjugation to a toxic payload.

In another aspect, the invention relates to a nucleic acid molecule encoding the polypeptide of the invention. The nucleic acid is preferably an isolated nucleic acid.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature.

Furthermore, we provide an isolated nucleic acid construct comprising at least one nucleic acid as defined above. The construct may be in the form of a plasmid, vector, transcription or expression cassette. Thus, the invention also relates to a plasmid, vector, transcription or expression cassette comprising a nucleic acid of the invention. Expression vectors of use in the invention may be constructed from a starting vector such as a commercially available vector. After the vector has been constructed and a nucleic acid molecule encoding a heavy chain, or a light chain and a heavy chain sequence has been inserted into the proper site of the vector, the completed vector may be inserted into a suitable host cell for amplification and/or polypeptide expression.

The term “vector” means a construct, which is capable of delivering, and in some aspects expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The invention also relates to an isolated recombinant host cell comprising one or more nucleic acid molecule plasmid, vector, transcription or expression cassette as described above. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

The host cell may be eukaryotic or prokaryotic, for example a bacterial, viral, plant, fungal, mammalian or other suitable host cell. In one embodiment, the cell is an E. coli cell. In another embodiment, the cell is a yeast cell. In another embodiment, the cell is a Chinese Hamster Ovary (CHO) cell, HeLa cell or other cell that would be apparent to the skilled person. Mammalian cell lines available as hosts for expression are well known in the art and include, but are not limited to, immortalized cell lines available from the American Type Culture Collection (ATCC) and any cell lines used in an expression system known in the art can be used to make the recombinant polypeptides of the invention.

In general, host cells are transformed with a recombinant expression vector that comprises DNA encoding a desired bispecific antibody. Among the host cells that may be employed are prokaryotes, yeast or higher eukaryotic cells. Prokaryotes include gram negative or gram-positive organisms, for example E. coli or bacilli. Higher eukaryotic cells include insect cells and established cell lines of mammalian origin. Examples of suitable mammalian host cell lines include the COS-7 cells, L cells, Cl27 cells, 3T3 cells, Chinese hamster ovary (CHO) cells, or their derivatives and related cell lines which grow in serum free media, HeLa cells, BHK cell lines, the CVIIEBNA cell line, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays.

Other suitable host cells include insect cells, using expression systems such as baculovirus in insect cells, plant cells, transgenic plants and transgenic animals, and by viral and nucleic acid vectors.

Alternatively, it is possible to produce the polypeptide in lower eukaryotes such as fungal cell lines and yeast or in prokaryotes such as bacteria. Suitable yeasts include S. cerevisiae, S. pombe, Kluyveromyces strains, Pichia pastoris, Candida, or any yeast strain capable of expressing heterologous polypeptides. Suitable bacterial strains include E. coli, B. subtilis, S. typhimurium, or any bacterial strain capable of expressing heterologous polypeptides. If the bispecific antibody is made in yeast or bacteria, it may be desirable to modify the product produced therein, for example by phosphorylation or glycosylation of the appropriate sites, in order to obtain a functional product. Such covalent attachments can be accomplished using known chemical or enzymatic methods.

A host cell, when cultured under appropriate conditions, can be used to express a bispecific antibody that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

A skilled person will know that there are different ways to identify, obtain and optimise the polypeptides as described herein, including in vitro and in vivo expression libraries. Optimisation techniques known in the art, such as display (e.g., ribosome and/or phage display) and/or mutagenesis (e.g., error-prone mutagenesis) can be used.

In such a method, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition, Brian K. Kay, Jill Winter, John McCafferty, 1996).

Libraries, for example phage libraries, are generated by isolating a cell or tissue expressing an antibody or heavy chain, cloning the sequence encoding the genes from mRNA derived from the isolated cell or tissue and displaying the encoded protein using a library. The heavy chain can be expressed in mammalian, bacterial, yeast or other expression systems.

Therefore, in another aspect, the invention also relates to an expression library comprising a plurality of polypeptides, Fc domains or antibodies as described herein. Phage display library screening is advantageous over some other screening methods due to the vast number of different polypeptides (typically exceeding 10⁹) that can be contained in a single phage display library. This allows for the screening of a highly diverse library in a single screening step. In general, the libraries include repertoires of V genes (e.g., harvested from populations of lymphocytes or assembled in vitro) which are cloned for display of associated heavy and light chain variable domains on the surface of filamentous bacteriophage. Phages are selected by binding to an antigen. Soluble antibodies are expressed from phage infected bacteria and the antibody can be improved, such as, by mutagenesis. Methods of producing antibodies by making, screening and evolving antibodies and antibody libraries are established.

In a further aspect, the invention relates to a method for making a canine variant Fc domain as described herein comprising introducing an amino acid substitution in the IgG-B Fc domain wherein said amino acid substitution is selected from
a) an amino acid substitution at position 119 of SEQ ID NO: 11 to G, an amino acid substitution at position 120 of SEQ ID NO: 11 to S or A and/or an amino acid substitution at position 121 of SEQ ID NO: 11 to A; and/or
b) an amino acid substitution at position 156 of SEQ ID NO: 11 to N and/or
c) an amino acid substitution is selected from one of the following modifications
c1) a modification at position 211 of SEQ ID NO: 11 to H in combination with an amino acid substitution at position 212 of SEQ ID NO: 11 to I and an amino acid substitution at position 213 of SEQ ID NO: 11 to G;
c2) an amino acid substitution at position 215 of SEQ ID NO: 11 to G;
c3) an amino acid substitution at position 217 of SEQ ID NO: 11 to S or
c4) a combination of c10 to c30.

In one aspect, the amino acid substitution is as set out in a) below and optionally an amino acid substitution as set out in b) below and/or c) below wherein
a) the amino acid substitution is selected from a modification at position 119 of SEQ ID NO: 11 to G, an amino acid substitution at position 120 of SEQ ID NO: 11 to S or A and/or an amino acid substitution at position 121 of SEQ ID NO: 11 to A;
b) the amino acid substitution is selected from a modification at position 153 of SEQ ID NO: 11 to G, an amino acid substitution at position 154 of SEQ ID NO: 11 to R and/or an amino acid substitution at position 156 of SEQ ID NO: 11 to N and/or
c) the amino acid substitution is selected from a modification at position 211 of SEQ ID NO: 11 to H, an amino acid substitution at position 212 of SEQ ID NO: 11 to I, an amino acid substitution at position 213 of SEQ ID NO: 11 to G, an amino acid substitution at position 215 of SEQ ID NO: 11 to G and/or an amino acid substitution at position 217 of SEQ ID NO: 11 to S.

In one aspect, the amino acid substitution is
a) an amino acid substitution at position 120 of SEQ ID NO: 11 to S and
b) an amino acid substitution at position 211 of SEQ ID NO: 11 to H, an amino acid substitution at position 212 of SEQ ID NO: 11 to I and an amino acid substitution at position 213 of SEQ ID NO: 11 to G.

In one aspect, the amino acid substitution is
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G.

In one aspect, the amino acid substitution is
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 217 of SEQ ID NO: 11 to S.

In one aspect, the amino acid substitution is
a) an amino acid substitution at position 120 of SEQ ID NO: 11 to S;
b) an amino acid substitution at position 153 of SEQ ID NO: 11 to G and an amino acid substitution at position 154 of SEQ ID NO: 11 to R and
c) an amino acid substitution at position 211 of SEQ ID NO: 11 to H, an amino acid substitution at position 212 of SEQ ID NO: 11 to I and an amino acid substitution at position 213 of SEQ ID NO: 11 to G and an amino acid substitution at position 215 of SEQ ID NO: 11 to G.

In one aspect, the amino acid substitution is an amino acid substitution at position 120 of SEQ ID NO: 11 to A and at position 121 to A.

In one aspect, the amino acid substitution is
a) an amino acid substitution at position 120 of SEQ ID NO: 11 to A and at position 121 to A and
b) an amino acid substitution at position 217 of SEQ ID NO: 11 to S.

In one aspect, the amino acid substitution is
a) an amino acid substitution at position 120 of SEQ ID NO: 11 to A and at position 121 to A and
b) an amino acid substitution at position 215 of SEQ ID NO: 11 to G.

In one aspect, the amino acid substitution is
a) an amino acid substitution at position 119 of SEQ ID NO: 11 to G and
b) an amino acid substitution at position 156 of SEQ ID NO: 11 to N.

In one aspect, the amino acid substitution is
a) an amino acid substitution at position 120 of SEQ ID NO: 11 to A and at position 121 to A;
b) an amino acid substitution at position 156 of SEQ ID NO: 11 to N and
c) an amino acid substitution at position 217 of SEQ ID NO: 11 to S.

In a further aspect, the invention relates to a method for making a feline variant Fc domain as described herein comprising introducing an amino acid substitution in the IgG1 Fc domain wherein said amino acid substitution is selected from
a) an amino acid substitution at position 120 of SEQ ID NO: 31 to A and an amino acid substitution at position 121 of SEQ ID NO: 31 to A or an amino acid substitution at position 120 of SEQ ID NO: 31 to I and an amino acid substitution at position 121 of SEQ ID NO: 31 to P and/or
b) one or more of the following modifications: an amino acid substitution at position 217 of SEQ ID NO: 31 to S or A, an amino acid substitution at position 215 of SEQ ID NO: 31 to G, and/or an amino acid substitution at position 123 of SEQ ID NO: 31 to A.

In one embodiment, the substitution is selected from
a) an amino acid substitution at position 120 of SEQ ID NO: 31 to A, an amino acid substitution at position 121 of SEQ ID NO: 31 to A and further comprising either an amino acid substitution at position 217 of SEQ ID NO: 31 to S or an amino acid substitution at position 215 of SEQ ID NO: 31 to G or
b) an amino acid substitution at position 120 of SEQ ID NO: 31 to I, an amino acid substitution at position 121 of SEQ ID NO: 31 to P and further comprising either an amino acid substitution at position 217 of SEQ ID NO: 31 to A or an amino acid substitution at position 123 of SEQ ID NO: 31 to A.

In one embodiment, the feline or felinised IgG-1 polypeptide or Fc region thereof comprises a modified Fc region wherein said Fc region comprises an amino acid substitution at position 120 of SEQ ID NO: 31 to A, an amino acid substitution at position 121 of SEQ ID NO: 31 to A and an amino acid substitution at position 217 of SEQ ID NO: 31 to S.

In one embodiment, the substitution is selected from an amino acid substitution at position 120 of SEQ ID NO: 31 to A, an amino acid substitution at position 121 of SEQ ID NO: 31 to A and an amino acid substitution at position 215 of SEQ ID NO: 31 to G.

In one embodiment, the substitution is selected from an amino acid substitution at position 120 of SEQ ID NO: 31 to I, an amino acid substitution at position 121 of SEQ ID NO: 31 to P and an amino acid substitution at position 123 of SEQ ID NO: 31 to A and optionally an amino acid substitution at position 217 of SEQ ID NO: 31 to A.

In one embodiment, the substitution is selected from an amino acid substitution at position 120 of SEQ ID NO: 31 to I, an amino acid substitution at position 121 of SEQ ID NO: 31 to P and an amino acid substitution at position 217 of SEQ ID NO: 31 to A.

The parent polypeptide may be a wild type polypeptide, e.g. Fc domain or polypeptide comprising an Fc domain, or one that has modifications compared to the wild type sequence.

In another aspect, there is provided a pharmaceutical composition comprising a polypeptide or Fc domain of the invention, e.g. a canine, cananised, feline or felinised Fc domain or polypeptide comprising an Fc domain as described herein, and optionally a pharmaceutically acceptable carrier. A polypeptide or pharmaceutical composition described herein can be administered by any convenient route, including but not limited to oral, topical, parenteral, sublingual, rectal, vaginal, ocular, intranasal, pulmonary, intradermal, intravitrial, intratumoural, intramuscular, intraperitoneal, intravenous, subcutaneous, intracerebral, transdermal, transmucosal, by inhalation, or topical, particularly to the ears, nose, eyes, or skin or by inhalation. In another embodiment, delivery is of the nucleic acid encoding the drug, e.g. a nucleic acid encoding the molecule of the invention is delivered.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the polypeptide of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition can be in the form of a liquid, e.g., a solution, syrup, solution, emulsion or suspension. The liquid can be useful for oral administration or for delivery by injection, infusion (e.g., IV infusion) or sub-cutaneous.

When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the polypeptide, Fc domain or pharmaceutical composition described herein that is effective/active in the treatment of a particular disease or condition will depend on the nature of the disease or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disease, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of a polypeptide, Fc domain of the present invention by weight of the composition. When intended for oral administration, this amount can be varied to range from about 0.1% to about 80% by weight of the composition. Preferred oral compositions can comprise from about 4% to about 50% of the polypeptide of the present invention by weight of the composition.

Compositions can be prepared so that a parenteral dosage unit contains from about 0.01% to about 2% by weight of the polypeptide or Fc domain of the present invention.

For administration by injection, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks.

The invention also relates to therapeutic treatment applications of the polypeptide, Fc domain, e.g. antibody or fragment thereof, or pharmaceutical composition as described herein.

As used herein, “treat”, “treating” or “treatment” means inhibiting or relieving a disease or disease. For example, treatment can include a postponement of development of the symptoms associated with a disease or disease, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., canine patients, being treated. Many medical treatments are effective for some, but not all, patients that undergo the treatment.

The term “subject” or “patient” refers to an animal which is the object of treatment, observation, or experiment, suitably a companion animal, specifically a canine.

The invention also relates to a polypeptide, Fc domain or pharmaceutical composition described herein for use in the treatment or prevention of a disease.

In another aspect, the invention relates to the use of a polypeptide, Fc domain or pharmaceutical composition described herein in the treatment or prevention of a disease. In another aspect, the disclosure relates to the use of a polypeptide, Fc domain or pharmaceutical composition described herein in the manufacture of a medicament for the treatment or prevention of a disease as listed herein. The invention further relates to a method of treating a disease in a subject comprising an effective amount of the polypeptide, Fc domain or pharmaceutical composition as described herein to said subject.

For example, the disease is selected from an inflammatory or autoimmune disease.

The disease may be an inflammatory skin diseases, including atopic dermatitis, allergic dermatitis, pruritus, psoriasis, scleroderma, or eczema; responses associated with inflammatory bowel disease (such as Crohn's disease and ulcerative colitis); ischemic reperfusion; adult respiratory distress syndrome; asthma; meningitis; encephalitis; uveitis; autoimmune diseases such as rheumatoid arthritis, Sjorgen's syndrome, vasculitis; diseases involving leukocyte diapedesis; central nervous system (CNS) inflammatory disorder, multiple organ injury syndrome secondary to septicaemia or trauma, bacterial pneumonia, antigen-antibody complex mediated diseases; inflammations of the lung, including pleurisy, alveolitis, vasculitis, pneumonia, chronic bronchitis, bronchiectasis, and cystic fibrosis.

In one embodiment, the antibody or fragment, binding molecule, pharmaceutical composition is administered together with one or more therapeutic agent.

For example, said one or more therapeutic agent is selected from rapamycin (sirolimus), tacrolimus, cyclosporine (e.g. Atopica®), corticosteroids (e.g. methylprednisolone), methotrexate, mycophenolate mofetil, anti-CD28 antibodies, anti-IL12/IL-23 antibodies, anti-CD20 antibodies, anti-CD30 antibodies, CTLA4-Fc molecules, CCR5 receptor antagonists, anti-CD40L antibodies, anti-VI_A4 antibodies, anti-LFA1 antibodies, fludarabine, anti-CD52 antibodies, anti-CD45 antibodies, cyclophosphamide, anti-thymocyte globulins, anti-complement C5 antibodies, anti-a4b7 integrin antibodies, anti-IL6 antibodies, anti-IL6-R antibodies, anti-IL2R antibodies, anti-CD25 antibodies, anti-TNFa/TNFa-Fc molecules, HDAC inhibitors, JAK inhibitors, such as JAK-1 and JAK-3 inhibitors, anti-IL-31 antibodies, SYK inhibitors, anti-IL-4Ra antibodies, anti-IL-13 antibodies, anti-TSLP antibodies, PDE4 inhibitors, Lokietmab (Cytopoint®), and Oclacitinib (Apoquel®).

The polypeptide, Fc domain or pharmaceutical composition described herein may be administered at the same time or at a different time as the other therapy or therapeutic compound or therapy, e.g., simultaneously, separately or sequentially.

The invention also provides an in vitro or in vivo method for repressing effector function comprising contacting a cell or tissue with a polypeptide or Fc region thereof as described herein. The effector function may be antibody-dependent cell-mediated cytotoxicity (ADCC), or antibody dependent cell-mediated phagocytosis (ADCP) or complement-dependent cytotoxicity (CDC).

In another aspect, the invention provides a kit for the treatment or prevention of a disease, diagnosis, prognosis or monitoring disease comprising a polypeptide, Fc domain or pharmaceutical composition of the invention. Such a kit may contain other components, packaging and/or instructions.

The kit may include a labelled polypeptide, Fc domain or pharmaceutical composition as described herein and one or more compounds for detecting the label.

The invention in another aspect provides a polypeptide, Fc domain or pharmaceutical composition as described herein of the invention packaged in lyophilized form or packaged in an aqueous medium.

In another aspect, a polypeptide or Fc domain as described herein is used for non-therapeutic purposes, such as diagnostic tests and assays.

Further aspects and embodiments of the invention will be apparent to those skilled in the art given the present disclosure including the following experimental exemplification.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including any references to gene accession numbers and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further described in the non-limiting examples.

### EXAMPLES

### Methods

### 1. Canine Antibody Constructs

The amino acid sequences for wild type canine IgG A, B (SEQ ID NO:1), C and D are given in FIG. 1A. FIG. 2 is a table showing the amino acid substitutions to generate effector function deficient canine IgG B variants.

For antibody production, DNA constructs were generated to encode chimeric antibodies comprising selected canine and IgG constant regions fused to the variable regions of anti-human CD20 antibodies, Rituximab (see U.S. Pat. No. 5,736,137) or Ofatumumab (sequence available on Drugbank https://go.drugbank.com/drugs/DB06650). Wildtype canine immunoglobulins were assessed by SDS-PAGE (using 4-12% Bis-Tris NuPAGE gels, MES SDS running buffer and SeeBlue Plus 2 standards, all from ThermoFisher) and are shown in FIG. 1B.

The canine IgG-B mutant variants (Def 1 to 9, Seq ID 2 to 10 and 12 to 20) were generated by site directed mutagenesis. Specifically, human Ofatumumab or Rituximab variable region and canine IgG-B mutant variants are PCR amplified using Q5 high fidelity DNA polymerase and assembled into mammalian expression vector PetML5 using NEBuilder HIFI DNA Assembly (New England Biolabs). In the expression vector, the heavy chain and the antibiotic resistant gene expression units are flanked by DNA transposon piggyBac terminal inverted repeats to mediate stable integration into host cells in the presence of piggyBac transposase. The expression vectors encoding the heavy chain and light chain are co-transfected into a suitable mammalian cell line such as CHO cells together with PiggyBac transposase to obtain stable expression. For antibody production, 3×10⁶ selected CHO cells are seeded in 3 ml culture media and incubated at 32° C., 5% CO₂ with shaking at 200 rpm. 4% HyClone Cell Boost 7a supplement+0.4% HyClone Cell Boost 7b supplement+1% glucose is added to the media on days 1, 4, 7 and 10. Culture supernatants are collected on day 12 and the IgG concentration determined using surface plasmon resonance using protein A chip (Biacore 8K, Cytiva Life Sciences).

### 2. Feline Antibody Constructs

Genomic regions encompassing the soluble isoforms of IGHG1 and IGHG2 were validated in house by PacBio sequencing. Bacterial artificial chromosome (BAC) clone FCAB-160A15, sourced from a commercial BAC library available at Texas A&M University, was found to encompass the feline immunoglobulin heavy chain genes. PacBio sequencing of the BAC revealed the gene structures and sequences of the known isoforms IGHG1 and IGHG2. Interestingly, a previously unknown third IgG family member, designated here as IGHG3, with the soluble isoform being composed of exons CH1, H, CH2, and CH3-CHS, was also identified by homology search to feline IGHG1 and IGHG2 gene structures. The IGHG3 gene structure is located between IGHG1 and IGHG2, with IGHG3 exon CH1 being positioned approximately 13 kb downstream of IGHG1 exon M2 and IGHG3 exon CH3-CHS approximately 17 kb upstream of IGHG2 exon CH1. In addition to PacBio sequencing, the genomic region encompassing the exons of the soluble IGHG1, IGHG2 and IGHG3 isoforms have been confirmed by Sanger sequencing using FCAB-160A15 as the template. Oligos used for IGHG1 PCR and sequencing (1895 bp): Forward: TGCAGCCGCAGAAACACAAG (SEQ ID NO. 42); Reverse: ATGGTGACCCCTGAGCCTCG (SEQ ID NO. 43). Oligos used for IGHG2 PCR and sequencing (1895 bp): Forward: TGCAGCCGCAGAAACACGAG (SEQ ID NO. 44); Reverse: ATGGTGACCCCTGAGCCTCA (SEQ ID NO. 45). Oligos used for IGHG3 PCR and sequencing (1869 bp): Forward: CAGCCGCAGAAACATGAGGTC (SEQ ID NO. 46); Reverse: ATGGTGACCCCTGAGCCTTG (SEQ ID NO. 47).

The deduced gene structure of the soluble IGHG3 can be found in FIG. 12. The genomic sequence encompassing the soluble isoform of feline IGHG1, IGHG2 and IGHG3 are provided with SEQ ID NO: 28, 29 and 30, with the coding regions underlined in bold. The amino acid sequences for wild type feline IgG 1, 2 and 3 (SEQ ID NO:31-33) are given in FIG. 13A. Wildtype feline immunoglobulins were assessed by SDS-Page (using 4-12% Bis-Tris NuPAGE gels, MES SDS running buffer and SeeBlue Plus 2 standards, all from Invitrogen) and are shown in FIG. 13B. FIG. 15A is a table showing the amino acid substitutions to generate effector function deficient feline IgG 1 variants.

The feline IgG WT and IgG1 mutant variants (Def 1 to 4, SEQ ID 34 to 37 and 38 to 41) were generated in the same manner as that of the canine variants using the human Ofatumumab variable region.

### 3. Complement Dependent Cytotoxicity (CDC) Activity

Canine lymphoid tumour cell lines such as CLBL1 (University of Veterinary Medicine Vienna) or CLC (Umeki S. et al J. Vet. Med. Sci., 75:467-474 (2013) PubMed=23196801; DOI=10.1292/jvms.12-0448) may be used as target cells. These cells are stably transfected or nucleofected with an expression construct encoding for the human CD20 protein (Seq ID 25) to generate hCD20-expressing cells. The expression constructs were generated using the same method as described above for antibody expression.

Transfected cells were selected for puromycin resistance and hCD20^{high} (top 5%) cells were FACS sorted by staining for hCD20 expression using anti-human CD20 antibody (clone: 2H7, BioLegend).

To assess CDC activity, untransfected (wild type) target lymphoid cells or equivalent hCD20-expressing cells were used in a cell killing assay in which 5000 target cells per well of 96-well plate were incubated with anti-human CD20 canine or feline Fc chimeric antibody as described above and canine complement preserved serum (BioIVT) at a final dilution of 1:12, for 2 hours at 37° C., 5% CO₂. The assay was set up using media (RPMI+1% L-glutamine+20% fetal bovine serum for CLBL-1 cells made using heat inactivated serum so that canine complement preserved serum would be the only source of complement.

Live cells were then quantified using CellTitre-Glo® Luminescent Cell Viability Assay (Promega) following the assay protocol. This assay uses the ATP content of live cells as an indication of cell viability. Luminescence was measured on a CLARIOstar (BMG Labtech). Data was analyzed using MARS software (BMG Labtech) and the number of live cells remaining was used to calculate the percentage of killing in the presence of antibodies using Microsoft Excel. Background signal was obtained from a sample of cells treated with 1% triton (where no cells were left alive) and subtracted from the signal obtained from each test sample. Max signal (0% killing) was obtained from a sample of cells treated identically but where antibodies were omitted. Graphs were plotted in Microsoft Excel or GraphPad Prism.

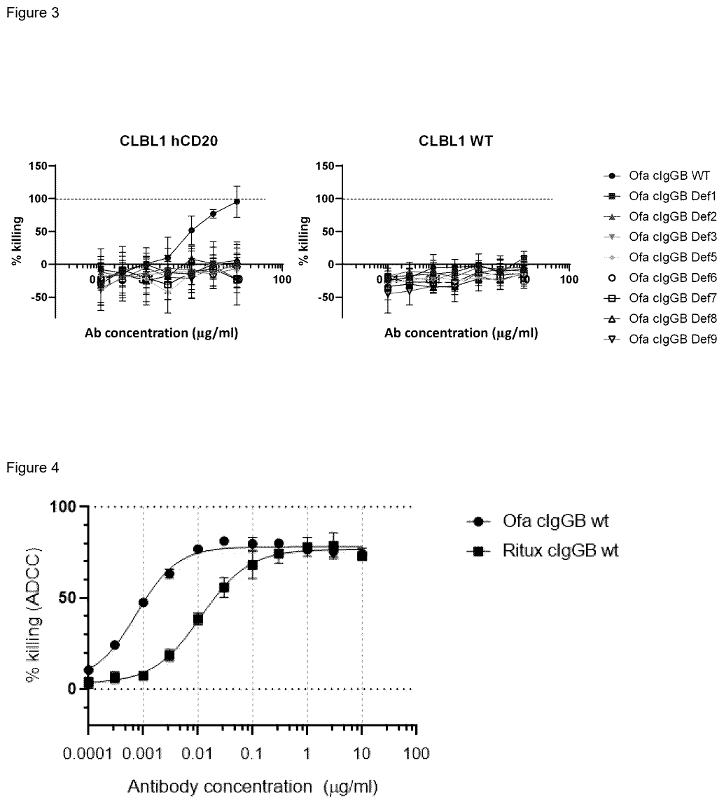

FIG. 3 shows the results of an exemplary CDC assay performed using hCD20 expressing CLBL1 cells and WT CLBL1 cells and canine IgG-B WT or containing mutations Def1, 2, 3, 5, 6, 7, 8, 9. Antibodies were used at serial 1:3 dilutions ranging from 10 μg/ml to 0.01 10 μg/ml. All Def mutants tested (Def1, 2, 3, 5, 6, 7, 8, 9) completely abrogated the ability of canine IgGB WT to kill hCD20 CLBL1 cells by complement dependent cytotoxicity (FIG. 3). All mutant variants tested show full suppression of CDC activity, suggesting that C1q interaction with Fc has stringent requirement of the sequence contexts from all three regions of the Fc, lower hinge, SHED area and the proline sandwich.

### 4. Antibody Dependent Cellular Cytotoxicity (ADCC) Activity

Canine cell lines, such as MDCK II cells (ATCC) were stably transfected or nucleofected with a construct encoding for the human CD20 protein and a construct expressing a fluorescent protein (i.e. GFP), as described above. Wild type MDCK II cells were used as a control. Canine peripheral blood mononuclear cells (PBMCs, Envigo) were used as a source of effector cells. PBMCs were isolated from freshly drawn whole blood, with sodium heparin anticoagulant, using Ficoll-Paque plus (Cytiva, GE17-1440-02) density gradient centrifugation. In brief, 10 ml blood was diluted 1:1 with phosphate buffer saline (PBS) and carefully layered on top of 15 ml of Ficoll-paque plus, centrifugated at 800rcf for 20′ with slow acceleration and no break at the end. The top layer and interphase disk were diluted with PBS and centrifuged at 1300 rpm for 10′ to collect PBMC in the pellet which was washed a second time in PBS to remove all remnants of Ficoll. After a second centrifugation, PBMCs were resuspended in media (PBMC media=RPMI+10% heat inactivated foetal bovine serum+1% penicillin-streptomycin+1% non-essential amino acids+1% L-glutamine+1% sodium pyruvate+1% HEPES) supplemented with 50 ng/ml recombinant canine IL-2 (R&D systems) and incubated for 24 h at 37° C., 5% CO₂ before use in the ADCC assay.

We first compared the ADCC activity of two well-known anti-human CD20 antibodies, Ofatumumab and Rituximab, formulated in WT canine IgG-B (FIG. 4). Our own data is comparable to published ADCC data generated for Ofatumumab and Rituximab formulated on human Fc. Ofatumumab: Bleeker W K, et al. Br J Haematol 2008; 140: 303-312. Rituximab: https://clean-cells.com/activities/bioassay-and-rd/cytotoxicity-assays/

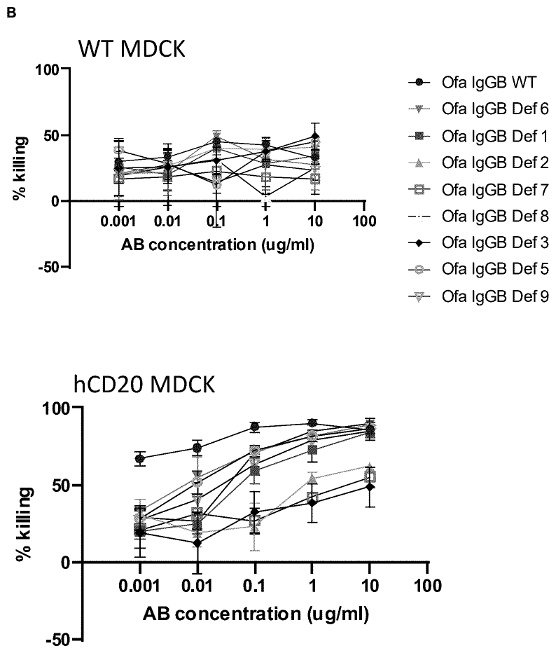

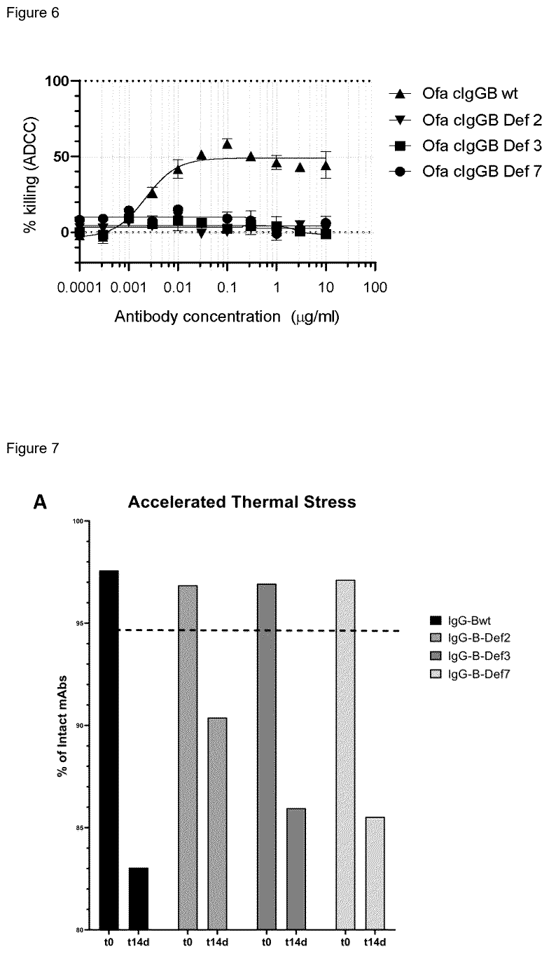

Some differences between assay setup and target cells are expected and acceptable. Next, we assessed the ADCC activity. To assess canine ADCC activity, 10,000 (FIG. 5, assay setup 1) or 5,000 cells (FIG. 6, assay setup 2) MDCK II cells wild type or expressing hCD20 were co-cultured with PBMCs at an effector:target ratio of 50:1 (assay setup 1) or 100:1 (assay setup 2) and a titration of antibodies, as shown in FIGS. 5 and 6, for 24 h at 37° C., 5% CO₂, in a 1:1 mix of MDCK II media (DMEM+1% L-glutamine+10% fetal bovine serum) and PBMC media.

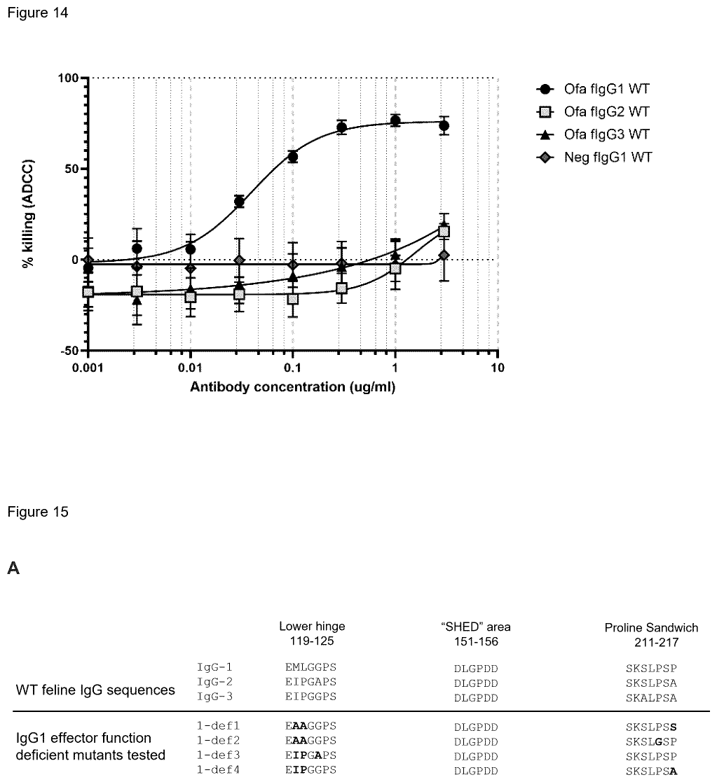

To assess feline ADCC activity, 10,000 (FIG. 14, assay setup 3) MDCK II cells wild type or expressing hCD20 were co-cultured with PBMCs at an effector:target ratio of 25:1 (assay setup 3) and a titration of antibodies, as shown in FIGS. 14, for 24 h at 37° C., 5% CO₂, in a 1:1 mix of MDCK II media (DMEM+1% L-glutamine+10% fetal bovine serum) and PBMC media.

GFP signal, which is proportional to the number of live cells per well, was used as a measure of the number of live cells remaining in the well at the end of the 24 h incubation. GFP signal was measured on a CLARIOstar (BMG Labtech). Data was analysed using MARS (BMG Labtech) and percentage of killing in the presence of antibodies was calculated using Microsoft Excel. Background signal was obtained from a sample of cells treated with 1% triton (where no cells were left alive) and subtracted from the signal obtained from each test sample. Max signal (0% killing) was obtained from a sample of cells treated identically but where antibodies were omitted. Graphs were plotted in Microsoft Excel or GraphPad Prism.

Canine Def mutants 2, 3 and 7 fully suppress IgG-B ADCC activity at up to 10 μg/ml. We observed partial effector function of the other mutants in this assay. Results are shown in FIG. 5 a) and b). FIG. 5 b) is a combination of the data shown in FIG. 5 a). The left panel shows the results obtained for the mutants using human CD20 MDCK cells and the right panel shows the results using wild type MDCK cells.

FIG. 5 not only demonstrates the degree of suppression of ADCC activity of individual canine mutant variant compared to wild type IgG-B, by comparing between the mutant variants, we can further derive knowledge on the sequence and function correlations on individual regions and mutations.

Def 1 VS Def 2: Def 2 has additional “SHED” area mutation compared to Def 1. In ADCC assay, Def 1 is partially inactive, while Def2 is fully deficient for ADCC activity. This suggests that while M120S in the lower hinge and N210H+K212I+A213G triple mutation in the proline sandwich region are important to inactivate ADCC activity, D153G+P154R double mutation is necessary for the complete inactivation of ADCC activity.

Def 2 VS Def 3: Def 3 has an additional mutation of P217S compared to Def 2. Both Def 2 and Def 3 show full deficiency of ADCC activity. This suggests that Def2 mutation combination is sufficient in inactivating ADCC function, while the effect of P217S could not be accessed in this combination.

Def 5 VS Def 6 and Def 7: Def 5 has the human equivalent “LALA” mutation, M120L+L121A in canine IgG-B term. Def 6 and Def 7 has an additional single mutation in the proline sandwich region, P217S and P215G respectively. Def 5 and Def 6 are both partially functional for ADCC activity to a similar extend, suggesting that the additional P217S mutation does not contribute to the ADCC activity suppression. In contrast, Def 7 fully suppresses ADCC activity compared to partial effect of Def5, suggesting that P215G plays a role in ADCC suppression.

Def 6 VS Def 7: Despite sharing the same M120L+L121A lower hinge mutation, distinct mutations within the proline sandwich area result in different functional outcome of these 2 mutants. As discussed earlier, P215G is important in suppressing ADCC activity, while P217S does not have an impact.

As no prior knowledge of WT feline IgG ADCC activity has been reported, all IgG isoforms were assessed for ADCC activity. As shown in FIG. 14, feline IgG1 was shown to be effector function proficient, whereas both IgG2 and IgG3 were effector function deficient with little ADCC activity observed at up to 3 μg/ml.

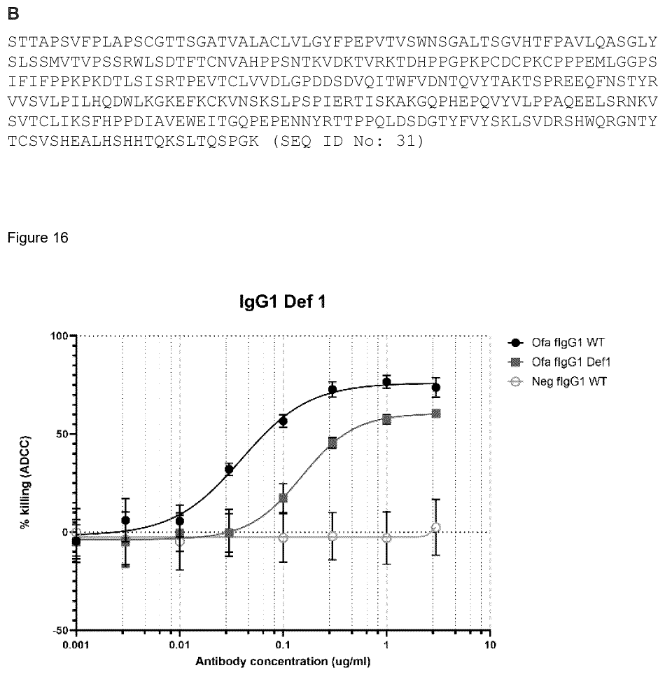

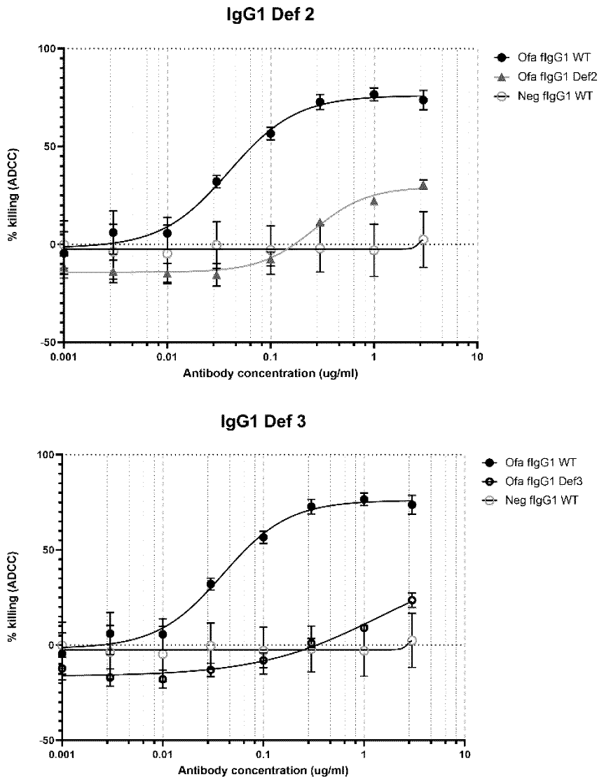

All feline Def mutant resulted in a reduction in effector function, with Def3 and Def2 strongly reduced IgG 1 ADCC activity to that level observed in both WT IgG2 and IgG3. Results are shown in FIG. 16. As in the dog, we can further derive knowledge on the sequence and function correlations on individual regions and mutations based on the results generated in FIG. 16.

Def 1 VS Def 2: Def 1 and Def 2 share the human equivalent “LALA” mutation, M120A+L121A in feline IgG 1 terms. They differ by an additional single mutation in the proline sandwich region, P217S and P215G respectively. They are thus equivalent to that of the canine Def 6 and Def 7 mutant variants respectively. Just like in the canine system, the additional P217S mutation had minimal contribution to the ADCC activity suppression, whereas P215G resulted in a 60% decrease in ADCC activity suggesting that P215G plays a role in ADCC suppression.

Def 3 VS Def 4: Def 3 and Def 4 similarly have amino acid mutations at positions M120 and L121, in feline IgG 1 terms, within the lower hinge region. Rather than utilising the “LALA” mutation, feline IgG 1 was reverted to that observed in the naturally effector function deficient feline IgG2 and IgG3 (FIG. 15A), M120I and L121P. Def 3 has an additional mutation of G123A within this lower hinge region akin to WT IgG2, whereas Def 4 retains G123 like that observed in both WT IgG1 and IgG3. Def 4 has an additional proline sandwich mutation P217A, like that observed in both WT IgG2 and IgG3. In the ADCC assay, Def 4 is partially inactive (32% decreased compared to WT), while Def 3 is on par to that observed with WT IgG2 and 3 (FIG. 14, FIG. 16), being deficient for ADCC activity. This suggests that the additional mutation of G123A within the lower hinge is necessary for the inactivation of ADCC activity, with modification of P217 again had minimal contribution to ADCC activity suppression.

Taken together, these observations demonstrated that the individual mutation and region contribution are not predictable from sequence alone in terms of the degree of ADCC function suppression. The fully functional deficient canine mutants, i.e. fully CDC and ADCC deficient are Def 2, 3, 7. As ADCC function was shown to be a more sensitive measure of effector function deficiency this alone was utilised with the feline mutants. The fully functional deficient feline mutants, i.e. ADCC deficient are Def2 and Def3.

### 5. Stability and Thermal Stress

6× 50 μL aliquots of canine IgG-B wt and Def2, Def3 and Def7 at 5 mg/mL in PBS were prepared and stored at either 4° C. (2 aliquots per molecule) or 40° C. (4 aliquots per molecule) for 1, 4 or 14 days in sealed PCR tubes.

HPLC-SEC chromatography (column: BioResolve SEC mAb 200A, 2.5 um column WATERS) was performed using ACQUITY H-class Bio from WATERS using PBS as mobile phase with isocratic 0.575 mL/min flow rate. At each timepoint (day 0, 1, 4 and 14), all the above-mentioned molecules have been centrifugated 5′ at 20000 g (using standard table top centrifuge) to remove any precipitates and then 10 μL of each sample have been injected in H-SEC using the above-mentioned protocol. Percentage of monomeric species and Area (indicative of antibody concentration) was determined for each molecule/timepoints and compared to a reference run (day 0). The results are summarised in FIG. 7A and show that the thermal stability of canine IgGB Def2, 3 and 7 are comparable to that of WT IgGB.

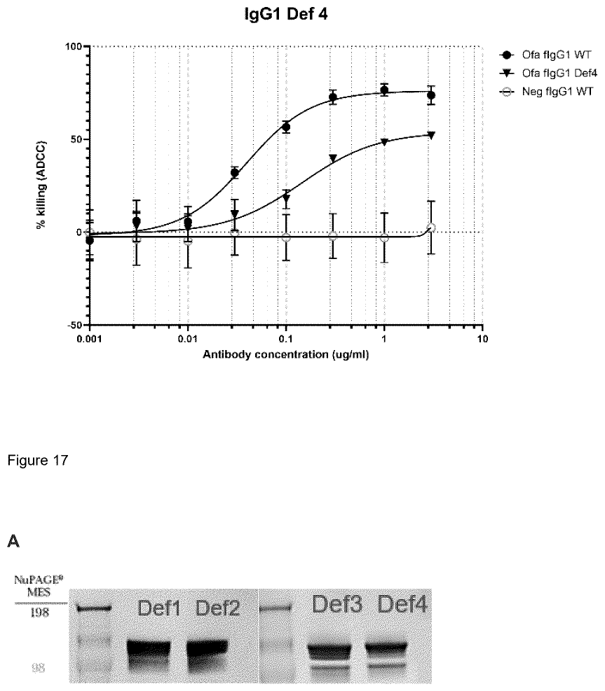

Feline IgG1 WT (FIG. 13B) and Def1, Def2, Def3 and Def4 mutants (FIG. 17A) were assessed by SDS-Page using 4-12% Bis-Tris NuPAGE gels, MES SDS running buffer and SeeBlue Plus 2 standards, all from ThermoFisher under non-reducing conditions. No clear differences were observed. HPLC-SEC chromatography was also performed using the method stated above for the IgG1 WT and Def mutants. FIG. 17B shows that all IgG1 WT and mutants showed a high percentage of intact monomeric species (above 96%).

### 6. Protein A Binding Affinity Validation

Purified canine antibodies (Ofatumumab canine IgG-B wt and deficient mutants “IgG-B wt” and “Def-2.”, “Def 3” and “Def 7” as described above, with reference to FIG. 2) in PBS were concentrated using centrifugal concentrators (Sartorious—VS02H22) to 5 mg/mL. Protein concentration was assessed using UV absorbance at 280 nm with NanoDrop™ One (Thermo Scientific™) using standard IgG parameters.

Binding affinity of IgGs to Protein A was assessed using Biacore 8K (Cytiva). Briefly, Sensor Chip Protein A (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

Antibodies dilutions were prepared diluting IgG-B WT, Def2, Def3 and Def7 from 1 uM to 4 nM (6 concentrations with 1:3 dilutions) in Running Buffer and kinetics was assessed using single cycle kinetics method (Biacore Assay Handbook, Cytiva). Kinetics and/or Affinity quantification have been performed using Biacore Insight following standard analyses methods.

The results show that protein A binding is maintained in canine IgG-B Def2, 3 and 7 (FIG. 7B top panel).

antibodies (Ofatumumab feline IgG1 WT and deficient mutants “IgG1 WT” and “Def1.”, “Def2”, “Def3” and “Def4” as described above, with reference to FIG. 15) binding affinity to Protein A was similarly assessed using Biacore 8K (Cytiva). Using the methods as described above for canine IgGs.

Antibodies dilutions were prepared by diluting IgG1 WT” and “Def1”, “Def2”, “Def3” and “Def4” from 500 nM to 31.25 nM (5 concentrations with 1:2 dilutions) in Running Buffer (10 mM HEPES pH7.4 150 mM NaCl 3 mM EDTA and 0.005% Tween20) and kinetics was assessed using single cycle kinetics method (Biacore Assay Handbook, Cytiva). Kinetics and/or Affinity quantification have been performed using Biacore Insight following standard analyses methods.

The results show that protein A binding is maintained in feline IgG1 Def 1, 2, 3 and 4 (FIG. 18A).

### 7. FcRn Binding Affinity Validation

Purified canine antibodies (Ofatumumab canine IgG-B wt and deficient mutants—“IgG-B wt” and “Def2”, “Def3” and “Def7” as described above, with reference to FIG. 2) in PBS were concentrated using centrifugal concentrators (Sartorious—VS02H22) to 5 mg/mL. Protein concentration was assessed using UV absorbance at 280 nm with NanoDrop™ One (Thermo Scientific™) using standard IgG parameters.

Binding affinity of canine IgGs to Fc Neonatal Receptor was assessed using Biacore 8K (Cytiva). Briefly, CM5 Sensor Chip (Cytiva) was docked into Biacore 8K, equilibrated for 30′ at RT and then Running Buffer (10 mM HEPES pH6 150 mM NaCl 3 mM EDTA and 0.005% Tween20) was applied to the SPR chip surface.

Canine FcRn-B2M recombinant protein (Immunitrack, ITF12) was diluted into 10 mM acetate buffer pH4.5 at 4 nM (1:2000 dilution from stock) and immobilised using standard amine coupling reaction.

Antibodies dilutions were prepared diluting IgG-B WT, Def2, Def3 and Def7 from 3 uM to 37 nM (5 concentrations with 1:3 dilutions) in Running Buffer and kinetics was assessed using multi-cycle kinetics method (120 sec association-300 sec dissociation). Kinetics and/or Affinity quantification have been performed using Biacore Insight following standard analyses methods.

Affinity to FcRn of IgG-B Def2, 3 and 7 was comparable to that of IgG-B WT (FIG. 7B bottom panel).

Feline Antibodies (Ofatumumab feline IgG1 WT and deficient mutants “IgG1 WT” and “Def1.”, “Def2”, “Def3” and “Def4” as described above, with reference to FIG. 15) binding affinity to Fc Neonatal Receptor was similarly assessed using Biacore 8K (Cytiva). Briefly, CAP Series S Sensor Chip (Cytiva) was docked into Biacore 8K, equilibrated overnight at RT and then Running Buffer (1×PBS and 0.05% Tween20) was applied to the SPR chip surface.

Feline FcRn-B2M recombinant protein (Acro Biosystems) was diluted into 0.5 ug/mL in running buffer and immobilised using the Biotin CAPture kit standard protocol.

Antibodies dilutions were prepared diluting IgG1 WT, Def1, Def2, Def3 and Def4 from 25 nM to 0.781 nM (6 concentrations with 1:2 dilutions) in Running Buffer and kinetics was assessed using multi-cycle kinetics method (60 sec association-120 sec dissociation). Kinetics and/or Affinity quantification have been performed using Biacore Insight following standard analyses methods.

Affinity to FcRn of feline IgG1 Def1,2, 3 and 4 was comparable to that of IgG1 WT (FIG. 18B).

### 8. FcγR1a (CD64) Binding Affinity Determination

Purified canine Antibodies (Ofatumumab canine IgG-B WT and deficient mutants—“IgG-B wt” and “Def1 to “Def8” as described above, with reference to FIG. 2) in PBS were normalised to 0.5 mg/mL, Protein concentration was assessed using UV absorbance at 280 nm with NanoDrop™ One (Thermo Scientific™) using standard IgG parameters.

Binding affinity of canine IgGs to Fc gamma 1 Receptor (CD64) was assessed using Biacore 8K (Cytiva).

Briefly, Protein A Sensor Chip (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

Ofatumumab canine IgG-B WT or Def mutants were diluted into running buffer at 6 nM concentration. These have been immobilised using 90 sec association at 10 uL/min as capturing step, followed by injection of running buffer to remove any unbound product.

Human CD64/FCGR1A Protein (from Stratech—Catalogue Number: 10256-H08H-SIB) was diluted in Running Buffer at 150 nM with 1:2 further dilutions down to 9.375 nM. Kinetics were assessed using multi-cycle kinetics with capture step method (60 sec association-450 sec dissociation) followed by regeneration step (0.1M Glycine pH2.2 contact time 60 sec FR 30 uL/min). Kinetics quantification have been performed using Biacore Insight following standard analyses methods.

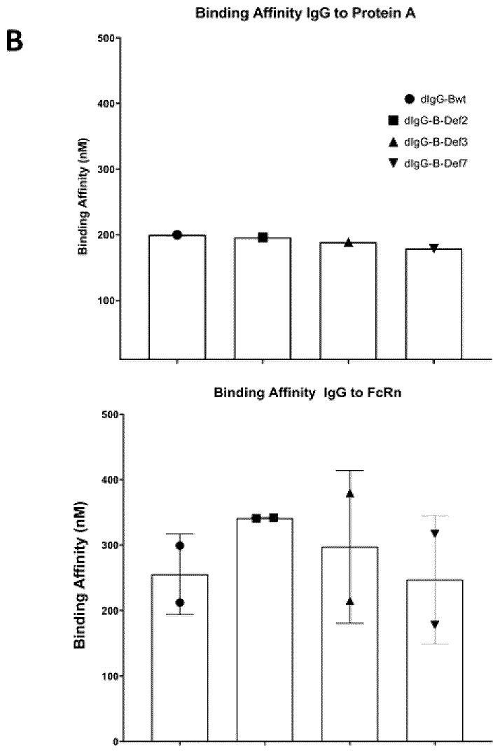

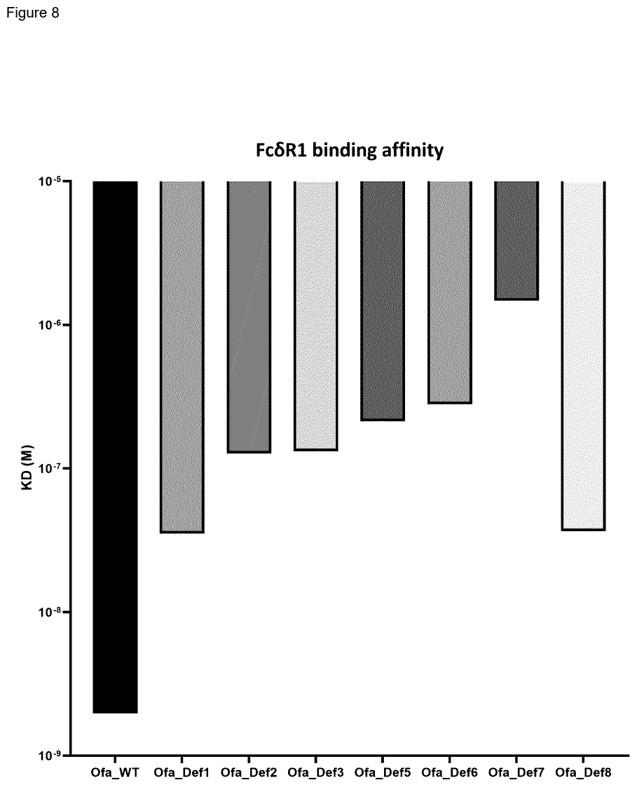

As a confirmation of the reduced CDC activity of Def mutants, affinity for FcγR1a of IgG-B Def1, 2, 3, 5, 6, 7 and 8 was reduced compared to that of WT IgGB (FIG. 8).

Feline Antibodies (Ofatumumab feline IgG WT and deficient mutants—“IgG1 WT”, “IgG2 WT”, “IgG3 WT”, and “Def1 to “Def4” as described above, with reference to FIG. 15) binding affinity to Fc gamma 1 Receptor (CD64) was similarly assessed using Biacore 8K (Cytiva), as per the methods stated in dog. As a confirmation of the reduced effector function activity of Def mutants, affinity for FcγR1a of IgG1 Def1, 2, 3 and 4 was reduced compared to that of WT IgG1 (FIG. 18C).

Considered together, although FcγR1a interaction correlates with effector function, i.e. functional proficiency tend to have lower FcγR1a affinity. However, FcγR1a interaction cannot predict the fully ADCC null mutant variants in both canine and feline cases demonstrated in FIG. 8 and FIG. 18C.

### 8. Antibody Dependent Cell Mediated Phagocytosis (ADCP) Activity Validation of Mutant Variants

A flow cytometry based ADCP assay could be developed based on equivalent protocols described for testing ADCP function of human monoclonal antibodies. Canine and feline phagocytic cells expressing FcγRIIa would be identified and used as effector cells. These could be primary canine or feline macrophages differentiated from PBMC derived monocytes by adapting protocols established for human monocyte derived macrophages (Jin and Kruth, J Vis Exp(112); 2016 54244), or a canine or feline macrophage cell line like DH82 (ATCC) or Fcwf-4 (ATCC) respectively. Suitable targets could be cell lines, like CLBL-1, MDCK II or CRFK, expressing antigen of interest, for e.g. hCD20, or beads/particles (i.e. viral-like particles) coated with the antigen. Effectors and targets would be distinguished by labelling them with a suitable combination of different fluorescence (e.g. GFP, RFP) that can be measured on a flow cytometer.

Assay conditions, like effector:target ratio, duration of culture, preactivation of effector cells, etc., would be optimised. Effector and target cells would be co-cultured in the presence of a titration of antibodies in optimised assay conditions. Absence of antibody or an appropriate isotype control antibody would be used as a negative control. Fluorescence of effector cells would be measured using a suitable flow cytometer and data would be analysed using FlowJo software. Percentage of ADCP i.e. phagocytosis would be determined as the percentage of effector cells (gating on live, single cells) that have become positive for target cell associated fluorescence.

### 9. FcRn Binding Validation of Canine Mutant Variants

Given the fact that canine IgG-B is able to bind to murine FcRn, In vivo half-life validation of mutant variants can be performed in immunocompromised mice (i.e. RAG1^{KO} mice). Canine IgG-B wild type and Def mutant antibodies are injected (intraperitoneal or intravenous) into such mice and serum titre of the injected antibodies can be measured over time. A small amount of blood (20-50 ul) would be collected from saphenous vein at different time points (i.e. 7 days before injection, 10 min, 24 h, 72 h, 7 days, 14 days, 21 days and 28 days after injection). Serum would be separated from cellular components by centrifuging the blood (7000 rcf for 5′). Canine IgGs in mouse serum could be quantified by using anti canine IgG ELISA (i.e. Abcam ab193768). This would enable pharmacokinetic profile (pK) to be determined.

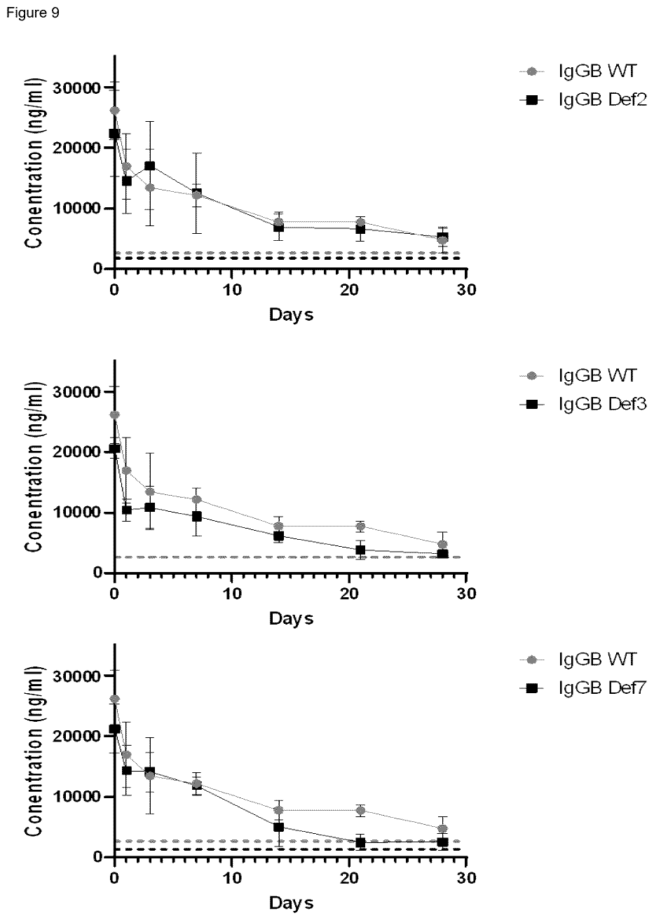

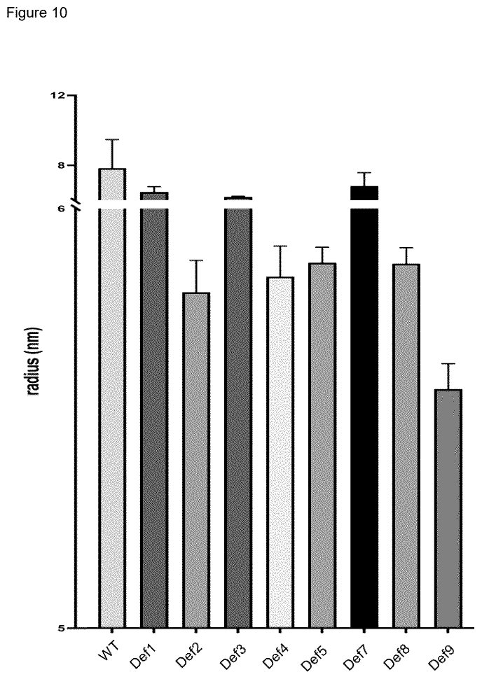

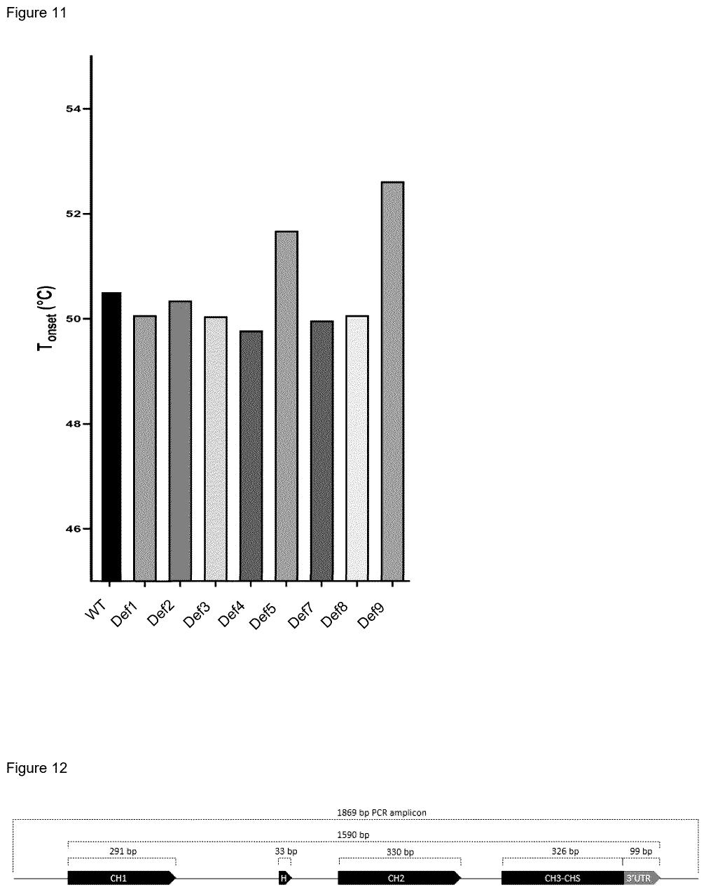

In the example shown in FIG. 9, Rag1 KO mice were injected intravenously with either Ofa-cIgGB-WT, Ofa-cIgGB-Def2, Ofa-cIgGB-Def3 or Ofa-cIgGB-Def7 (1 mg/Kg, 5 mice per antibody). Serum was produced from whole blood collected 30 min, 1 day, 3 days, 1 week, 2 weeks, 3 weeks and 4 weeks after antibody injection. To determine background levels of detection, blood was also collected a week before antibody injection. Canine IgG serum titre was determined using the anti-canine IgG ELISA kit (Biorbyt). ELISA plates were read at 450 nm with 650 nm correction using a CLARIOstar plate reader (BMG). Sera concentration in serum was calculated using the MARS software (BMG) using a standard curve present in the kit and multiplying for the dilution factor. Data were analysed using GraphPad.

### 10. Tonset and Radius Measurement

Purified canine Antibodies (Ofatumumab canine IgG-B wt and deficient mutants—“IgG-B wt” and “Def1 to “Def8” as described above, with reference to FIG. 2) in PBS were normalised to 5 mg/mL, Protein concentration was assessed using UV absorbance at 280 nm with NanoDrop™ One (Thermo Scientific™) using standard IgG parameters.

DynaPro® (WYATT) Plate Reader III has been used to estimate size of these molecules using Dynamic Light Scattering experiment as well as monitoring temperature induced aggregation, to highlight any protein folding destabilisation induced by deficient mutants.

Briefly, 10 uL of each Antibody were added to a 96 well plate and placed into plate reader equilibrated at RT. Each DLS measurements consisted of 10×10 seconds acquisition. Same parameters were used for temperature ramp experiment with a ΔT in ° C.=+1° C. per minute. Measurements were evaluated by DYNAMICS software. Values were analysed and plotted using GraphPad

The general stabilities of feline IgG1 WT and Def mutants 1,2,3 and 4, including unfolding and aggregation, were evaluated with the UNcle stability platform (Unchained Labs, Norton, MA). Differential scanning fluorimetry (DSF) and dynamic light scattering (DLS) were used to monitor the changes of protein size and structural distribution following a temperature ramp of 2° C./min from 25° C. to 95° C. Totally 9 μL of each sample was loaded into the sample well for DLS and intrinsic fluorescence test with triplicates. Measurements were evaluated using the UNcle analysis platform (Unchained Labs, Norton, MA).

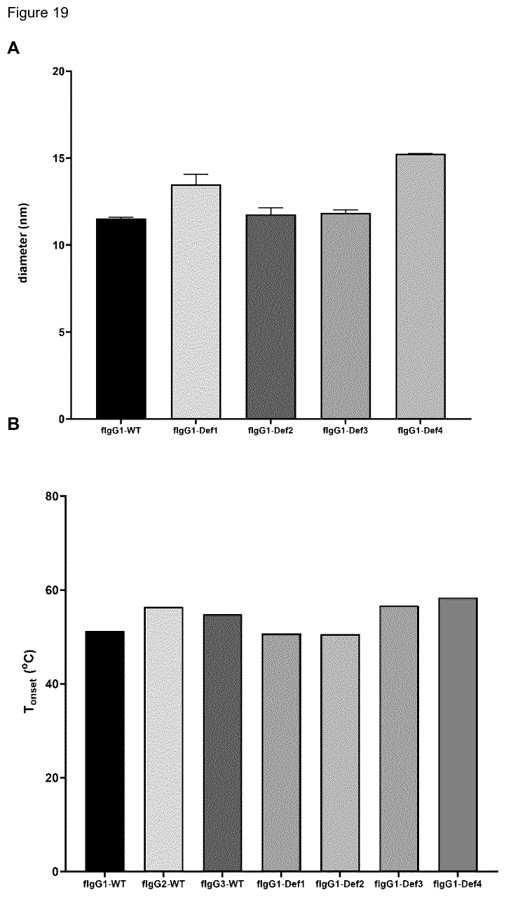

Feline Antibodies (Ofatumumab feline IgG1 WT and deficient mutants “IgG1 WT” and “Def1”, “Def2”, “Def3” and “Def4” as described above, with reference to FIG. 15) showed minimal changes in both Tonset and diameter measurements (FIGS. 19 A and B).

### 11. Discussion

Although macroscopically IgGs from different species are largely conserved in terms of structure, there is increasing evidence that differences in sequence context from different species do impact properties of the IgGs. Based on structure conservation between human and canine or feline IgGs, we were able to map the positions around the lower hinge, proline sandwich and SHED area which interact with C1q and FcR receptors, and are the main interacting partners of IgGs to mediate effector function. Although different mutation combinations have been identified in human for effector function modulation, the nature, position and combination of mutations are important to achieve fully effector function deficiency and is species dependent. Therefore, prediction alone based on primary sequence can lead to the identification of positions influencing effector function, but it is not sufficient to predict the mutation combinations which give rise to fully effector function deficient variant. Furthermore, FcγR1a interaction affinity of Fc is not sufficient to predict the fully effector function deficient variants, highlighting that experimentation on CDC and ADCC activity in vitro (and in vivo if possible) is the only reliable way to determine effector function deficiency.

Our design strategies are based on knowledge from the human domain and the characteristics of natural IgG isoforms within the species. The fact that all mutation variants have a level of influence on effector function clearly suggests that conservation between species and isoforms can guide the design and can predict to a degree the influence of sequence on function. However, in the canine case out of all the designs for example, only Def 2,3,7 are fully effector function deficient demonstrating that this outcome is not predictable and require the validation using stringent functional assessment. Similarly, in the feline case, utilising the design strategies used to generate effector function deficient IgGs in both human and canine systems results in only one of the four mutants tested being fully function deficient. For the feline case, this further emphasises the importance of function validation rather than using predictions from sequence conservation alone.

In addition to the function perspective, when mutations are introduced to an antibody sequence, other properties will need to be evaluated, such as its biophysical properties and binding capacity to protein A and neonatal receptors (FcRn). These properties may be altered with introduced mutations and often unpredictable as mutations can cause confirmational alterations to antibody structures which could affect regions other than the region of modification. To this end, we have extensively characterised our canine IgG-B mutation variants, especially Def2,3,7, with effector function (CC and ADCC) fully disabled, in terms of their biophysical properties (FIG. 7) and protein A and FcRn binding capacity (FIG. 8). For feline, mutant variants, we similarly have characterised the mutant variants in terms of their biophysical properties (FIG. 16) and protein A, FcgammaR1 and FcRn binding capacity (FIG. 17) Our data demonstrated that our mutation variants do not alter these properties, which is an important parameter to consider in the research and therapeutics context. Based on the result of these characteristics, we argue that the mutant variants we discovered are inventive, not predictable based on existing canine, feline or human literature.

TABLE 1
SEQUENCES

| SEQ ID | | |
|---|---|---|
| NO: | Description | Sequence |
| 1 | Ofa-VH-cIgGB | MELGLSWIFLLAILKGVQCEVQLVESGGGLVQPGRSLRLSCAAS |
| | WT: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKV |
| | | DKPVPKRENGRVPRPPDCPKCPAPEMLGGPSVFIFPPKPKDTL |
| | | LIARTPEVTCVVVDLDPEDPEVQISWFVDGKQMQTAKTQPREE |
| | | QFNGTYRVVSVLPIGHQDWLKGKQFTCKVNNKALPSPIERTISK |
| | | ARGQAHQPSVYVLPPSREELSKNTVSLTCLIKDFFPPDIDVEWQ |
| | | SNGQQEPESKYRTTPPQLDEDGSYFLYSKLSVDKSRWQRGDT |
| | | FICAVMHEALHNHYTQKSLSHSPGK |
| 2 | Ofa-VH-cIgGB- | MELGLSWIFLLAILKGVQCEVQLVESGGGLVQPGRSLRLSCAAS |
| | Def1: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APESLGGPSVFIFPPKPKDTLLIARTPEVTCVVVDLDPEDPEVQI |
| | | SWFVDGKQMQTAKTQPREEQFNGTYRVVSVLPIGHQDWLKGK |
| | | QFTCKVNHIGLPSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 3 | Ofa-VH-cIgGB- | MELGLSWIFLLAILKGVQCEVQLVESGGGLVQPGRSLRLSCAAS |
| | Def2: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APESLGGPSVFIFPPKPKDTLLIARTPEVTCVVVDLGREDPEVQI |
| | | SWFVDGKQMQTAKTQPREEQFNGTYRVVSVLPIGHQDWLKGK |
| | | QFTCKVNHIGLPSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 4 | Ofa-VH-cIgGB- | MELGLSWIFLLAILKGVQCEVQLVESGGGLVQPGRSLRLSCAAS |
| | Def3: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APESLGGPSVFIFPPKPKDTLLIARTPEVTCVVVDLGREDPEVQI |
| | | SWFVDGKQMQTAKTQPREEQFNGTYRVVSVLPIGHQDWLKGK |
| | | QFTCKVNHIGLPSSIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 5 | Ofa-VH- | MELGLSWIFLLAILKGVQCEVQLVESGGGLVQPGRSLRLSCAAS |
| | cIgGB-Def4: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APESLGGPSVFIFPPKPKDTLLIARTPEVTCVVVDLGREDPEVQI |
| | | SWFVDGKQMQTAKTQPREEQFNGTYRVVSVLPIGHQDWLKGK |
| | | QFTCKVNHIGLGSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 6 | Ofa-VH-cIgGB- | MELGLSWIFLLAILKGVQCEVQLVESGGGLVQPGRSLRLSCAAS |
| | Def5: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APEAAGGPSVFIFPPKPKDTLLIARTPEVTCVVVDLDPEDPEVQI |
| | | SWFVDGKQMQTAKTQPREEQFNGTYRVVSVLPIGHQDWLKGK |
| | | QFTCKVNNKALPSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 7 | Ofa-VH-cIgGB- | MELGLSWIFLLAILKGVQCEVQLVESGGGLVQPGRSLRLSCAAS |
| | Def6: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APEAAGGPSVFIFPPKPKDTLLIARTPEVTCVVVDLDPEDPEVQI |
| | | SWFVDGKQMQTAKTQPREEQFNGTYRVVSVLPIGHQDWLKGK |
| | | QFTCKVNNKALPSSIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 8 | Ofa-VH-cIgGB- | MELGLSWIFLLAILKGVQCEVQLVESGGGLVQPGRSLRLSCAAS |
| | Def7: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APEAAGGPSVFIFPPKPKDTLLIARTPEVTCVVVDLDPEDPEVQI |
| | | SWFVDGKQMQTAKTQPREEQFNGTYRVVSVLPIGHQDWLKGK |
| | | QFTCKVNNKALGSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 9 | Ofa-VH-cIgGB- | MELGLSWIFLLAILKGVQCEVQLVESGGGLVQPGRSLRLSCAAS |
| | Def8: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APGMLGGPSVFIFPPKPKDTLLIARTPEVTCVVVDLDPENPEVQI |
| | | SWFVDGKQMQTAKTQPREEQFNGTYRVVSVLPIGHQDWLKGK |
| | | QFTCKVNNKALPSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 10 | Ofa-VH-cIgGB- | MELGLSWIFLLAILKGVQCEVQLVESGGGLVQPGRSLRLSCAAS |
| | Def9: | GFTFNDYAMHWVRQAPGKGLEWVSTISWNSGSIGYADSVKGR |
| | | FTISRDNAKKSLYLQMNSLRAEDTALYYCAKDIQYGNYYYGMD |
| | | VWGQGTTVTVSSASTTAPSVFPLAPSCGSTSGSTVALACLVSG |
| | | YFPEPVTVSWNSGSLTSGVHTFPSVLQSSGLYSLSSMVTVPSS |
| | | RWPSETFTCNVAHPASKTKVDKPVPKRENGRVPRPPDCPKCP |
| | | APEAAGGPSVFIFPPKPKDTLLIARTPEVTCVVVDLDPENPEVQI |
| | | SWFVDGKQMQTAKTQPREEQFNGTYRVVSVLPIGHQDWLKGK |
| | | QFTCKVNNKALPSSIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 11 | cIgGB wt | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | | HPASKTKVDKPVPKRENGRVPRPPDCPKCPAPEMLGGPSVFIF |
| | | PPKPKDTLLIARTPEVTCVVVDLDPEDPEVQISWFVDGKQMQTA |
| | | KTQPREEQFNGTYRVVSVLPIGHQDWLKGKQFTCKVNNKALPS |
| | | PIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 12 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region ,Def1 | HPASKTKVDKPVPKRENGRVPRPPDCPKCPAPESLGGPSVFIF |
| | mutant: | PPKPKDTLLIARTPEVTCVVVDLDPEDPEVQISWFVDGKQMQTA |
| | | KTQPREEQFNGTYRVVSVLPIGHQDWLKGKQFTCKVNHIGLPS |
| | | PIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 13 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def2 | HPASKTKVDKPVPKRENGRVPRPPDCPKCPAPESLGGPSVFIF |
| | mutant: | PPKPKDTLLIARTPEVTCVVVDLGREDPEVQISWFVDGKQMQT |
| | | AKTQPREEQFNGTYRVVSVLPIGHQDWLKGKQFTCKVNHIGLP |
| | | SPIERTISKARGQAHQPSVYVLPPSREELSKNTVSLTCLIKDFFP |
| | | PDIDVEWQSNGQQEPESKYRTTPPQLDEDGSYFLYSKLSVDKS |
| | | RWQRGDTFICAVMHEALHNHYTQKSLSHSPGK |
| 14 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def3 | HPASKTKVDKPVPKRENGRVPRPPDCPKCPAPESLGGPSVFIF |
| | mutant: | PPKPKDTLLIARTPEVTCVVVDLGREDPEVQISWFVDGKQMQT |
| | | AKTQPREEQFNGTYRVVSVLPIGHQDWLKGKQFTCKVNHIGLP |
| | | SSIERTISKARGQAHQPSVYVLPPSREELSKNTVSLTCLIKDFFP |
| | | PDIDVEWQSNGQQEPESKYRTTPPQLDEDGSYFLYSKLSVDKS |
| | | RWQRGDTFICAVMHEALHNHYTQKSLSHSPGK |
| 15 | Modified | ASTTAPSVFPLAPSCGSTSGSTVALACLVSGYFPEPVTVSWNS |
| | cIgGB constant | GSLTSGVHTFPSVLQSSGLYSLSSMVTVPSSRWPSETFTCNVA |
| | region region, | HPASKTKVDKPVPKRENGRVPRPPDCPKCPAPESLGGPSVFIF |
| | Def4 mutant: | PPKPKDTLLIARTPEVTCVVVDLGREDPEVQISWFVDGKQMQT |
| | | AKTQPREEQFNGTYRVVSVLPIGHQDWLKGKQFTCKVNHIGLG |
| | | SPIERTISKARGQAHQPSVYVLPPSREELSKNTVSLTCLIKDFFP |
| | | PDIDVEWQSNGQQEPESKYRTTPPQLDEDGSYFLYSKLSVDKS |
| | | RWQRGDTFICAVMHEALHNHYTQKSLSHSPGK |
| 16 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def5 | HPASKTKVDKPVPKRENGRVPRPPDCPKCPAPEAAGGPSVFIF |
| | mutant: | PPKPKDTLLIARTPEVTCVVVDLDPEDPEVQISWFVDGKQMQTA |
| | | KTQPREEQFNGTYRVVSVLPIGHQDWLKGKQFTCKVNNKALPS |
| | | PIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 17 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def6 | HPASKTKVDKPVPKRENGRVPRPPDCPKCPAPEAAGGPSVFIF |
| | mutant: | PPKPKDTLLIARTPEVTCVVVDLDPEDPEVQISWFVDGKQMQTA |
| | | KTQPREEQFNGTYRVVSVLPIGHQDWLKGKQFTCKVNNKALPS |
| | | SIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 18 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def7 | HPASKTKVDKPVPKRENGRVPRPPDCPKCPAPEAAGGPSVFIF |
| | mutant: | PPKPKDTLLIARTPEVTCVVVDLDPEDPEVQISWFVDGKQMQTA |
| | | KTQPREEQFNGTYRVVSVLPIGHQDWLKGKQFTCKVNNKALG |
| | | SPIERTISKARGQAHQPSVYVLPPSREELSKNTVSLTCLIKDFFP |
| | | PDIDVEWQSNGQQEPESKYRTTPPQLDEDGSYFLYSKLSVDKS |
| | | RWQRGDTFICAVMHEALHNHYTQKSLSHSPGK |
| 19 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def8 | HPASKTKVDKPVPKRENGRVPRPPDCPKCPAPGMLGGPSVFIF |
| | mutant: | PPKPKDTLLIARTPEVTCVVVDLDPENPEVQISWFVDGKQMQTA |
| | | KTQPREEQFNGTYRVVSVLPIGHQDWLKGKQFTCKVNNKALPS |
| | | PIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 20 | Modified cIgGB | ASTTAPSVFPLAPSCGSTSGSTVALACLVSGYFPEPVTVSWNS |
| | constant region | GSLTSGVHTFPSVLQSSGLYSLSSMVTVPSSRWPSETFTCNVA |
| | region, Def9 | HPASKTKVDKPVPKRENGRVPRPPDCPKCPAPEAAGGPSVFIF |
| | mutant: | PPKPKDTLLIARTPEVTCVVVDLDPENPEVQISWFVDGKQMQTA |
| | | KTQPREEQFNGTYRVVSVLPIGHQDWLKGKQFTCKVNNKALPS |
| | | SIERTISKARGQAHQPSVYVLPPSREELSKNTVSLTCLIKDFFPP |
| | | DIDVEWQSNGQQEPESKYRTTPPQLDEDGSYFLYSKLSVDKSR |
| | | WQRGDTFICAVMHEALHNHYTQKSLSHSPGK |
| 21 | IgG-B | MEFVLGWVFLVAILQGVQGEVQLVESGGDLVKPAGSLRLSCVA |
| | | SGFTFSNNAMNWVRQAPGKGLQWVAGINSGGSTASADAVKG |
| | | RFTISRDNAKNTVYLQMNSLTAEDTAVYYCAKVIGNWIATSDLD |
| | | YWGQGTLVIVSSASTTAPSVFPLAPSCGSTSGSTVALACLVSGY |
| | | FPEPVTVSWNSGSLTSGVHTFPSVLQSSGLYSLSSMVTVPSSR |
| | | WPSETFTCNVAHPASKTKVDKPVPKRENGRVPRPPDCPKCPA |
| | | PEMLGGPSVFIFPPKPKDTLLIARTPEVTCVVVDLDPEDPEVQIS |
| | | WFVDGKQMQTAKTQPREEQFNGTYRVVSVLPIGHQDWLKGK |
| | | QFTCKVNNKALPSPIERTISKARGQAHQPSVYVLPPSREELSKN |
| | | TVSLTCLIKDFFPPDIDVEWQSNGQQEPESKYRTTPPQLDEDG |
| | | SYFLYSKLSVDKSRWQRGDTFICAVMHEALHNHYTQKSLSHSP |
| | | GK |
| 22 | IgG-A | MEFVLGWVFLVAILQGVQGEVQLVESGGDLVKPAGSLRLSCVA |
| | | SGFTFSNNAMNWVRQAPGKGLQWVAGINSGGSTASADAVKG |
| | | RFTISRDNAKNTVYLQMNSLTAEDTAVYYCAKVIGNWIATSDLD |
| | | YWGQGTLVIVSSASTTAPSVFPLAPSCGSTSGSTVALACLVSGY |
| | | FPEPVTVSWNSGSLTSGVHTFPSVLQSSGLYSLSSMVTVPSSR |
| | | WPSETFTCNVVHPASNTKVDKPVFNECRCTDTPPCPVPEPLG |
| | | GPSVLIFPPKPKDILRITRTPEVTCVVLDLGREDPEVQISWFVDG |
| | | KEVHTAKTQSREQQFNGTYRVVSVLPIEHQDWLTGKEFKCRVN |
| | | HIDLPSPIERTISKARGRAHKPSVYVLPPSPKELSSSDTVSITCLI |
| | | KDFYPPDIDVEWQSNGQQEPERKHRMTPPQLDEDGSYFLYSK |
| | | LSVDKSRWQQGDPFTCAVMHETLQNHYTDLSLSHSPGK |
| 23 | IgG-C | MEFVLGWVFLVAILQGVQGEVQLVESGGDLVKPAGSLRLSCVA |
| | | SGFTFSNNAMNWVRQAPGKGLQWVAGINSGGSTASADAVKG |
| | | RFTISRDNAKNTVYLQMNSLTAEDTAVYYCAKVIGNWIATSDLD |
| | | YWGQGTLVIVSSASTTAPSVFPLAPSCGSQSGSTVALACLVSG |
| | | YIPEPVTVSWNSGSLTSGVHTFPSILQSSGLYSLSSMVTVPSSR |
| | | WPSETFTCNVAHPATNTKVDKPVVKECECKCNCNNCPCPGCG |
| | | LLGGPSVFIFPPKPKDILVTARTPTVTCVVVDLDPENPEVQISWF |
| | | VDSKQVQTANTQPREEQSNGTYRVVSVLPIGHQDWLSGKQFK |
| | | CKVNNKALPSPIEEIISKTPGQAHQPNVYVLPPSRDEMSKNTVT |
| | | LTCLVKDFFPPEIDVEWQSNGQQEPESKYRMTPPQLDEDGSYF |
| | | LYSKLSVDKSRWQRGDTFICAVMHEALHNHYTQKSLSHSPGK |
| 24 | IgG-D | MEFVLGWVFLVAILQGVQGEVQLVESGGDLVKPAGSLRLSCVA |
| | | SGFTFSNNAMNWVRQAPGKGLQWVAGINSGGSTASADAVKG |
| | | RFTISRDNAKNTVYLQMNSLTAEDTAVYYCAKVIGNWIATSDLD |
| | | YWGQGTLVIVSSASSTAPSVFPLAPSCGSTSGSTVALACLVSG |
| | | YFPEPVTVSWNSGSLTSGVHTFPSVLKSSGLYSLSSMVTVPSS |
| | | RLPSETFTCNVVHPATNTKVDKPVPKESTCKCISPCPVPESLGG |
| | | PSVFIFPPKPKDILRITRTPEVTCVVLDLGREDPEVQISWFVDGK |
| | | EVHTAKTQPREQQFNSTYRVVSVLPIEHQDWLTGKEFKCRVNH |
| | | IGLPSPIERTISKARGQAHQPGVYVLPPSPKELSSSDTVTLTCLIK |
| | | DFFPPEIDVEWQSNGQPEPESKYHTTAPQLDEDGSYFLYSKLS |
| | | VDKSRWQQGDPFTCAVMHEALQNHYTDLSLSHSPGK |
| 25 | hCD20 DNA | 5′ |
| | sequence used | ATGACAACACCCAGAAATTCAGTAAATGGGACTTTCCCGGCA |
| | for generation of | GAGCCAATGAAAGGCCCTATTGCTATGCAATCTGGTCCAAAA |
| | target cells: | CCACTCTTCAGGAGGATGTCTTCACTGGTGGGCCCCACGCA |
| | | AAGCTTCTTCATGAGGGAATCTAAGACTTTGGGGGCTGTCCA |
| | | GATTATGAATGGGCTCTTCCACATTGCCCTGGGGGGTCTTCT |
| | | GATGATCCCAGCAGGGATCTATGCACCCATCTGTGTGACTGT |
| | | GTGGTACCCTCTCTGGGGAGGCATTATGTATATTATTTCCGG |
| | | ATCACTCCTGGCAGCAACGGAGAAAAACTCCAGGAAGTGTTT |
| | | GGTCAAAGGAAAAATGATAATGAATTCATTGAGCCTCTTTGC |
| | | TGCCATTTCTGGAATGATTCTTTCAATCATGGACATACTTAAT |
| | | ATTAAAATTTCCCATTTTTTAAAAATGGAGAGTCTGAATTTTAT |
| | | TAGAGCTCACACACCATATATTAACATATACAACTGTGAACCA |
| | | GCTAATCCCTCTGAGAAAAACTCCCCATCTACCCAATACTGT |
| | | TACAGCATACAATCTCTGTTCTTGGGCATTTTGTCAGTGATG |
| | | CTGATCTTTGCCTTCTTCCAGGAACTTGTAATAGCTGGCATC |
| | | GTTGAGAATGAATGGAAAAGAACGTGCTCCAGACCCAAATCT |
| | | AACATAGTTCTCCTGTCAGCAGAAGAAAAAAAAGAACAGACT |
| | | ATTGAAATAAAAGAAGAAGTGGTTGGGCTAACTGAAACATCT |
| | | TCCCAACCAAAGAATGAAGAAGACATTGAAATTATTCCAATC |
| | | CAAGAAGAGGAAGAAGAAGAAACAGAGACGAACTTTCCAGA |
| | | ACCTCCCCAAGATCAGGAATCCTCACCAATAGAAAATGACAG |
| | | CTCTCCTTAA |
| | | 3′ |
| 26 | Nucleotide | ATGACAACACCCAGAAATTCAATGAGTGGAACTCTCCCGGTA |
| | sequence of | GATCCTATGAAAAGCCCTACTGCCATGTATCCTGTTCAAAAA |
| | canine CD20 | ATAATTCCCAAAAGGATGCCTTCAGTGGTGGGCCCTACACAA |
| | coding region | AACTTCTTCATGAGGGAATCTAAGACACTGGGGGCTGTCCAG |
| | | ATTATGAATGGGCTCTTCCACATTGCCCTAGGCAGCCTCCTG |
| | | ATGATTCACACGGATGTCTATGCGCCCATCTGTATAACTATG |
| | | TGGTACCCTCTCTGGGGAGGCATTATGTTCATCATTTCTGGA |
| | | TCACTCCTGGCAGCAGCGGACAAAAACCCCAGGAAGAGTTT |
| | | GGTCAAAGGAAAAATGATAATGAACTCATTGAGCCTCTTTGC |
| | | TGCTATTTCTGGAATAATTTTTTTGATCATGGACATATTTAATA |
| | | TTACCATTTCCCATTTTTTTAAAATGGAGAATTTGAATCTTATT |
| | | AAAGCTCCCATGCCATATGTTGACATACACAACTGTGACCCA |
| | | GCTAACCCCTCTGAGAAAAACTCTTTATCTATACAATATTGTG |
| | | GCAGCATACGATCTGTTTTCTTGGGCGTTTTTGCTGTGATGG |
| | | TGATCTTTACCTTTTTCCAGAAACTTGTGACAGCTGGCATTGT |
| | | TGAGAATGAATGGAAAAAACTGTGCTCTAAACCTAAATCTGAT |
| | | GTAGTTGTTCTGTTAGCTGCTGAAGAAAAAAAAGAACAGCCG |
| | | ATTGAAACAACAGAAGAAATGGTTGAGCTGACTGAAATAGCT |
| | | TCCCAACCAAAGAAAGAAGAAGACATTGAAATTATTCCAGTC |
| | | CAAGAAGAAGAAGAGGAACTGGAAATAAACTTTGCAGAACCT |
| | | CCCCAGGAGCAGGAATCTTCACCAATAGAAAACGACAGCAT |
| | | CCCTTAA |
| 27 | Amino acid | MTTPRNSMSGTLPVDPMKSPTAMYPVQKIIPKRMPSVVGPTQN |
| | sequence of | FFMRESKTLGAVQIMNGLFHIALGSLLMIHTDVYAPICITMWYPL |
| | canine CD20 | WGGIMFIISGSLLAAADKNPRKSLVKGKMIMNSLSLFAAISGIIFLI |
| | coding region | MDIFNITISHFFKMENLNLIKAPMPYVDIHNCDPANPSEKNSLSIQ |
| | | YCGSIRSVFLGVFAVMVIFTFFQKLVTAGIVENEWKKLCSKPKSD |
| | | VVVLLAAEEKKEQPIETTEEMVELTEIASQPKKEEDIEIIPVQEEE |
| | | EELEINFAEPPQEQESSPIENDSIP |
| 28 | Genomic | TGCAGCCGCAGAAACACAAGGCCCTGGGGCGGCAGAGAGC |
| | sequence of | TATGCCGGGCGGCTGGTCTGCAGACTTCCTGGAGGGCCACC |
| | IGHG1 | CCAAGCCCGGGCACCTGATACCCTGCCCGGACCCTCCCAG |
| | Coding regions | GAGCGAGGCCCTGGTGGTCACACAGCCTCCTCTCTTGCAGC |
| | are indicated in | CTCCACCACGGCCCCATCGGTGTTCCCACTGGCCCCCAGCT |
| | bold and are | GCGGGACCACATCTGGCGCCACCGTGGCCCTGGCCTGCCT |
| | underscored | GGTGTTAGGCTACTTCCCTGAGCCGGTGACCGTGTCCTGGA |
| | | ACTCCGGCGCCCTGACCAGCGGTGTGCACACCTTCCCGGC |
| | | CGTCCTGCAGGCCTCGGGGCTGTACTCTCTCAGCAGCATG |
| | | GTGACAGTGCCCTCCAGCAGGTGGCTCAGTGACACCTTCA |
| | | CCTGCAACGTGGCCCACCCGCCCAGCAACACCAAGGTGGA |
| | | CAAGACCGGTAAGAGGGTGTCCACTGGGAGACAGGCCAGG |
| | | TCAGCCGGTCCACCTGGACCCCGGGGCACAGGAGAGGCAC |
| | | CTCTGTCCCCCAAGCTCGGGGAGGAGGGCCTCTGGGCCTG |
| | | GCTGCCTGGTCCAGTGTGAGCACAGGCTGCACAGCCCCACC |
| | | ATACTTCATCTCCACTGGCAGGCACCAGTCTGGGACGCCCC |
| | | CCTGACCTGCCCTGACCTAGCCCACCGCAAGAGCTGTCCCC |
| | | ACCCCTGGCCCCCACTAACCTCCAGTCTGGTCTCTCTGCAGT |
| | | GCGCAAAACAGACCACCCACCGGGACCCAAACCCTGCGAC |
| | | TGTCCCAAATGCCCACGTGAGTCACCAGGGCACCACCTTGC |
| | | ACACCAAGGCGATGGCCCGCGTCCAGGGACTGCCTGGTGG |
| | | GGGGAATGTTCAAGAGGAGCACCCCCCAAATGCTGACCCCT |
| | | GCATTGTGTCTTCCACACCAGCCCCTGAGATGCTTGGAGGA |
| | | CCGTCCATCTTCATCTTCCCCCCAAAACCCAAGGACACCCT |
| | | CTCGATTTCCCGGACGCCCGAGGTCACATGCTTGGTGGTGG |
| | | ACTTGGGCCCAGATGACTCCGATGTCCAGATCACATGGTTT |
| | | GTGGATAACACCCAGGTGTACACAGCCAAGACGAGTCCGC |
| | | GTGAGGAGCAGTTCAACAGCACCTACCGTGTGGTCAGTGT |
| | | CCTCCCCATCCTACACCAGGACTGGCTCAAGGGGAAGGAG |
| | | TTCAAGTGCAAGGTCAACAGCAAATCCCTCCCCTCCCCCAT |
| | | CGAGAGGACCATCTCCAAGGCCAAAGGTGGGCAACAGGAC |
| | | AGATGGGGCACAGGGAGGTCGAGTGGGGCCTGGCGGACCC |
| | | AGGCCAGCCCTCCACTCTGGGAGTGACCATCTGTGCTGACC |
| | | TCTGACCCCACAGGACAGCCCCACGAGCCCCAGGTGTACGT |
| | | CCTGCCTCCAGCCCAGGAGGAGCTCAGCAGGAACAAAGTC |
| | | AGTGTGACCTGCCTGATCAAAAGCTTCCACCCGCCTGACAT |
| | | TGCCGTCGAGTGGGAGATCACCGGACAGCCGGAGCCAGA |
| | | GAACAACTACCGGACGACCCCGCCCCAGCTGGACAGCGAC |
| | | GGGACCTACTTCGTGTACAGCAAGCTCTCGGTGGACAGGT |
| | | CCCACTGGCAGAGGGGAAACACCTACACCTGCTCGGTGTC |
| | | ACACGAAGCTCTGCACAGCCACCACACACAGAAATCCCTC |
| | | ACCCAGTCTCCGGGTAAATGAGCAGCGCGCCCAGCCCCCC |
| | | AGGAGGCCCCCGCGGGCTCTGAGCGCCCACCCCTGTGTAC |
| | | ATCCCCCACCCCGGGCAGGTACCCTGCGTGAAATAAAGCAC |
| | | CCAGCACTGCCCTGGGACCTAGGACACTGTCGTGCGTCTTT |
| | | CCAAGGCAGAGCTCAGGACACCCGGGCCTCTGGGAGGTGC |
| | | GGGCAGGCCAGGCTCCGAGGCTCAGGGGTCACCAT |
| 29 | Genomic | TGCAGCCGCAGAAACACGAGGCCCTGGGGCGGCAGAGAGC |
| | sequence of | TACGCCGGGCGGCTGGTCTGCAGACTTCCTGGAGGGCCAC |
| | IGHG2 | CCCAAGCCCGGGCACCTGATAACCTGCCCGGACCCTCCCAG |
| | Coding regions | GAGCGAGGCCCTGGTGGTCACACAGCCTCCTCTCTTGCAGC |
| | are indicated by | CTCCCCCACGGCCCCATCGGTGTTCCCACTGGCCCCCAGCT |
| | an underscore. | GCGGGACCACATCTGGCGCCACCGTGGCCCTGGCCTGCCT |
| | | GGTGTTAGGCTACTTCCCTGAGCCGGTGACCGTGTCCTGGA |
| | | ACTCCGGCGCCCTGACCAGCGGTGTGCACACCTTCCCATC |
| | | CGTCCTGCAGGCCTCGGGGCTCTACTCTCTCAGCAGCATGG |
| | | TGACAGTGCCCTCCAGCAGGTGGCTCAGTGACACCTTCACC |
| | | TGCAACGTGGCCCACCGGCCCAGCAGCACCAAAGTGGACA |
| | | AGACCGGTAAGAGGGTGTCCACTGGGAGACAGGCCAGGTC |
| | | AGCCGGTCCACCTGGACCCCGGGGCACAGGAGAGGCACCT |
| | | CTGTCCCCCAAGCTCGGGGAGGAGGGCCTCTGGGCCTGGC |
| | | TGCCTGGTCCAGTGTGAGCACAGGCTGCACAGCCCCACCAT |
| | | ACTTCATCTCCACTGGCAGGCACCAGTCTGGGACGCCCCCC |
| | | TGACCTGCCCTGACCTAGCCCACCCCAAGAGCTGTCCCCAC |
| | | CCCTGGCCCCCACTAACCTCCAGTCTGGTCTCTCTGCAGTA |
| | | CCCAAAACAGCGTCTACAATAGAGTCCAAAACCGGGGAAG |
| | | GTCCCAAATGCCCAGGTGAGTCACCAGGGCACCACCTTGCA |
| | | CACCAAGGCAATGGCCCGCATCCAGGGACTGCCTGGTGGG |
| | | GGGAATGTTCAAGAGGATCCCCCCCAAATGCTGACCCCTGC |
| | | ATTGTGTCTCCCACACCAGTTCCTGAGATTCCTGGAGCACC |
| | | GTCCGTCTTCATCTTCCCCCCAAAACCCAAGGACACCCTCT |
| | | CGATTTCCCGGACGCCCGAGGTCACGTGCTTGGTGGTGGA |
| | | CTTGGGCCCAGATGACTCCAATGTCCAGATCACATGGTTTG |
| | | TGGATAACACCGAGATGCACACAGCCAAGACGAGGCCGCG |
| | | TGAGGAGCAGTTCAACAGCACCTACCGTGTGGTCAGTGTCC |
| | | TCCCCATCCTACACCAGGACTGGCTCAAGGGGAAGGAGTT |
| | | CAAGTGCAAGGTCAACAGCAAATCCCTCCCCTCTGCCATGG |
| | | AGAGGACCATCTCCAAGGCCAAAGGTGGGCAACAGGACAG |
| | | ATGGGGCACAGGGAGGTCGAGTGGGGCCTGGTGGACCCAG |
| | | GCCAGCCCTCCACTCTGGGAGTGACCATCTGTGCTGACCTC |
| | | TGACCCCACAGGACAGCCCCACGAGCCCCAGGTGTACGTC |
| | | CTGCCTCCAACCCAGGAGGAGCTCAGCGAGAACAAAGTCA |
| | | GTGTGACCTGCCTGATCAAAGGCTTCCACCCGCCTGACATT |
| | | GCCGTCGAGTGGGAGATCACCGGACAGCCGGAGCCAGAG |
| | | AACAACTACCAGACGACCCCGCCCCAGCTGGACAGCGACG |
| | | GGACCTACTTCCTGTACAGCAGGCTCTCGGTGGACAGGTCC |
| | | CACTGGCAGAGGGGAAACACCTACACCTGCTCGGTGTCAC |
| | | ACGAAGCTCTGCACAGCCACCACACACAGAAATCCCTCAC |
| | | CCAATCTCCGGGTAAATGAGCAGCGCGCCCAGCCCCCCAG |
| | | GAGGCCCCCGCGGGCTCTGAGCGCCCACCCCTGTGTACAT |
| | | CCCCCCCCCCCGGGCAGGCACCCTGCATGAAATAAAGCACC |
| | | CAGCACTGCCCTGGGACCTGTGACACTGTCGTGCGTCTTTC |
| | | CAAGGCAGAGCTCAGGACACCCGGGCCTCTGGGAGGTGCG |
| | | GGCAGGCCAGGCTCTGAGGCTCAGGGGTCACCAT |
| 30 | Genomic | CAGCCGCAGAAACATGAGGTCCTGGGGCGGCAGAGAGCTA |
| | sequence of | CGCCGGGCGGCTGGTCTGCAGACTTCCTGGAGGGCCACCC |
| | IGHG3 | CAAGCCCGGGCACCTGATACCCTGCCCGGACCCTCCCAGGA |
| | Coding regions | GCGAGGCCCTGGTGGTCACACAGCCTCCTCTCTTGCAGCCT |
| | are indicated by | CCCCCAAGGCCCCATCGGTGTTCCCACGGGCCCCCAGCTG |
| | an underscore. | TGGGACCATATCTGGCGCCACCGTGGCCCTGATCTGCCTG |
| | | GTGTTAGACTACTTCCCTGAGCCGGTGACCGTGTCCTGGAA |
| | | GCCCGGCGCCCTGACCAACGGTGTGCACACCTTCCCGGCC |
| | | GTCCTGCAGCCCTCGGGGCTCTACTCTCTCAGCAGCATGGT |
| | | GACAGTGCCCACCAGCAGCTGGCTCAGAGACACCTTCACC |
| | | TGCAACGTGGCCCACCCGCCCAGCAACACCAAGGTGGAAAA |
| | | GATCGGTAAGAGGGTGTCCACTGGGAGACAGGCCAGGTCA |
| | | GCCGGTCCACCTGGACCCCGGGGCACAGGAGAGGCACCTC |
| | | TGTCCCCCAAGCTCGGGGAGGAGGGCCTCTGGGCCTGGCT |
| | | GCCTGGTCCAGTGTGAGCACAGGCTGCACAGCCCCACCATA |
| | | CTTCATCTCCACTGGCAGGCACCAGTCTGGGACGCCCCCCT |
| | | GACCTGCCCTGACCTAGCCCACCCCAAGAGCTGTCCCCACC |
| | | CCTGGCCCCCACTAACCTCCAGTCTGGTCTCTCTGCAGTGCT |
| | | CCCACCCTGCAAGTGTCCCAAATGCCCAGGTGAGTCAGCA |
| | | GGGCACCGCCCTGCACACCAAGGCCATGGTCCGTATCCAGG |
| | | GACTGCCTGGTGGGGGGAATGTTCAAGAGGAGCACCCCCCA |
| | | AATGCTGACCCCCGCATTGTGTCTCCCACACCAGTACCTGA |
| | | GATTCCCGGGGGACCGTCCGTCTTCATCTTCCCCCCAAAAC |
| | | CCAAGGACACCCTCTCGATTTCCCGGACGCCCGAGGTCAC |
| | | GTGCTTGGTGGTGGACTTGGGCCCAGATGACTCCAATGTCC |
| | | AGATCACATGGTTTGTGGATAACACCGAGATGCACACAGCC |
| | | AAGACGAGACCGCGTGAGGAGCAGTTCAACAGCACCTACC |
| | | GTGTGGTCAGTGTCCTCCCCATCGTGCACCAGGACTGGCTC |
| | | ACGGGGAAGGAGTTCAAGTGCAAGGTCAACAGCAAAGCCC |
| | | TCCCCTCTGCCATCGAGAGGACCATCTCCAAGGCCAAAGG |
| | | TGGGCAACAGGACAGATGGGGCACAGGGAGGTCGAGTGGG |
| | | GCCTGGCGGACCCAGGCCAGCCCTCCACTCTGGGAGTGAC |
| | | CATCTGTGCTGACCTCTGACCCCACAGGACAGCCCCACGAG |
| | | CCCCAGGTGTACGTCCTGCCTCCAGCCCAGGAGGAGCTCA |
| | | GCGAGAACAAAGTCTGTGTGACCTGCCTGATCAAAGGCTTC |
| | | TACCCGCCTGACATTGCCGTCGAGTGGGAGATCACCGGAC |
| | | AGCCGGAGCCAGAGAACAACTACCGGACGACCCCGCCCC |
| | | AGCTGGACAGCGACGGGACCTACTTCGTGTACAGCAGGCT |
| | | CTCGATGGACAGGTCCCGCTGGCAGAGTGGAAACACCTAC |
| | | ACCTGCTCGGTGTCACACGAAGCTCTGCACAGCCACCACA |
| | | CACAGAAATCCCTCACCCAGTCTCCGGGTAAATGAGCAGC |
| | | GCGCCCAGCCCCCCAGCAGGCCCCCGCGGGCTCTGAGCGC |
| | | CCACCCCTGTGTACATCCCCCACCCCGGGCAGGTACCCTGC |
| | | GTGAAATAAAGCACCCAGCACTGCCCTGGGACCTGTGACAC |
| | | TGTCGTGTGTCTTTCCAAGGCAGAGCTCAGGACACCCGGGC |
| | | CTCTGGGAGGTGCGGGCAGGCCAGGCTCCAAGGCTCAGGG |
| | | GTCACCAT |
| 31 | Feline IgG 1 | STTAPSVFPLAPSCGTTSGATVALACLVLGYFPEPVTVSWNSG |
| | | ALTSGVHTFPAVLQASGLYSLSSMVTVPSSRWLSDTFTCNVAH |
| | | PPSNTKVDKTVRKTDHPPGPKPCDCPKCPPPEMLGGPSIFIFPP |
| | | KPKDTLSISRTPEVTCLVVDLGPDDSDVQITWFVDNTQVYTAKT |
| | | SPREEQFNSTYRVVSVLPILHQDWLKGKEFKCKVNSKSLPSPIE |
| | | RTISKAKGQPHEPQVYVLPPAQEELSRNKVSVTCLIKSFHPPDIA |
| | | VEWEITGQPEPENNYRTTPPQLDSDGTYFVYSKLSVDRSHWQ |
| | | RGNTYTCSVSHEALHSHHTQKSLTQSPGK |
| 32 | Feline IgG 2 | SPTAPSVFPLAPSCGTTSGATVALACLVLGYFPEPVTVSWNSG |
| | | ALTSGVHTFPSVLQASGLYSLSSMVTVPSSRWLSDTFTCNVAH |
| | | RPSSTKVDKTVPKTASTIESKTGEGPKCPVPEIPGAPSVFIFPPK |
| | | PKDTLSISRTPEVTCLVVDLGPDDSNVQITWFVDNTEMHTAKTR |
| | | PREEQFNSTYRVVSVLPILHQDWLKGKEFKCKVNSKSLPSAME |
| | | RTISKAKGQPHEPQVYVLPPTQEELSENKVSVTCLIKGFHPPDIA |
| | | VEWEITGQPEPENNYQTTPPQLDSDGTYFLYSRLSVDRSHWQ |
| | | RGNTYTCSVSHEALHSHHTQKSLTQSPGK |
| 33 | Feline IgG 3 | SPKAPSVFPRAPSCGTISGATVALICLVLDYFPEPVTVSWKPGA |
| | | LTNGVHTFPAVLQPSGLYSLSSMVTVPTSSWLRDTFTCNVAHP |
| | | PSNTKVEKIVLPPCKCPKCPVPEIPGGPSVFIFPPKPKDTLSISRT |
| | | PEVTCLVVDLGPDDSNVQITWFVDNTEMHTAKTRPREEQFNST |
| | | YRVVSVLPIVHQDWLTGKEFKCKVNSKALPSAIERTISKAKGQP |
| | | HEPQVYVLPPAQEELSENKVCVTCLIKGFYPPDIAVEWEITGQP |
| | | EPENNYRTTPPQLDSDGTYFVYSRLSMDRSRWQSGNTYTCSV |
| | | SHEALHSHHTQKSLTQSPGK |
| 34 | Ofa-VH-fIgG1- | MELGLSWIFLLAILKGVQCEVQLVESGGGLVQPGRSLRLSCAAS |
| | Def1: | GFTFNDYAMHWVRQAPGKGLEWVSTISWNSGSIGYADSVKGR |
| | | FTISRDNAKKSLYLQMNSLRAEDTALYYCAKDIQYGNYYYGMDV |
| | | WGQGTTVTVSSASTTAPSVFPLAPSCGTTSGATVALACLVLGY |
| | | FPEPVTVSWNSGALTSGVHTFPAVLQASGLYSLSSMVTVPSSR |
| | | WLSDTFTCNVAHPPSNTKVDKTVRKTDHPPGPKPCDCPKCPP |
| | | PEAAGGPSIFIFPPKPKDTLSISRTPEVTCLVVDLGPDDSDVQIT |
| | | WFVDNTQVYTAKTSPREEQFNSTYRVVSVLPILHQDWLKGKEF |
| | | KCKVNSKSLPSSIERTISKAKGQPHEPQVYVLPPAQEELSRNKV |
| | | SVTCLIKSFHPPDIAVEWEITGQPEPENNYRTTPPQLDSDGTYF |
| | | VYSKLSVDRSHWQRGNTYTCSVSHEALHSHHTQKSLTQSPGK |
| 35 | Ofa-VH-fIgG1- | MELGLSWIFLLAILKGVQCEVQLVESGGGLVQPGRSLRLSCAAS |
| | Def2: | GFTFNDYAMHWVRQAPGKGLEWVSTISWNSGSIGYADSVKGR |
| | | FTISRDNAKKSLYLQMNSLRAEDTALYYCAKDIQYGNYYYGMDV |
| | | WGQGTTVTVSSASTTAPSVFPLAPSCGTTSGATVALACLVLGY |
| | | FPEPVTVSWNSGALTSGVHTFPAVLQASGLYSLSSMVTVPSSR |
| | | WLSDTFTCNVAHPPSNTKVDKTVRKTDHPPGPKPCDCPKCPP |
| | | PEAAGGPSIFIFPPKPKDTLSISRTPEVTCLVVDLGPDDSDVQIT |
| | | WFVDNTQVYTAKTSPREEQFNSTYRVVSVLPILHQDWLKGKEF |
| | | KCKVNSKSLGSPIERTISKAKGQPHEPQVYVLPPAQEELSRNKV |
| | | SVTCLIKSFHPPDIAVEWEITGQPEPENNYRTTPPQLDSDGTYF |
| | | VYSKLSVDRSHWQRGNTYTCSVSHEALHSHHTQKSLTQSPGK |
| 36 | Ofa-VH-fIgG1- | MELGLSWIFLLAILKGVQCEVQLVESGGGLVQPGRSLRLSCAAS |
| | Def3: | GFTFNDYAMHWVRQAPGKGLEWVSTISWNSGSIGYADSVKGR |
| | | FTISRDNAKKSLYLQMNSLRAEDTALYYCAKDIQYGNYYYGMDV |
| | | WGQGTTVTVSSASTTAPSVFPLAPSCGTTSGATVALACLVLGY |
| | | FPEPVTVSWNSGALTSGVHTFPAVLQASGLYSLSSMVTVPSSR |
| | | WLSDTFTCNVAHPPSNTKVDKTVRKTDHPPGPKPCDCPKCPP |
| | | PEIPGAPSIFIFPPKPKDTLSISRTPEVTCLVVDLGPDDSDVQITW |
| | | FVDNTQVYTAKTSPREEQFNSTYRVVSVLPILHQDWLKGKEFK |
| | | CKVNSKSLPSPIERTISKAKGQPHEPQVYVLPPAQEELSRNKVS |
| | | VTCLIKSFHPPDIAVEWEITGQPEPENNYRTTPPQLDSDGTYFV |
| | | YSKLSVDRSHWQRGNTYTCSVSHEALHSHHTQKSLTQSPGK |
| 37 | Ofa-VH-fIgG1- | MELGLSWIFLLAILKGVQCEVQLVESGGGLVQPGRSLRLSCAAS |
| | Def4: | GFTFNDYAMHWVRQAPGKGLEWVSTISWNSGSIGYADSVKGR |
| | | FTISRDNAKKSLYLQMNSLRAEDTALYYCAKDIQYGNYYYGMDV |
| | | WGQGTTVTVSSASTTAPSVFPLAPSCGTTSGATVALACLVLGY |
| | | FPEPVTVSWNSGALTSGVHTFPAVLQASGLYSLSSMVTVPSSR |
| | | WLSDTFTCNVAHPPSNTKVDKTVRKTDHPPGPKPCDCPKCPP |
| | | PEIPGGPSIFIFPPKPKDTLSISRTPEVTCLVVDLGPDDSDVQITW |
| | | FVDNTQVYTAKTSPREEQFNSTYRVVSVLPILHQDWLKGKEFK |
| | | CKVNSKSLPSAIERTISKAKGQPHEPQVYVLPPAQEELSRNKVS |
| | | VTCLIKSFHPPDIAVEWEITGQPEPENNYRTTPPQLDSDGTYFV |
| | | YSKLSVDRSHWQRGNTYTCSVSHEALHSHHTQKSLTQSPGK |
| 38 | Modified feline | ASTTAPSVFPLAPSCGTTSGATVALACLVLGYFPEPVTVSWNS |
| | IgG1 constant | GALTSGVHTFPAVLQASGLYSLSSMVTVPSSRWLSDTFTCNVA |
| | region Def 1 | HPPSNTKVDKTVRKTDHPPGPKPCDCPKCPPPEAAGGPSIFIFP |
| | | PKPKDTLSISRTPEVTCLVVDLGPDDSDVQITWFVDNTQVYTAK |
| | | TSPREEQFNSTYRVVSVLPILHQDWLKGKEFKCKVNSKSLPSSI |
| | | ERTISKAKGQPHEPQVYVLPPAQEELSRNKVSVTCLIKSFHPPDI |
| | | AVEWEITGQPEPENNYRTTPPQLDSDGTYFVYSKLSVDRSHW |
| | | QRGNTYTCSVSHEALHSHHTQKSLTQSPGK |
| 39 | Modified feline | ASTTAPSVFPLAPSCGTTSGATVALACLVLGYFPEPVTVSWNS |
| | IgG1 constant | GALTSGVHTFPAVLQASGLYSLSSMVTVPSSRWLSDTFTCNVA |
| | region Def 2 | HPPSNTKVDKTVRKTDHPPGPKPCDCPKCPPPEAAGGPSIFIFP |
| | | PKPKDTLSISRTPEVTCLVVDLGPDDSDVQITWFVDNTQVYTAK |
| | | TSPREEQFNSTYRVVSVLPILHQDWLKGKEFKCKVNSKSLGSPI |
| | | ERTISKAKGQPHEPQVYVLPPAQEELSRNKVSVTCLIKSFHPPDI |
| | | AVEWEITGQPEPENNYRTTPPQLDSDGTYFVYSKLSVDRSHW |
| | | QRGNTYTCSVSHEALHSHHTQKSLTQSPGK |
| 40 | Modified feline | ASTTAPSVFPLAPSCGTTSGATVALACLVLGYFPEPVTVSWNS |
| | IgG1 constant | GALTSGVHTFPAVLQASGLYSLSSMVTVPSSRWLSDTFTCNVA |
| | region Def 3 | HPPSNTKVDKTVRKTDHPPGPKPCDCPKCPPPEIPGAPSIFIFP |
| | | PKPKDTLSISRTPEVTCLVVDLGPDDSDVQITWFVDNTQVYTAK |
| | | TSPREEQFNSTYRVVSVLPILHQDWLKGKEFKCKVNSKSLPSPI |
| | | ERTISKAKGQPHEPQVYVLPPAQEELSRNKVSVTCLIKSFHPPDI |
| | | AVEWEITGQPEPENNYRTTPPQLDSDGTYFVYSKLSVDRSHW |
| | | QRGNTYTCSVSHEALHSHHTQKSLTQSPGK |
| 41 | Modified feline | ASTTAPSVFPLAPSCGTTSGATVALACLVLGYFPEPVTVSWNS |
| | IgG1 constant | GALTSGVHTFPAVLQASGLYSLSSMVTVPSSRWLSDTFTCNVA |
| | region Def 4 | HPPSNTKVDKTVRKTDHPPGPKPCDCPKCPPPEIPGGPSIFIFP |
| | | PKPKDTLSISRTPEVTCLVVDLGPDDSDVQITWFVDNTQVYTAK |
| | | TSPREEQFNSTYRVVSVLPILHQDWLKGKEFKCKVNSKSLPSAI |
| | | ERTISKAKGQPHEPQVYVLPPAQEELSRNKVSVTCLIKSFHPPDI |
| | | AVEWEITGQPEPENNYRTTPPQLDSDGTYFVYSKLSVDRSHW |
| | | QRGNTYTCSVSHEALHSHHTQKSLTQSPGK |

1-49. (canceled)

50. A canine or caninised IgG-B polypeptide or Fc region thereof comprising a modified Fc region wherein said Fc region comprises
a) an amino acid substitution selected from one or more of the following substitutions to SEQ ID NO: 11: G at position 119; S or A at position 120; and A at position 121; and optionally
b) N at position 156; and further optionally
c) an amino acid substitution selected from one of the following substitutions to SEQ ID NO: 11:
c1) H at position 211 in combination with I at position 212 and G at position 213;
c2) G at position 215;
c3) S at position 217; and
c4) a combination of c1 to c3;
wherein the Fc region is not an Fc region with only two substitutions namely A at positions 120 and 121 of SEQ ID NO: 11, and wherein said Fc region is the Fc region of a canine antibody that binds canine CD20.

51. A canine or caninised IgG-B polypeptide or Fc region thereof of claim 50, wherein said Fc region comprises an amino acid substitution as set out in a) below and optionally an amino acid substitution as set out in b) below and optionally c) below wherein:
a) the amino acid substitution is selected from one or more of the following substitutions to SEQ ID NO: 11: G at position 119; S or A at position 120; and A at position 121;
b) the amino acid substitution is selected from one or more of the following substitutions to SEQ ID NO: 11: G at position 153; R at position 154; and N at position 156; and optionally
c) the amino acid substitution is selected from one or more substitutions to SEQ ID NO:11: H at position 211; I at position 212; G at position 213; G at position 215; and S at position 217.

52. A canine or caninised IgG-B polypeptide or Fc region thereof of claim 50, wherein said Fc region comprises the following amino acid substitutions to SEQ ID NO: 11:
a) S at position 120;
b) G at position 153 and R at position 154; and
c) H at position 211, I at position 212; and G at position 213; and optionally one of the following substitutions: S at position 217 and G at position 215.

53. A canine or caninised IgG-B polypeptide or Fc region thereof of claim 50, wherein said Fc region comprises the following amino acid substitutions to SEQ ID NO: 11:
a) A at positions 120 and 121; and optionally
b) one of the following substitutions: S at position 217 and G at position 215.

54. A canine or caninised IgG-B polypeptide or Fc region thereof of claim 50, wherein said Fc region comprises the following substitutions to SEQ ID NO: 11:
a) G at position 119; and
b) N at position 156.

55. A canine or caninised IgG-B polypeptide or Fc region thereof of claim 50, wherein said Fc region comprises the following substitutions to SEQ ID NO: 11:
a) A at positions 120 and 121;
b) N at position 156; and
c) S at position 217.

56. A canine or caninised IgG-B polypeptide or Fc region thereof of claim 50, wherein said Fc region comprises the following substitutions to SEQ ID NO: 11:
a) S at position 120; and
b) H at position 211, I at position 212 and G at position 213.

57. A canine or caninised IgG-B polypeptide or Fc region thereof of claim 50, wherein said polypeptide comprises SEQ ID NOs: 12, 13, 14, 15, 16, 17, 18, 19 or 20 or a sequence with at least 90% or 95% sequence identity thereto.

58. A canine or caninised IgG-B polypeptide or Fc region thereof of claim 50, wherein the polypeptide has reduced Fc-mediated effector function when compared to the same polypeptide comprising a wild-type IgG-B Fc domain, wherein the effector function is antibody-dependent cell-mediated cytotoxicity (ADCC), complement-dependent cytotoxicity (CDC), or antibody-dependent cell-mediated phagocytosis (ADCP).

59. A canine or caninised IgG-B polypeptide or Fc region thereof of claim 50, wherein the polypeptide has lower affinity for an Fc gamma receptor (FcγR) when compared to the same polypeptide comprising a wild-type IgG1 Fc domain.

60. A canine or caninised IgG-B polypeptide or Fc region thereof of claim 50, wherein the polypeptide or Fc region retains FcRn binding.

61. A pharmaceutical composition comprising a polypeptide or Fc region thereof of claim 50.

62. An in vitro, ex vivo or in vivo method for repressing effector function comprising contacting a cell or tissue with a polypeptide or Fc region thereof of claim 50, wherein the effector function is antibody-dependent cell-mediated cytotoxicity (ADCC), or complement-dependent cytotoxicity (CDC).

63. A nucleic acid encoding a polypeptide or Fc region thereof of claim 50.

64. A vector comprising a nucleic acid or Fc region thereof of claim 63.

65. A host cell comprising a nucleic acid of claim 63.

66. A kit comprising polypeptide or Fc region thereof of claim 50.

67. A feline or felinised IgG-1 polypeptide or Fc region thereof comprising a modified Fc region wherein said Fc region comprises the following substitutions to SEQ ID NO: 31:
a) A at positions 120 and 121; or I at position 120 and P at position 121; and optionally
b) one or more of the following substitutions: S or A at position 217, G at position 215, and A at position 123.

68. A feline or felinised IgG-1 polypeptide or Fc region thereof of claim 67 comprising a modified Fc region wherein said Fc region comprises
a) the following substitutions to SEQ ID NO: 31: A at positions 120 and 121; and further comprising either S at position 217 or G at position 215; or
b) the following substitutions to SEQ ID NO: 31: I at position 120; P at position 121; and further comprising either A at position 217 or A at position 123.

69. A pharmaceutical composition comprising a polypeptide or Fc region thereof of claim 67.

70. A nucleic acid encoding a polypeptide or Fc region thereof of claim 67.

71. A vector comprising a nucleic acid of claim 70.

72. A host cell comprising a nucleic acid of claim 70.

73. A kit comprising the polypeptide or Fc region thereof of claim 67.

74. A method of treating an inflammatory or autoimmune disease in a subject comprising administering an effective amount of the feline or felinised polypeptide or Fc region thereof of claim 67.

75. An isolated IgG-3 feline polypeptide comprising SEQ ID NO: 33 or an Fc region thereof.
